(19) United States
(12) Patent Application Publication
(10) Pub. No.: US 2011/0065687 A1
(43) Pub. Date: Mar. 17, 2011

(54) HYDRAZIDE COMPOUNDS AND USES THEREOF

(76) Inventors: Michael Schwaebe, San Diego, CA (US); Johnny Yasuo Nagasawa, San Diego, CA (US); Mustapha Haddach, San Diego, CA (US)

(21) Appl. No.: 12/596,240

(22) PCT Filed: Apr. 17, 2008
(86) PCT No.: PCT/US08/60677

§ 371 Date: Dec. 1, 2010

Related U.S. Application Data
(60) Provisional application No. 60/912,411, filed on Apr. 17, 2007.

(51) Int. Cl. A61K 31/5377; C07D 513/14; A61K 31/497; A61K 31/496; A61K 31/551; C07D 498/04; C07D 471/04; C07D 513/04; C07D 498/14; A61K 31/5383; A61K 31/4985; A61K 31/542; A61P 35/00; A61P 31/12; A61P 31/04; A61P 31/10

(52) U.S. Cl. 514/218; 544/121; 544/357; 544/80; 544/125; 544/361; 540/575; 514/233.2; 514/252.11; 514/232.5; 514/253.02; 544/99; 544/342; 544/14; 544/74; 514/229.5; 514/250; 514/224.5

(57) ABSTRACT

This application relates to certain novel polycyclic compounds that interact with quadruplex-forming regions of polynucleotides and thereby inhibit translation of genetic information into polypeptides. These compounds can thus provide anticancer and antibacterial and antiviral effects. The invention includes novel compounds and pharmaceutical compositions, and methods of using them to treat cancer and other conditions.

### FIELD OF THE INVENTION

The invention relates to certain hydrazides and related compounds described herein, and methods of using such compounds as pharmaceutical agents.

### BACKGROUND

Proteins in cells are synthesized in a process referred to as “translation.” Proteins are translated from messenger ribonucleic acids (mRNAs), the latter having been transcribed from deoxyribonucleic acid (DNA) nucleotide sequences. Each protein is synthesized as a chain of amino acids, and in the translation process ribosomes bind to and travel along the mRNA and sequentially add each amino acid in the chain. A ribosome bound to an mRNA selects a tRNA-loaded amino acid according to nucleotide triplets (i.e., codons) sequentially arranged along the mRNA.

A human ribosome is an 80S particle that comprises a 60S large subunit and a 40S small subunit. The “S” designation in “80S,” “60S” and “40S” refers to a “Svedberg unit,” a sedimentation measure of particle size. Each ribosome subunit is an assembly of proteins and functional RNA, which serves as a docking region for tRNA-loaded amino acids. The functional RNA is referred to as “ribosomal RNA (rRNA)” and it is synthesized by polymerase I and III enzymes that utilize a region of genomic DNA, referred to as “ribosomal DNA (rDNA),” as a template. The rDNA sequence is repeated approximately 400 times in the human genome. Ribosomal RNA biogenesis begins with the synthesis of a 47S precursor rRNA, which is iteratively cleaved into smaller, mature 18S, 5.8S and 28S rRNA by the coordinated action of a variety of endonucleases, exonucleases, RNA helicases and other protein factors. The 18S rRNA is assembled into the 40S ribosomal subunit and the 28S and 5.8S rRNA are assembled into the 60S ribosomal subunit. Human ribosome biogenesis occurs mainly in the nucleolus, a specialized compartment in the cell nucleus.

### SUMMARY OF THE INVENTION

Compounds described herein act as tumor suppression agents for the treatment of cancers with reduced side effects. Compounds provided herein can interact with regions of DNA that regulate transcription of rRNA and can bind to nucleotide sequences capable of forming quadruplex structures. The compounds may also exhibit antibacterial or antiviral activity, and may be used for treating bacterial and viral infections. Various embodiments of the present invention are described below.

In one aspect, the compounds have the general formula:

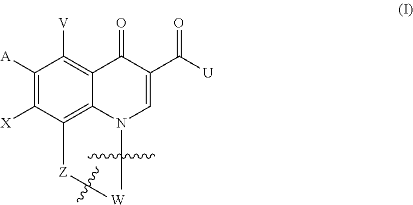

and pharmaceutically acceptable salts, esters and prodrugs thereof;

wherein V is H, halo, or NR¹R²;

A is H, fluoro, or NR¹₂;

Z is O, S, NR¹ or CH₂;

U is —NR¹NR¹R² or —NR¹OR²;

X is OR², NR¹R², —NR¹—NR¹R², —NR¹—OR², halo, azido, SR² or an optionally substituted seven-membered carbocyclic or azacyclic ring;

n is 1-3;

wherein in NR¹R², R¹ and R² may form a double bond or a ring, each of which is optionally substituted;

each R¹ is independently H or a C₁₋₆ alkyl;

each R² is independently H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring;

or R² is an optionally substituted heterocyclic ring, aryl or heteroaryl;

W is selected from the group consisting of

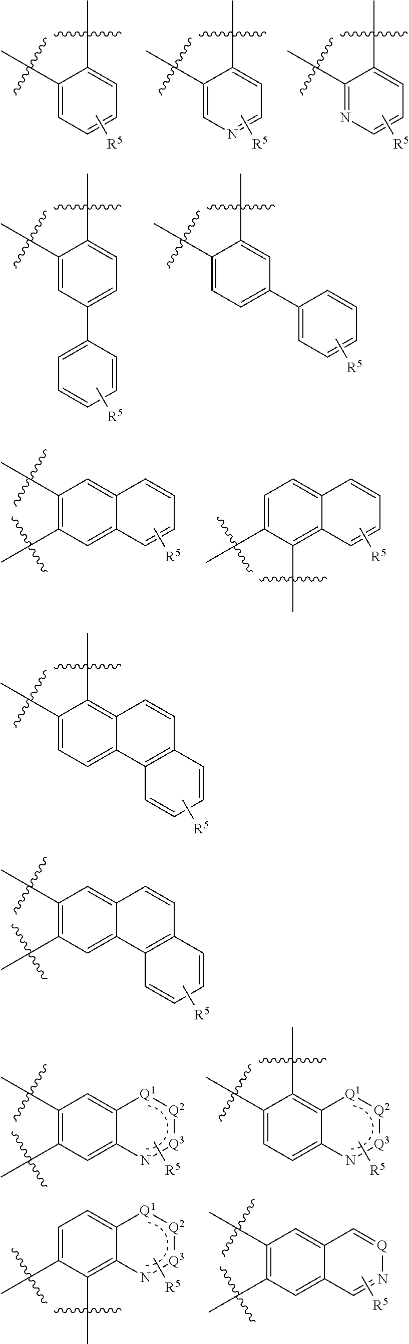

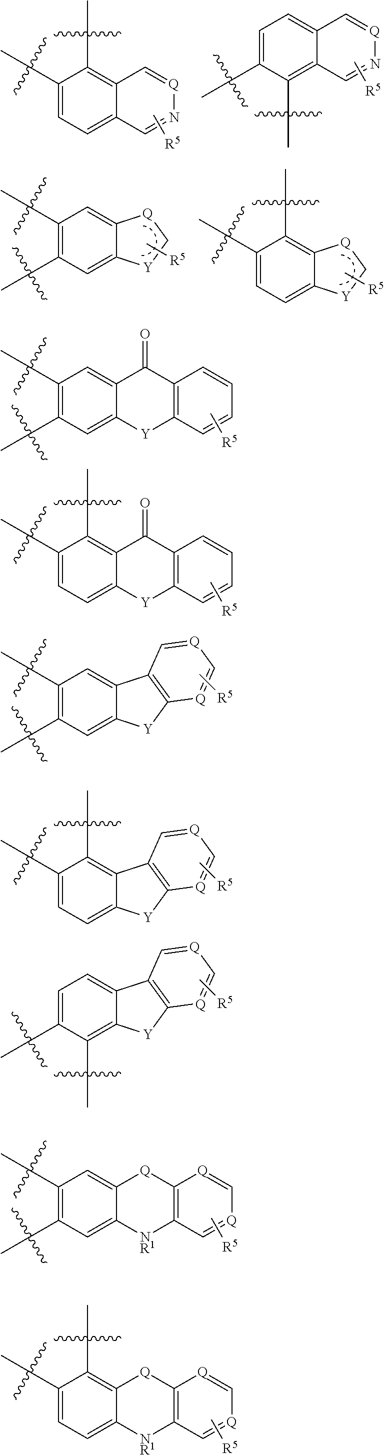

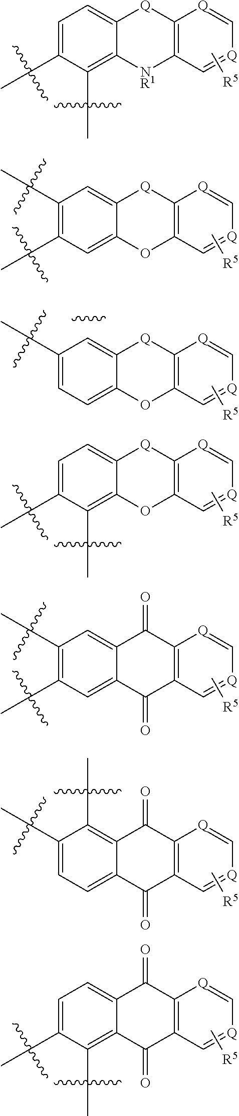

wherein Q, Q¹, Q², and Q³ are independently CH or N;
Y is independently O, CH, ═O or NR¹;
and R⁵ is a substituent at any position on the fused ring; and is H, OR², C₁₋₆ alkyl, C₂₋₆ alkenyl, each optionally substituted by halo, ═O or one or more heteroatoms; or R⁵ is an inorganic substituent; or two adjacent R⁵ groups are linked to obtain a 5-6 membered substituted or unsubstituted carbocyclic or heterocyclic ring, optionally fused to an additional substituted or unsubstituted carbocyclic or heterocyclic ring.

The invention includes the individual compounds of this formula, as well as mixtures thereof. Where a chiral center (or more than one) is present, it includes each individual isomer as well as mixtures such as racemic mixtures and the like. It also includes salts of these compounds having pharmaceutically acceptable counterions, and in particular it includes the salts formed by addition of H-PA to the compounds of the above formula, where PA represents a pharmaceutically acceptable anion.

In the above formula I, A and X may independently be halo. In one example, A and X may independently be fluoro.

In the above formula, V may be H. Alternatively, V may be NH₂ or a compound having the formula NR¹—(CR¹₂)ₙ—NR³R⁴;

wherein R¹ and R³ are independently H or C₁₋₆ alkyl;

n is 1-6; and

R⁴ is H, C₁₋₆ alkyl optionally substituted with a carbocyclic or heterocyclic ring, or aryl; and wherein in NR³R⁴, R³ and R⁴ may form an optionally substituted ring.

In the above formula I, U can be NR¹NR¹R² or NR¹—OR². In one example, R¹ is H and R² is a C₁₋₁₀ alkyl optionally containing one or more heteroatoms, and optionally substituted with a C₃₋₆ cycloalkyl, aryl or a 5-14 membered heterocyclic ring containing one or more N, O or S; the latter rings can be either substituted or unsubstituted. In another example, R¹ is H and R² is an aryl or a 5-14 membered heterocyclic ring containing one or more N, O or S, each optionally substituted with an amino or another heterocyclic ring. In yet another example, R¹ and R² in NR¹R² form an optionally substituted 5-14 membered ring containing one or more N, O or S. In particular examples, NR¹R² is morpholine, thiomorpholine, piperazine, piperidine or diazepine.

In some embodiments, U is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. In other embodiments, NR¹R² of U is an acyclic group, wherein R¹ is H or an optionally substituted C₁-C₆ alkyl such as methyl or ethyl or methoxymethyl or trifluoroethyl, and R² represents a ring such as pyridine, phenyl, pyrazine, piperidine, pyrrolidine, pyrrole, morpholine, piperazine, imidazole, imidazolidine, triazine, pyrazole, and the like.

In the above formula I, U can have the formula

NR¹NR¹—(CR¹₂)ₙ—NR³R⁴ or NR¹O—(CR¹₂)ₙ—NR³R⁴ (2)

wherein R¹ and R³ are independently H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR³R⁴, R³ and R⁴ may form an optionally substituted ring.

In the above formula I, X can be NR¹R². In one example, R¹ is H and R² is a C₁₋₁₀ alkyl optionally containing one or more heteroatoms, and optionally substituted with a C₃₋₆ cycloalkyl, aryl or a 5-14 membered heterocyclic ring containing one or more N, O or S, where each of these rings is optionally substituted. In another example, R¹ is H and R² is an aryl or a 5-14 membered heterocyclic ring containing one or more N, O or S, each optionally substituted with an amino or another heterocyclic ring. In yet another example, R¹ and R² in NR¹R² form an optionally substituted 5-14 membered ring containing one or more N, O or S. In particular examples, NR¹R² is morpholine, thiomorpholine, piperazine, piperidine or diazepine.

In the above formula 1, X can have the formula

NR¹—(CR¹₂)ₙ—NR³R⁴ (2)

wherein R¹ and R³ are independently H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR³R⁴, R³ and R⁴ may form an optionally substituted ring.

In the above formula 2, n may be 2-3. In one example, NR³R⁴ is an acyclic amine, or guanidinyl or a tautomer thereof; or R³ and R⁴ optionally form a substituted ring containing one or more N, O or S. In particular examples, NR³R⁴ is morpholine, thiomorpholine, imidazole, pyrrolidine, piperazine, pyridine or piperidine.

In the above formula I, X may be NR¹R²; and U has the formula

NR¹NR¹—(CR¹₂)ₙ—NR³R⁴ (2)

wherein R¹ and R² are as defined in claim 1;

R³ is H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR¹R² and NR³R⁴, R¹ and R², and R³ and R⁴ each independently may form a substituted ring.

In the above formula, where X is NR¹R² and U has the formula NR¹NR¹—(CR¹₂)ₙ—NR³R⁴ (2), R¹ and R² in NR¹R², and R³ and R⁴ in NR³R⁴ each may independently form a substituted ring containing one or more N, O or S. For example, X is optionally substituted with amino, carbamate, a C₁₋₁₀ alkyl containing one or more non-adjacent N, O or S, and optionally substituted with a heterocyclic ring; aryl or a saturated or unsaturated heterocyclic ring, each of which is optionally substituted. In one example, X and NR³R⁴ are independently morpholine, thiomorpholine, imidazole, pyrrolidine, piperazine, pyridine or piperidine. In one example, X and NR³R⁴ are independently pyrrolidine. In another example, X is pyrrolidine substituted with pyrazine. In this example, V is H; A is fluoro; and W is naphthalenyl. Examples of 5-6 membered heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, and 2,3,4,4a,9,9a-hexahydro-1H-β-carboline.

In the above formula I, W may be benzene, pyridine, biphenyl, naphthalene, phenanthrene, quinoline, isoquinoline, quinazoline, cinnoline, phthalazine, quinoxaline, indole, benzimidazole, benzoxazole, benzthiazole, benzofuran, anthrone, xanthone, acridone, fluorenone, carbazolyl, pyrimido[4,3-b]furan, pyrido[4,3-b]indole, pyrido[2,3-b]indole, dibenzofuran, acridine or acridizine.

In the above formula I, each optionally substituted moiety is substituted with one or more halo, OR², NR¹R², carbamate, C₁₋₁₀ alkyl, C₂₋₁₀ alkenyl, each optionally substituted by halo, ═O, aryl or one or more heteroatoms; inorganic substituents, aryl, carbocyclic or a heterocyclic ring.

The compounds of the present invention may be chiral. As used herein, a chiral compound is a compound that is different from its mirror image, and has an enantiomer. Methods of synthesizing chiral compounds and resolving a racemic mixture of enantiomers are well known to those skilled in the art. See, e.g., March, “Advanced Organic Chemistry,” John Wiley and Sons, Inc., New York, (1985), which is incorporated herein by reference. Where the compound is chiral, the invention includes each individual isomer of the compound as well as mixtures of isomers, such as racemic mixtures and mixtures of diastereomers.

In a second aspect, the invention includes a compound of this formula (II):

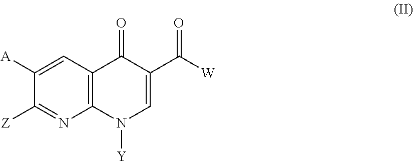

and pharmaceutically acceptable salts, esters and prodrugs thereof;

wherein W is NR¹OR² or NR¹NR¹R² wherein R¹ and R² together with N may form an optionally substituted ring;

Z is NR¹R², —NR¹—NR¹R², —NR¹—OR² or an optionally substituted seven-membered carbocyclic or azacyclic ring, wherein R¹ and R² together with N may form an optionally substituted ring;

A is H, halo or NR¹₂;

each R¹ is independently H or a C₁₋₆ alkyl;

each R² is independently H or a C₁₋₁₀ alkyl or C₂₋₁₀ alkenyl optionally containing one or more non-adjacent heteroatoms selected from N, O, and S, and is optionally substituted with an optionally substituted carbocyclic or heterocyclic ring;

or R² is an optionally substituted heterocyclic ring, aryl or heteroaryl ring;

Y is selected from the group consisting of

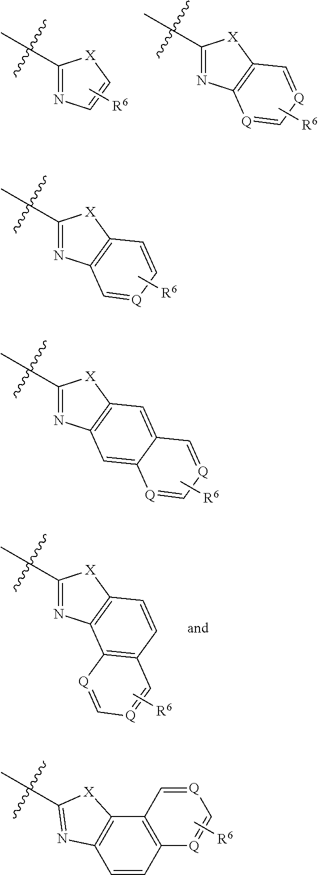

where R⁶ is a substituent at any position on the fused ring; and is H, OR¹, C₁₋₆ alkyl, C₂₋₆ alkenyl, each optionally substituted by halo, ═O or one or more heteroatoms; or R⁵ is an inorganic substituent; or two adjacent R⁶ is linked to obtain a 5-6 membered substituted or unsubstituted carbocyclic or heterocyclic ring, optionally fused to an additional substituted or unsubstituted carbocyclic or heterocyclic ring;
Q is CH or N;
and X is O, NH, or S.

In the above formula II, A may be halo. In one example, A is fluoro.

In the above formula II, Y may have the formula

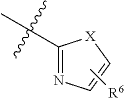

where X is S and R₆ is H;
or the formula

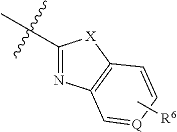

where X is S, Q is CH, and R₆ is H.

In the above formula II, W can be —NR¹NR¹R² or —NR¹OR². In one example, R¹ is H and R² is a C₁₋₁₀ alkyl optionally containing one or more heteroatoms, and optionally substituted with a C₃₋₆ cycloalkyl, aryl or a 5-14 membered heterocyclic ring containing one or more N, O or S, which rings are optionally substituted. In another example, R¹ is H and R² is an aryl or a 5-14 membered heterocyclic ring containing one or more N, O or S, each optionally substituted with an amino or another heterocyclic ring. In yet another example, R¹ and R² in NR¹R² form an optionally substituted 5-14 membered ring containing one or more N, O or S. In particular examples, NR¹R² is morpholine, thiomorpholine, piperazine, piperidine or diazepine.

In other embodiments, W is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. In other embodiments, NR¹R² of U is an acyclic group, wherein R¹ is H or an optionally substituted C₁-C₆ alkyl such as methyl or ethyl or methoxymethyl or trifluoroethyl, and R² represents a ring such as pyridine, phenyl, pyrazine, piperidine, pyrrolidine, pyrrole, morpholine, piperazine, imidazole, imidazolidine, triazine, pyrazole, and the like.

In the above formula 1, W may have the formula

NR¹NR¹—(CR¹₂)ₙ—NR³R⁴ or NR¹O—(CR¹₂)ₙ—NR³R⁴ (2)

wherein R¹ and R³ are independently H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR³R⁴, R³ and R⁴ may form an optionally substituted ring.

In the above formula II, Z can be NR¹R². In one example, R¹ is H and R² is a C₁₋₁₀ alkyl optionally containing one or more heteroatoms, and optionally substituted with a C₃₋₆ cycloalkyl, aryl or a 5-14 membered heterocyclic ring containing one or more N, O or S, which rings are optionally substituted. In another example, R¹ is H and R² is an aryl or a 5-14 membered heterocyclic ring containing one or more N, O or S, each optionally substituted with an amino or another heterocyclic ring. In yet another example, R¹ and R² in NR¹R² form an optionally substituted 5-14 membered ring containing one or more N, O or S. In particular examples, NR¹R² is morpholine, thiomorpholine, piperazine, piperidine or diazepine.

In the above formula 1, Z may have the formula

NR¹—(CR¹₂)ₙ—NR³R⁴ (2)

wherein R¹ and R³ are independently H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR³R⁴, R³ and R⁴ may form an optionally substituted ring.

In the above formula (2), n may be 2-3. In one example, NR³R⁴ is an acyclic amine, or guanidinyl or a tautomer thereof; or R³ and R⁴ optionally form a substituted ring containing one or more N, O or S. In particular examples, NR³R⁴ is morpholine, thiomorpholine, imidazole, pyrrolidine, piperazine, pyridine or piperidine.

In the above formula II, Z may be NR¹R²; and W may have the formula

NR¹NR¹—(CR¹₂)—NR³R⁴ (2)

wherein R¹ and R² are as defined in claim 1;

R³ is H or C₁₋₆ alkyl;

n is 1-6; and

R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and

wherein in NR¹R² and NR³R⁴, R¹ and R², and R³ and R⁴ each independently may form a substituted ring.

In the above formula (2), where Z is NR¹R²; and W has the formula NR¹NR¹—(CR¹₂)ₙ—NR³R⁴ (2), R¹ and R² in NR¹R², and R³ and R⁴ in NR³R⁴ each may independently form a substituted ring containing one or more N, O or S. For example, Z is optionally substituted with amino, carbamate, a C₁₋₁₀ alkyl containing one or more non-adjacent N, O or S, and optionally substituted with a heterocyclic ring; aryl or a saturated or unsaturated heterocyclic ring, each of which is optionally substituted. In one example, Z and NR³R⁴ are independently morpholine, thiomorpholine, imidazole, pyrrolidine, piperazine, pyridine or piperidine. In one example, Z and NR³R⁴ are independently pyrrolidine. In another example, Z is pyrrolidine substituted with pyrazine.

Examples of 5-6 membered heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, and 2,3,4,4a,9,9a-hexahydro-1H-β-carboline.

In the above formula II, Z may be OR² and R² is a C₁₋₆ alkyl optionally substituted with a carbocyclic or heterocyclic ring.

In the above formula II, each optionally substituted moiety is substituted with one or more halo, OR², NR¹R², carbamate, C₁₋₁₀ alkyl, C₂₋₁₀ alkenyl, each optionally substituted by halo, ═O, aryl or one or more heteroatoms; inorganic substituents, aryl, carbocyclic or a heterocyclic ring.

In another aspect, the invention includes a compound of formula (III) or (IV):

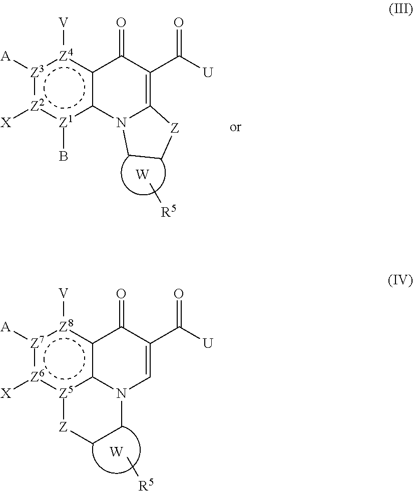

A, V, and X are independently H, halo, azido, R², CH₂R², SR², OR², —NR¹—NR¹R², —NR¹—OR², NR¹R² or an optionally substituted seven-membered carbocyclic or azacyclic ring; or

wherein A and X, or A and V may form a carbocyclic ring, heterocyclic ring, aryl or heteroaryl, each of which may be optionally substituted and/or fused with a cyclic ring;

wherein in NR¹R², R¹ and R² together with N may form an optionally substituted ring;

each Z is CH₂, O, S, or NR¹;

Z¹, Z², Z³, Z⁴, Z⁶, Z⁷, and Z⁸ are independently C or N;

Z⁵ is C or N, provided Z⁵ is C if Z is O, S or NR¹, and further provided that Z and Z⁶ are not N if Z⁵ is N;

U is NR¹NR¹R² or NR¹OR²;

each R¹ is independently H or a C₁₋₆ alkyl;

each R² is independently H or a C₁₋₁₀ alkyl or C₂₋₁₀ alkenyl optionally containing one or more non-adjacent heteroatoms selected from N, O, and S, and optionally substituted with a carbocyclic or heterocyclic ring, which is itself optionally substituted; or R² is an optionally substituted carbocyclic ring, heterocyclic ring, aryl or heteroaryl;

each B is H or halo;

each W is an optionally substituted aryl or heteroaryl, which may be monocyclic, or fused with a single or multiple ring and optionally containing a heteroatom;

and each R⁵ is a substituent at any position on the fused ring; and is H, OR², NR¹R², C₁₋₆ alkyl, C₂₋₆ alkenyl, each optionally substituted by halo, C═O or one or more heteroatoms; or R⁵ is an inorganic substituent; or two adjacent R⁵ is linked to obtain a 5-6 membered substituted or unsubstituted carbocyclic or heterocyclic ring, optionally fused to an additional substituted or unsubstituted carbocyclic or heterocyclic ring.

In some preferred embodiments, U is NR¹NR¹—(CH₂)ₙNR³R⁴, wherein n is 2-3, and wherein R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring, which is also optionally substituted; and wherein in NR³R⁴, R³ and R⁴ may form an optionally substituted ring such as those previously described above. In some such embodiments, at least one of Z¹, Z², Z³, and Z⁴ is N; for example, Z¹ is N and Z²-Z⁴ are each C. In such embodiments, B is absent, and A can be H or halo, preferably Cl or F. Z in these embodiments can be NH, NR¹, O or S, and in some embodiments it is S or NH or NMe. X in these embodiments is typically an aryl or heterocyclic or heteroaryl ring, but may also be an optionally substituted alkylamine group, such as dimethylamine, ethylamine, methoxyethylamine, 2-fluoroethylamine, dimethylaminoethylamine; or, for example, X can be 4-morpholinyl, 1-piperidinyl, 1-pyrrolidinyl, 1-piperazinyl, 4-methyl-1-piperazinyl, or it can be an amine substituted by any one of these rings such as 4-methyl-1-piperazinylamino or pyrrolidin-1-ylamino; or X can be a benzylamino group such as 4-methoxybenzylamine or halobenzylamine, or it can be an aryl such as an optionally substituted phenyl group, e.g., 4-hydroxyphenyl or 4-dimethylaminophenyl; or X can be a heteroaryl-containing group such as pyrazinylamine, pyrazinylamino-amine, pyrazinylmethylamine, or pyrazinylmethyl(methyl)amine; or a corresponding 2-pyridyl, 3-pyridyl or 4-pyridyl analog of one of these pyrazine-containing groups.

In other embodiments, U is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. Exemplary embodiments of U include NH—NR¹R², where NR¹R² can be an optionally substituted cyclic group; examples include 1-piperidinyl, 4-morpholinyl, dimethylamino, 1-pyrrolidinyl, 4-methyl-1-piperazinyl, 1-piperazinyl, 2-pyridylamino, 1,2,4-triazol-4-yl, imidazolidin-2-yl-amino, imidazolidin-2-yl-(methyl)amino, 1-methyl-imidazolidin-2-yl-amino, 2-methoxymethyl-1-pyrrolidinyl, and the like. In other embodiments, NR¹R² of U is an acyclic group, wherein R¹ is H or an optionally substituted C₁-C₆ alkyl such as methyl or ethyl or methoxymethyl or trifluoroethyl or 2-fluoroethyl, and R² represents a ring such as pyridine, phenyl, pyrazine, piperidine, pyrrolidine, pyrrole, morpholine, piperazine, imidazole, imidazolidine, triazine, pyrazole, and the like. Z¹, Z², Z³, Z⁴, Z⁶, Z⁷, and Z⁸ are independently C or N; in some embodiments, no two of these that are adjacent are simultaneously N (i.e., if two of them represent N, the two N are preferably non-adjacent);

In some such embodiments, at least one of Z¹, Z², Z³, and Z⁴ is N; for example, Z¹ is N and Z²-Z⁴ are each C. In such embodiments, B is absent, and A can be H or halo, preferably Cl or F. Z in these embodiments can be NH, NR¹, O or S, and in some embodiments it is S or NH or NMe. X in these embodiments is typically an aryl or heterocyclic or heteroaryl ring, but may also be an optionally substituted alkylamine group, such as dimethylamine, ethylamine, methoxyethylamine, 2-fluoroethylamine, dimethylaminoethylamine; or, for example, X can be 4-morpholinyl, 1-piperidinyl, 1-pyrrolidinyl, 1-piperazinyl, 4-methyl-1-piperazinyl, or it can be an amine substituted by any one of these rings such as 4-methyl-1-piperazinylamino or pyrrolidin-1-ylamino; or X can be a benzylamino group such as 4-methoxybenzylamine or halobenzylamine, or it can be an aryl such as an optionally substituted phenyl group, e.g., 4-hydroxyphenyl or 4-dimethylaminophenyl; or X can be a heteroaryl-containing group such as pyrazinylamine, pyrazinylamino-amine, pyrazinylmethylamine, or pyrazinylmethyl(methyl)amine; or a corresponding 2-pyridyl, 3-pyridyl or 4-pyridyl analog of one of these pyrazine-containing groups.

In some embodiments of the compound of formula (III), one of Z¹-Z⁴ is N and the others each represent C. In some such embodiments, Z¹ is N, in others Z² is N, in others Z³ is N and in others, Z⁴ is N.

Z in some embodiments is O, S, or NR¹. In many embodiments, Z is selected from O, S, NH, and NMe.

W if present in any of the compounds of formula (III) or (IV) may be selected from the group consisting of

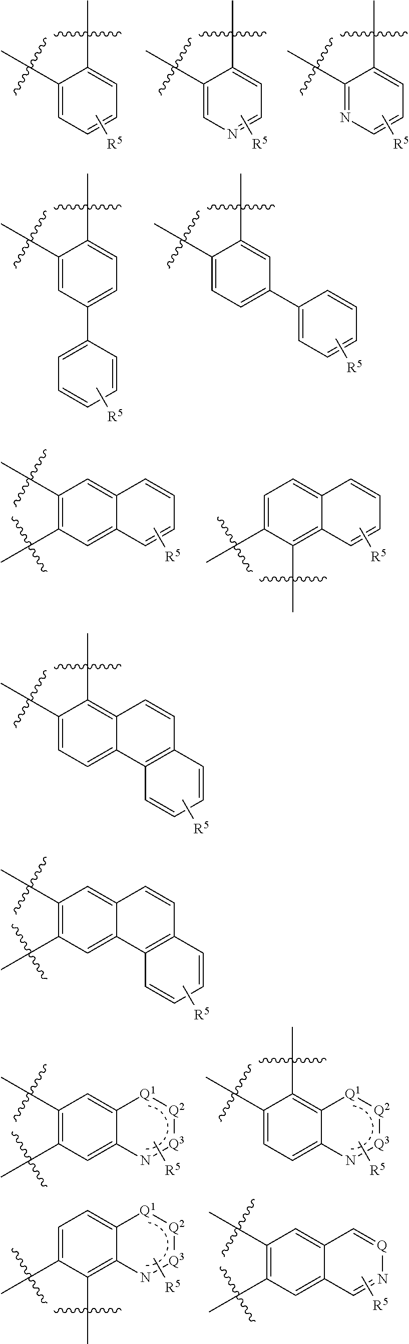

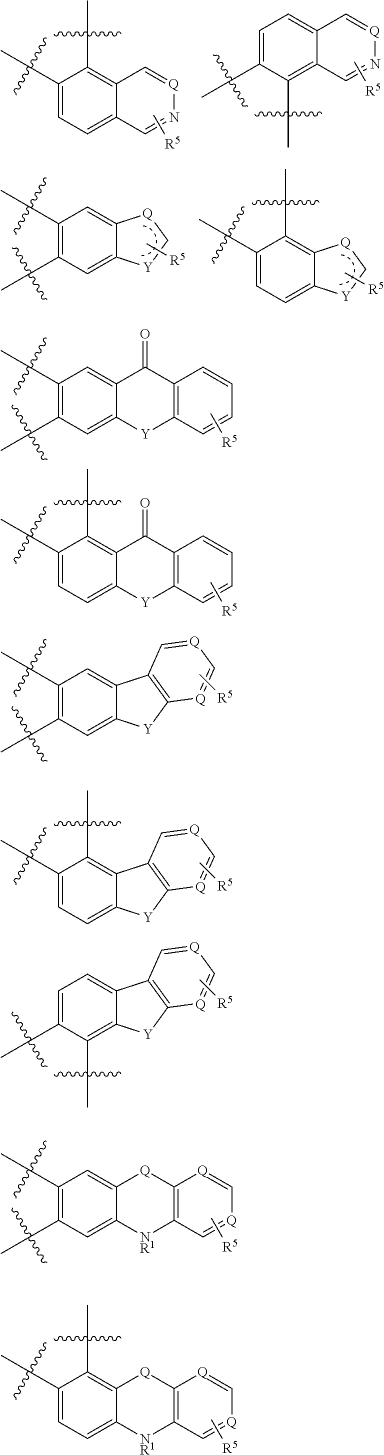

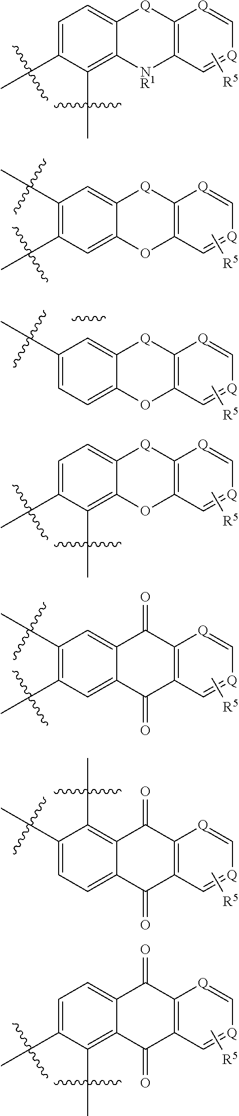

wherein each Q, Q¹, Q², and Q³ is independently CH or N;
Y is independently O, CH, C═O or NR¹;
and R⁵ is as defined above.

In the above methods, X in any of the above compounds may be SR², where R² is a C₀₋₁₀ alkyl or C₂₋₁₀ alkenyl optionally substituted with a heteroatom, a carbocyclic ring, a heterocyclic ring, an aryl or a heteroaryl. In one example, R² is a C₁₋₁₀ alkyl substituted with an optionally substituted pyrazine.

In the above methods, X and A in any of the above compounds may independently be halo or NR¹R², wherein R¹ may be H and R² may be a C₁₋₁₀ alkyl optionally substituted with a heteroatom, a C₃₋₆ cycloalkyl, aryl or a 5-14 membered heterocyclic ring containing one or more N, O or S. In one example, R² is a C₁₋₁₀ alkyl substituted with an optionally substituted heterocyclic ring, particularly a 5-14 membered heterocyclic ring. Alternatively, R¹ and R² together with N may form an optionally substituted heterocyclic ring containing one or more N, O or S, and particularly a 5-14 membered heterocyclic ring.

Examples of optionally substituted heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, aminodithiadazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, and 2,3,4,4a,9,9a-hexahydro-1H-β-carboline.

In the above methods, A and X in any of the above compounds may independently be halo or NR¹R², where R¹ is H and R² is a C₁₋₁₀ alkyl substituted with morpholine, thiomorpholine, imidazole, aminodithiadazole, pyrrolidine, piperazine, pyridine or piperidine.

Alternatively, R¹ and R² together with N may form with N piperidine, pyrrolidine, piperazine, morpholine, thiomorpholine, imidazole, or aminodiathiazole.

Alternatively, X in any of the above compounds may be an amine moiety having the formula

NR¹—(CR¹₂)ₙ—NR³R⁴ (3)

wherein R¹ and R³ are independently H or C₁₋₆ alkyl;
n is 1-6; and
R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and
wherein in NR³R⁴, R³ and R⁴ may form an optionally substituted ring.

In the above amine moiety of formula (3), n may be 2-3. In one example, R³ and R⁴ in NR³R⁴ together form an optionally substituted ring containing one or more N, O or S.

In some embodiments, A, V, B and X, when present, are all H. In other embodiments, three of A, V, B and X are H. In other embodiments, two of A, V, B and X are H. In yet other embodiments, only one of A, V, B and X is H. In certain embodiments, only one of A, V, B and X is a halogen (e.g., fluorine). In other embodiments, two of A, V, B and X are halogen. In still other embodiments, three of A, V, B and X are halogen.

In the above embodiments, each optionally substituted moiety in any of the above compounds may be substituted with one or more halo, OR², NR¹R², carbamate, C₁₋₁₀ alkyl, C₂₋₁₀ alkenyl, each optionally substituted by halo, C═O, aryl or one or more heteroatoms; inorganic substituents, aryl, carbocyclic or a heterocyclic ring.

In certain embodiments, a compound of formula (III) or (IV) may have some combination of the following features:

each of A, V and B is independently H or halogen (e.g., chloro or fluoro);

X is —NR¹R² or CR¹R², wherein, R¹ and R² together may form an optionally substituted heterocyclic, aryl or heteroaryl ring;

Z is S or NH or N-alkyl (e.g., N—CH₃);

W is an optionally substituted aryl or heteroaryl ring; and

R² of U is —(CH₂)ₘ—NR³R⁴, wherein m is 2-4, and R³ and R⁴ together with N may form an optionally substituted heterocyclic or heteroaryl ring.

In certain embodiments, the compounds of formula (III) have a phenyl group or a napthylene group for W. In some such embodiments, at least one of Z¹, Z², Z³, and Z⁴ is N; for example, Z¹ is N and Z²-Z⁴ are each C. In such embodiments, B is absent, and A can be H or halo, preferably Cl or F, while V is typically H or halo. U in these embodiments can be NH—NR¹R², where NR¹R² is an optionally substituted cyclic group; examples include 1-piperidinyl, 4-morpholinyl, dimethylamino, 1-pyrrolidinyl, 4-methyl-1-piperazinyl, 1-piperazinyl, 2-pyridylamino, 1,2,4-triazol-4-yl, imidazolidin-2-yl-amino, imidazolidin-2-yl-(methyl)amino, 1-methyl-imidazolidin-2-yl-amino, 2-methoxymethyl-1-pyrrolidinyl, and the like. In other embodiments, NR¹R² of U is an acyclic group, wherein R¹ is H or an optionally substituted C₁-C₆ alkyl such as methyl or ethyl or methoxymethyl or trifluoroethyl or 2-fluoroethyl, and R² represents a ring such as pyridine, phenyl, pyrazine, piperidine, pyrrolidine, pyrrole, morpholine, piperazine, imidazole, imidazolidine, triazine, pyrazole, and the like. Z in these embodiments can be NH, NR¹, O or S, and in some embodiments it is S or NH or NMe. X in these embodiments is typically an aryl or heterocyclic or heteroaryl ring, but may also be an optionally substituted alkylamine group, such as dimethylamine, ethylamine, methoxyethylamine, 2-fluoroethylamine, dimethylaminoethylamine; or, for example, X can be 4-morpholinyl, 1-piperidinyl, 1-pyrrolidinyl, 1-piperazinyl, 4-methyl-1-piperazinyl, or it can be an amine substituted by any one of these rings such as 4-methyl-1-piperazinylamino or pyrrolidin-1-ylamino; or X can be a benzylamino group such as 4-methoxybenzylamine or halobenzylamine, or it can be an aryl such as an optionally substituted phenyl group, e.g., 4-hydroxyphenyl or 4-dimethylaminophenyl; or X can be a heteroaryl-containing group such as pyrazinylamine, pyrazinylamino-amine, pyrazinylmethylamine, or pyrazinylmethyl(methyl)amine; or a corresponding 2-pyridyl, 3-pyridyl or 4-pyridyl analog of one of these pyrazine-containing groups.

In another aspect, the invention includes a compound of this formula:

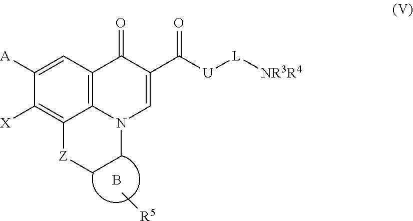

and pharmaceutically acceptable salts, esters and prodrugs thereof, wherein:
A is H or F;
X is H, halo, —NR¹—NR¹R², —NR¹—OR², NR¹R² or an optionally substituted seven-membered carbocyclic or azacyclic ring;
Z is O, S, NR¹ or CH₂;
U is NR¹NR¹ or U can be NR¹—O in which L is attached to O;
L is a C₁₋₁₀ alkylene group, optionally containing one heteroatom selected from N, O and S, and optionally substituted;
B is 5-6 membered aryl or heteroaryl, which is optionally substituted;
R¹ and R³ are independently H or a C₁₋₆ alkyl;
R² and R⁴ is H, or a C₁₋₁₀ alkyl or C₂₋₁₀ alkenyl optionally containing one or more non-adjacent heteroatoms selected from N, O, and S, and optionally substituted with a substituted or unsubstituted aryl, heteroaryl, carbocyclic, or heterocyclic ring; or R² is an optionally substituted cycloalkyl, heterocyclic ring, aryl or heteroaryl;
and wherein R¹ and R², and/or R³ and R⁴, together with N to which both are attached, can form a 5-14 membered heterocyclic or heteroaromatic ring, which is optionally substituted;
R⁵ is a substituent at any position of W and is H, halo, cyano, —CONHR², OR², or C₁₋₆ alkyl or C₂₋₆ alkenyl, each optionally substituted by halo, ═O or one or more heteroatoms;
wherein each optionally substituted moiety is substituted with one or more halo, cyano, azido, acetyl, amido, OR², NR¹R², carbamate, C₁₋₁₀ alkyl, C₂₋₁₀ alkenyl, each optionally substituted by halo, ═O, aryl or one or more heteroatoms selected from N, O and S; or is substituted with an aryl, a carbocyclic or a heterocyclic ring.

In the above formula (V), W may be phenyl or pyridyl or naphthyl, each of which can be substituted or unsubstituted. In some embodiments, L is a C₂₋₄ alkylene.

In the above formula (V), X may be NR¹R², and R² is an optionally substituted cyclopropyl, phenyl, or imidazole, or a C₁₋₆ alkyl optionally substituted with a cyclopropyl or OR¹.

In some embodiments, the NR¹R² and NR³R⁴ moieties in formula (V) are independently i an optionally substituted morpholine, thiomorpholine, imidazole, pyrrolidine, piperazine, pyridine or piperidine.

In yet other embodiments, A in formula (V) is F and R⁵ is halo, cyano, amido or azido.

In another aspect, the invention provides a compound of one of these formulas:

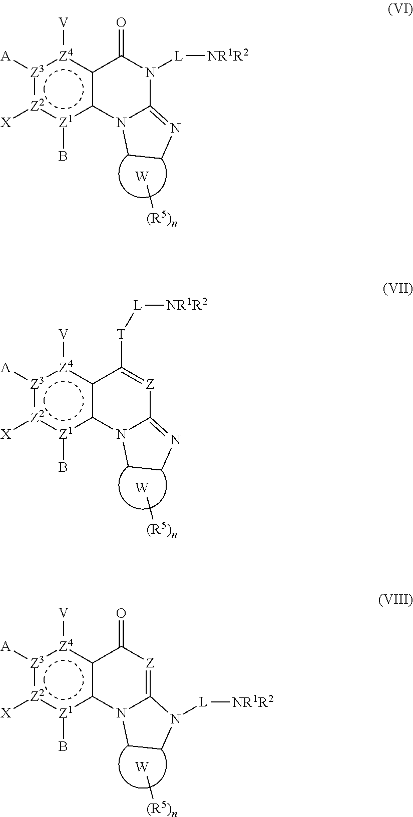

wherein each A, V, B, and X that is present is independently selected from H, halo, azido, CN, CF₃, CONR¹R², R², —NR¹—NR¹R², —NR¹—OR², CH₂R², SR², OR²C(═O)R², an optionally substituted seven-membered carbocyclic or azacyclic ring, and NR¹R², wherein in NR¹R², R¹ and R² can optionally cyclize to form an optionally substituted azacyclic group; or

wherein A and X, or A and V, or X and B may form a carbocyclic ring, heterocyclic ring, aryl or heteroaryl ring, each of which may be optionally substituted with one or two R³ groups and/or may be fused with an additional ring;

wherein in L-NR¹R², R¹ and R² taken together may form an optionally substituted azacyclic group, or R¹ or R² taken together with at least a portion of L may form an optionally substituted heterocyclic ring;

each Z is independently CH, CR³ or N;

each Z¹, Z², Z³, and Z⁴ is independently C or N, provided no two of them represent adjacent nitrogen atoms;

T is C(O)NR⁴, S(O)ₘNR⁴ or NR⁴;

each R¹ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each m is independently 0-2;

each n is independently 0-4;

each R² is independently H or an optionally substituted C₁₋₁₀ alkyl or optionally substituted C₂₋₁₀ alkenyl optionally containing one or more non-adjacent heteroatoms selected from N, O, and S in place of carbon atoms, and optionally including a carbocyclic or heterocyclic ring; or R² is an optionally substituted carbocyclic, heterocyclic, 6-10 membered aryl or 5-14 membered heteroaryl ring containing one or more N, O or S;

each R³ is independently an optionally substituted group selected from C₁₋₆ alkyl, C₆₋₁₀ aryl, and C₅₋₁₂ heteroaryl, or R³ is selected from halo, nitro, OR′, SR′, SO₂R′, NR′₂, CN, CF₃, COOR′, and CONR′₂, wherein each R′ is independently H or C₁₋₆ alkyl and can optionally include one N, O or S in place of a carbon atom, or R³ can be L-NR¹R² or CON(R′)-L-NR¹R², wherein in NR¹R², R¹ and R² can optionally cyclize to form an optionally substituted azacyclic group;

each R⁴ is H or a C₁₋₁₀ alkyl or C₂₋₁₀ alkenyl group that can be substituted with one or more substituents selected from halo, ═O, OR², NR²₂, S(O)ₘR², COOR², and CONR²₂;

each L is —NR¹(CH₂)ₘ— or —O(CH₂)ₘ—, where m is 1-6, and preferably m is 2-3;

each W represents an optionally substituted aryl or heteroaryl ring, which may be a monocyclic group with 5-6 ring atoms, or may be a 5-6 membered ring that is fused with or bonded to one or more additional aryl, heterocyclic, or heteroaryl rings; and

each R⁵ is a substituent at any position on W, and is selected from H, halo, CN, CF₃, OR², NR¹R², and C₁₋₆ alkyl and C₂₋₆ alkenyl, each optionally substituted by one or more substituents selected from halo, ═O, OR², S(O)ₘR², and NR¹R², wherein in NR¹R², R¹ and R² can optionally cyclize to form an optionally substituted azacyclic group; or R⁵ can be an inorganic substituent; or two adjacent R⁵ may be linked to form a 5-6 membered substituted or unsubstituted carbocyclic or heterocyclic ring, optionally fused to an additional substituted or unsubstituted carbocyclic or heterocyclic ring;

or a pharmaceutically acceptable salt thereof.

In the above formulas (VI), (VII), and (VIII), the five-membered ring containing two nitrogens is an imidazole or imidazoline derivative, and W represents a ring that includes two ring atoms of the imidazole/imidazoline ring shown. W is typically an optionally substituted 5- or 6-membered aromatic or heteroaromatic ring that is optionally fused to another substituted aryl or heteroaryl or heterocyclic or carbocyclic group. Frequently, W is selected from the group consisting of the following structures, in which the open valences (broken bonds) indicate where W attaches to each of the N atoms of the imidazole/imidazoline ring in formula (VI), (VII), and (VIII):

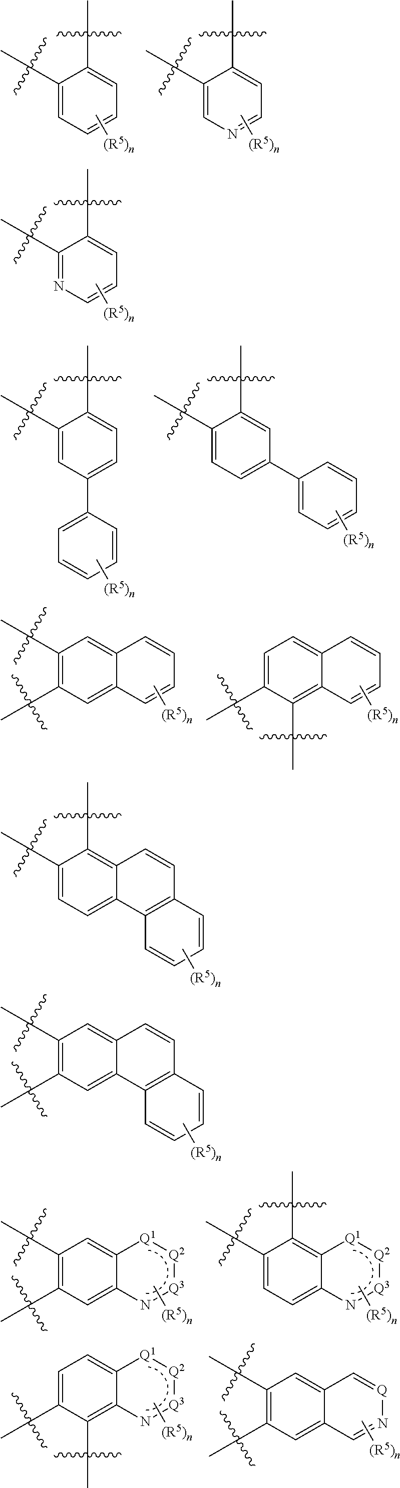

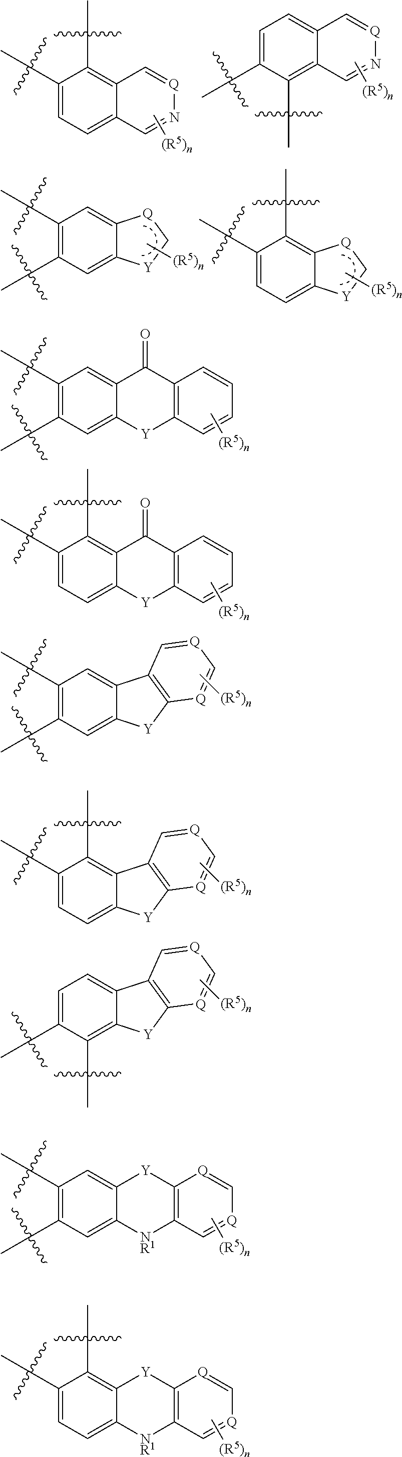

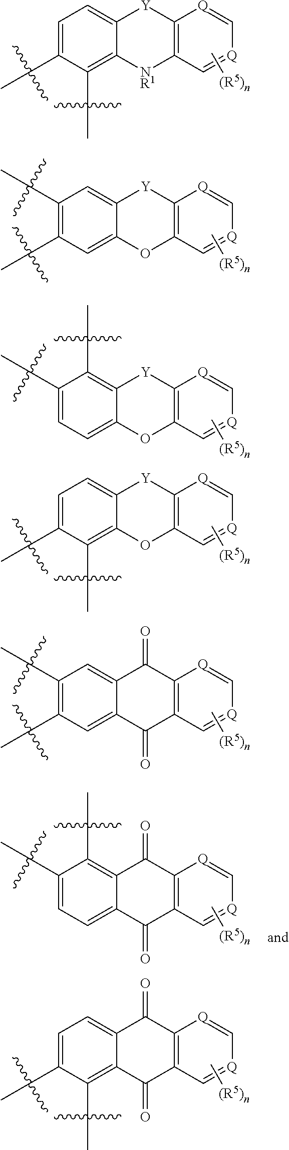

wherein each Q, Q¹, Q², and Q³ is independently CR³ or N;
each Y is independently O, CR¹₂, C═O or NR¹; and
n, R¹, R³ and R⁵ are as defined above.

In the multiple-ring structures above that represent specific embodiments of W, (R⁵)ₙ is depicted as though it is positioned on one particular ring even though the embodiment of W comprises multiple rings; that is for convenience of drawing, only, though, and it is understood that substituents represented by R⁵ may be on any available valence of any of the rings comprising W.

The compounds of formulas (VI), (VII), and (VIII) are characterized by a polycyclic core and an essential amine group represented by -L-NR¹R². The amine group -L-NR¹R² can be attached at one of three positions as represented by the three formulas, and in some embodiments, the compounds of the invention include a second -L-NR¹R² group as well, which can be attached to Z in formula (VII) or formula (VIII) when that Z represents C, for example.

The NR¹R² portion of the -L-NR¹R² group is often a basic amine group; for example, it can be a dialkyl amine such as dimethyl amine or diethyl amine, or it can be a cyclic group such as morpholine, piperidine, pyrrolidine, aziridine, azetidine, azepine, or piperazine when R¹ and R² are linked together. In each case, R¹ and R² of this L-NR¹R² group can be substituted with substituents including those described below. Preferably, the substituents on -L-NR¹R² do not include a carbonyl oxygen on any carbon atom that is directly linked to N.

R¹ or R² can cyclize onto L or a portion of L to form an optionally substituted heterocyclic ring having 3-8 ring members and optionally one additional heteroatom selected from N, O and S as a ring member in addition to the nitrogen of the NR¹R² group; or R¹ and R² can cyclize together to form an optionally substituted azacyclic group. In certain embodiments of the above molecules, NR¹R² represents pyrrolidine in at least one -L-NR¹R² group. In specific embodiments, -L-NR¹R² represents a group selected from:

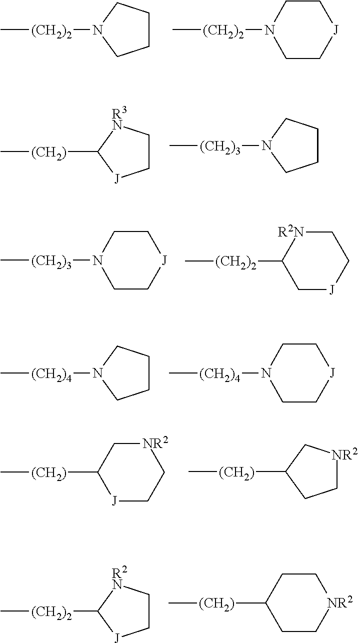

where each J independently represents CH₂, O, S(O)ₘ (wherein m=0-2), NR⁶, NC(O)R⁶, NC(O)OR⁶, NC(O)N(R⁶)₂, or NSO₂R⁶, where each R⁶ is H or a C₁₋₁₀ alkyl that can be substituted with one or more groups selected from halo, ═O, OR′, NR′₂, S(O)ₘR′, COOR′, and CONR′₂, where each R′ is H or C1-C4 alkyl optionally substituted with one or more halo or ═O, and m=0-2; and R² is as defined above. In certain embodiments, m is 2. In some embodiments, R⁶ is a C1-C4 alkyl that can be substituted with halo, ═O, OR′, NR′₂, S(O)ₘR′, S(O)ₘNR′₂, COOR′, or CONR′₂, where each R′ is H or C1-C4 alkyl optionally substituted with one or more halo or ═O, and m=0-2; in certain embodiments, m is 2.

In the above formulas (VI), (VII), and (VIII), B is absent when Z¹ is N. Similarly, when Z² is N, X is absent; when Z³ is N, A is absent; and when Z⁴ is N, V is absent.

In the above formulas (VII), and (VIII), Z may be N or it may be CR³. When Z is CR³, it is CH or C-L-NR¹R² in certain embodiments.

In some embodiments, Z¹ is N, so B is absent; and Z² is a substituted carbon, so X is not H, while Z³ and Z⁴ are each carbons, and A and V are both H. In some embodiments, at least one of B, X, or A is halo and Z¹, Z², and Z³ are each C. In other embodiments, X and A are not both H when Z² and Z³ are C. In the above formulas (VI), (VII), and (VIII), V may be H in certain embodiments where Z⁴ is C.

In one embodiment, each of Z¹, Z², Z³ and Z⁴ is C. In another embodiment, three of Z¹, Z², Z³ and Z⁴ represent C, and the other represents N. For example, Z¹, Z² and Z³ are C, and Z⁴ is N. Alternatively, Z¹, Z² and Z⁴ are C, and Z³ is N. In other examples, Z¹, Z³ and Z⁴ are C and Z² is N. In yet other examples, Z², Z³ and Z⁴ are C, and Z¹ is N.

In another embodiment, two of Z¹, Z², Z³ and Z⁴ are C, and the other two are non-adjacent nitrogens. For example, Z¹ and Z³ may be C, while Z² and Z⁴ are N. Alternatively, Z¹ and Z³ may be N, while Z² and Z⁴ may be C. In other examples, Z¹ and Z⁴ are N, while Z² and Z³ are C.

In some embodiments, Z¹-Z⁴ are C and each of B, X, A, and V is H. In many embodiments, at least one of B, X, A, and V is H and the corresponding adjacent Z¹-Z⁴ atom is C. For example, any two of B, X, A, and V may be H. In one example, V and B may both be H. In another, B and A are both H, and X is not H. In other examples, any three of B, X, A, and V are H and the corresponding adjacent Z¹-Z⁴ atom is C.

In certain embodiments, one of B, X, A, and V is a halogen (e.g., fluorine) and the corresponding adjacent Z¹-Z⁴ is C. In other embodiments, two of X, A, and V are selected from halogen and SR², wherein R² is as defined above; and each corresponding adjacent Z²-Z⁴ is C. For example, each X and A may be a halogen.

In other examples, each X and A present may be SR², wherein R² is as defined above; in certain embodiments, R² is H or C₁₋₁₀ alkyl substituted with an aryl or heteroaryl group such as phenyl or pyrazine, which aryl or heteroaryl such as phenyl or pyrazine may itself be substituted. In yet other examples, any of B, V, A and X may be an alkynyl such as a propargyl group, a fluorinated alkyl such as CF₃, CH₂CF₃, perfluorinated C₂-C₁₀ alkyls, etc.; cyano, nitro, amides, sulfonyl amides, or carbonyl groups such as COR². Sometimes, at least one of V, A, B, and X is OR², where each R² is as defined above; in certain embodiments, R² is H or C₁₋₁₀ alkyl optionally substituted with an aryl or heteroaryl group such as phenyl or pyrazine, which aryl or heteroaryl group such as phenyl or pyrazine may itself be substituted.

In each of the above formulas, X, V, B, and A if present may independently be NR¹R², wherein R¹ and R² are as defined above, and wherein in any NR¹R² group, R¹ and R² can optionally cyclize to form an azacyclic group. In some embodiments, R¹ is H or C₁₋₁₀ alkyl, and R² is a C₁₋₁₀ alkyl optionally containing a heteroatom selected from N, O and S in place of one carbon atom, a C₃₋₆ cycloalkyl, aryl or a 5-14 membered heterocyclic ring containing one or more N, O or S.

If more than one NR¹R² moiety is present in a compound within the invention, as when both A and R³ comprise NR¹R² in a compound according to any one of the above formulas, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with morpholine, thiomorpholine, imidazole, aminodithiadazole, pyrrolidine, piperazine, pyridine or piperidine. Alternatively, R¹ and R² together with the N to which they are both bonded, may form an optionally substituted heterocyclic ring which may contain one or more additional N, O or S. For example, R¹ and R² together with N may form an azacyclic group selected from piperidine, pyrrolidine, piperazine, morpholine, thiomorpholine, imidazole, and aminodithiazole. Each of these azacyclic groups may be further substituted with, for example, one or more groups selected from halo, azido, ═O, CN, CF₃, CONR′₂, R′, CH₂R′, S(O)ₘR, OR′ C(═O)R′, C(═O)OR′, and NR′₂, where each R′ independently represents H or C1-C4 alkyl, and m=0-2, and where NR′₂ can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR″C(═O)R″, C(═O)OR″, and NR″₂, where each R″ independently represents H or C1-C4 alkyl, and each m is independently 0-2.

In certain embodiments, one of X, A B, and V is NR¹R². In such embodiments, NR¹R² sometimes represents an azacyclic group that may be substituted as described above. For example, X, A B, or V may be a 1-piperazinyl group, wherein the 4-position of the piperazine ring is substituted by C(═O)R″, C(═O)OR″, or by CONR″₂, where each R″ independently represents H or C1-C4 alkyl. In certain such embodiments, Z¹ is N, Z² is C, and X is NR¹R₂, which represents an azacyclic group. In some such embodiments, A and V are present and each represent H, and in some such embodiments X represents a substituted piperazine, and the substituent at position 4 of the piperazine ring is an acyl group such as acetyl.

In one embodiment, the present invention provides compounds having formula (VI), (VII), and (VIII) as described above, wherein:

each of A, V and B if present is independently H or halogen (e.g., chloro or fluoro);
X is —NR¹R², wherein R¹ and R² are as defined above and may be taken together to form an azacyclic group, or one of R¹ and R² may be taken together with at least a portion of L to form an optionally substituted heterocyclic, aryl or heteroaryl ring,
or X can represent an aryl or heteroaryl ring that may be substituted with halo, C₁-C₄ alkyl, C1-C4 haloalkyl, or C1-C4 alkoxy;
Z, if present, is N or CH;
W together with the two carbons of the imidazole/imidazoline ring to which it is fused forms a 5- or 6-membered ring that is further fused with an optionally substituted aryl or heteroaryl ring; and
-L-NR¹R² represents NR¹(CH₂)₂₋₄—NR¹R² or O(CH₂)₂₋₄—NR¹R², wherein NR¹R² represents an azacyclic group selected from piperidine, pyrrolidine, piperazine, morpholine, thiomorpholine, and imidazole, each of which may be substituted with one or more groups selected from halo, azido, ═O, CN, CF₃, CONR′₂, R′, CH₂R′, S(O)ₘR′, OR′ C(═O)R′, C(═O)OR′, and NR′₂, where each R′ independently represents H or C₁-C₄ alkyl, and m=0-2, and where NR′₂ can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR″ C(═O)R″, C(═O)OR″, and NR″₂, where each R″ independently represents H or C1-C4 alkyl, and each m is independently 0-2;
or -L-NR¹R² represents a group of formula (4):

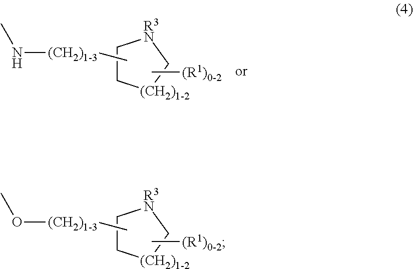

where R¹ and R³ are as defined above, and the optional substituents R¹ and the attachment point for the alkylene linker (CH₂)₁₋₃ can be at any position on the ring other than the nitrogen atom.

In another embodiment, the present invention provides compounds having formula (VI), (VII), and (VIII), wherein:

Z¹ is N, and Z², Z³ and Z⁴ are each C;
A and B are each independently H or halogen (e.g., chloro or fluoro);
X is H or an azacyclic group selected from piperidine, pyrrolidine, piperazine, morpholine, thiomorpholine, imidazole, which may be substituted with one or more substituents selected from halo, azido, ═O, CN, CF₃, CONR′₂, R′, CH₂R′, SR′, OR′ C(═O)R′, and NR′₂, where each R′ independently represents H or C1-C4 alkyl or each NR′₂ can represent an optionally substituted azacyclic group;
Z, if present, is CH or N;
W together with the two carbon atoms of the imidazole/imidazoline ring to which it is fused forms an optionally substituted 5- or 6-membered ring that is further fused with an optionally substituted aryl or heteroaryl ring; and
-L-NR¹R² represents NH—(CH₂)₂₋₄—NR¹R², wherein NR¹R² represents an azacyclic group selected from piperidine, pyrrolidine, piperazine, morpholine, thiomorpholine, and imidazole, each of which may be substituted with halo, azido, ═O, CN, CF₃, CONR′₂, R′, CH₂R′, S(O)ₘR′, OR′ C(═O)R′, C(═O)OR′, and NR′₂, where each R′ independently represents H or C₁-C₄ alkyl, and m=0-2, and where NR′₂ can additionally represent an azacyclic group such as piperidine, pyrrolidine, piperazine, morpholine, thiomorpholine, or imidazole, which azacyclic group can be substituted with one or more halo, azido, ═O, CN, CF₃, CONR″₂, R″, CH₂R″, S(O)ₘR″, OR C(═O)R″, C(═O)OR″, and NR″₂, where each R″ independently represents H or C₁-C₄ alkyl, and each m is independently 0-2;
or -L-NR¹R² represents a group of formula (4) as shown above.

In each of the above formulae, unless otherwise indicated, each optionally substituted moiety may be substituted with one or more halo, OR², NR¹R², carbamate, C₁₋₁₀ alkyl, C₂₋₁₀ alkenyl, each of which is further optionally substituted by halo, ═O, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or OCH₂CF₃, etc.; cyano, nitro, COR², NR²COR², S(O)ₘ, COOR², CONR²₂, OCOR², OCOOR², OCONR²₂, NRCONR²₂, NRC(NR)(NR²₂), NR(CO)NR²₂, and SO₂NR²₂, wherein each R² is as defined in formula 1 and each m is independently 0-2.

In another aspect, the invention provides a compound of this formula:

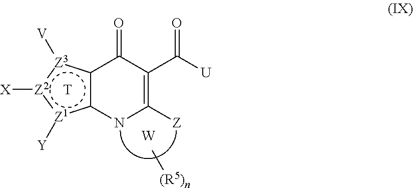

and pharmaceutically acceptable salts, esters and prodrugs thereof;

wherein V, X, and Y are absent if attached to a heteroatom other than Nitrogen, and independently H, halo, azido, R², CH₂R², SR², OR², —NR¹—NR¹R², —NR¹—OR², or NR¹R² when attached to C or N; or

wherein V and X, or X and Y may form a carbocyclic ring, heterocyclic ring, aryl or heteroaryl, each of which may be optionally substituted and/or fused with a cyclic ring;

Z¹, Z² and Z³ are C, N, O or S;

Z is O, S, NR², CH₂ or C═O;

W together with N and Z forms an optionally substituted 5- or 6-membered ring that is fused to an optionally substituted aryl or heteroaryl, wherein said aryl or heteroaryl may be monocyclic or fused with a single or multiple ring, and wherein said ring optionally contains a heteroatom;

U is NR¹NR¹R², NR¹NR¹—(CR¹₂)ₙ—NR³R⁴, NR¹—OR², or NR¹—O—(CR¹₂)ₙ—NR³R⁴;

wherein in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms selected from N, O and S, a carbocyclic ring, a heterocyclic ring, an aryl or heteroaryl, wherein each ring is optionally substituted; or R² is an optionally substituted carbocyclic ring, heterocyclic ring, aryl or heteroaryl; or R² is COR¹ or S(O)ₓR¹ wherein x is 1-2;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on W; and is H, OR², amino, alkoxy, amido, halogen, cyano or an inorganic substituent; or R⁵ is C₁₋₆ alkyl, C₂₋₆ alkenyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring, optionally fused to an additional optionally substituted carbocyclic or heterocyclic ring; and

n is 1-6.

In the above formula (IX), ring T may form an optionally substituted 5-membered ring selected from the group consisting of:

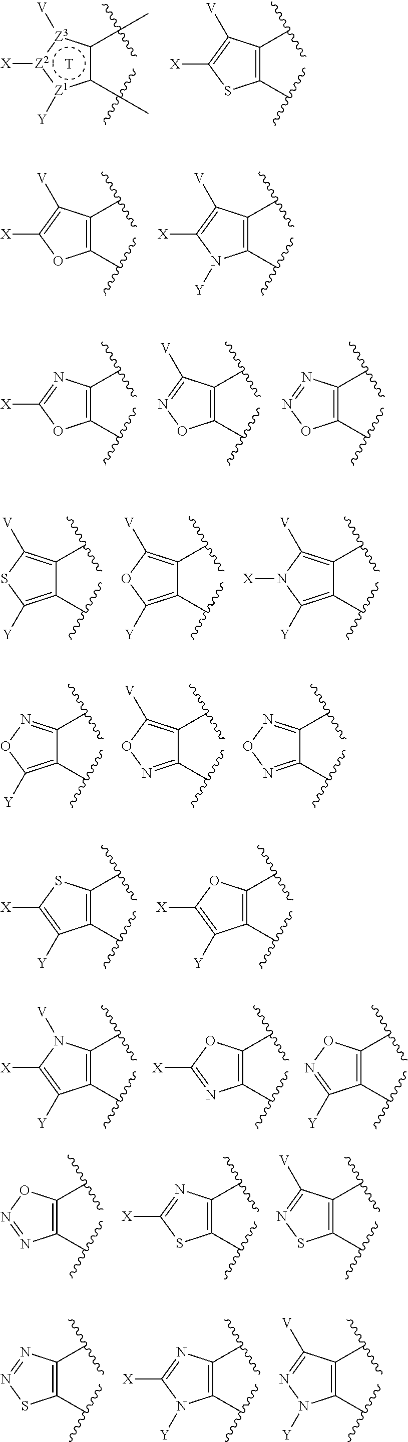

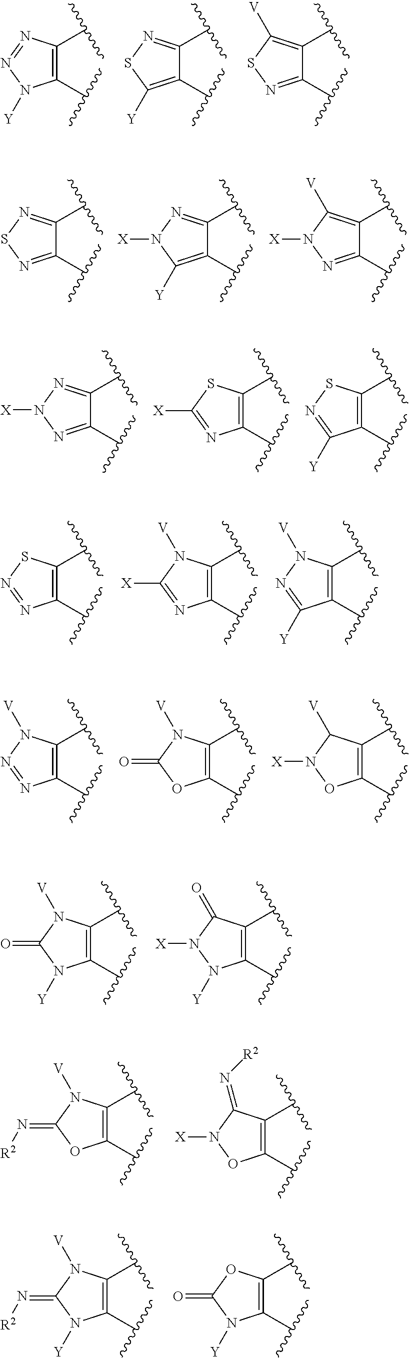

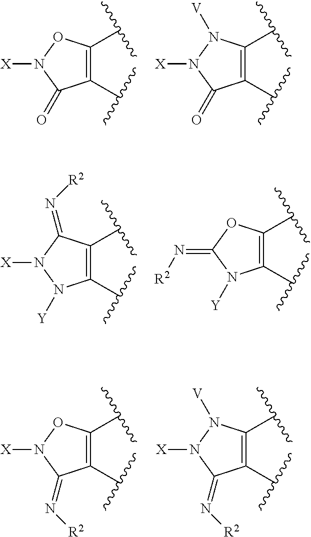

In the above formula (IX), W together with N and Z may form an optionally substituted 5- or 6-membered aryl or heteroaryl ring that is fused to an optionally substituted aryl or heteroaryl selected from the group consisting of:

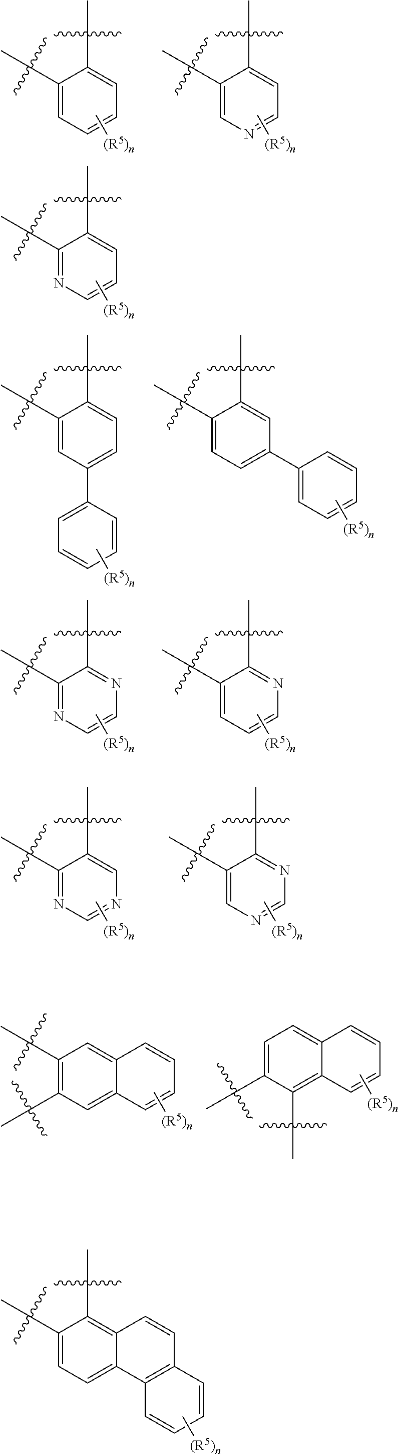

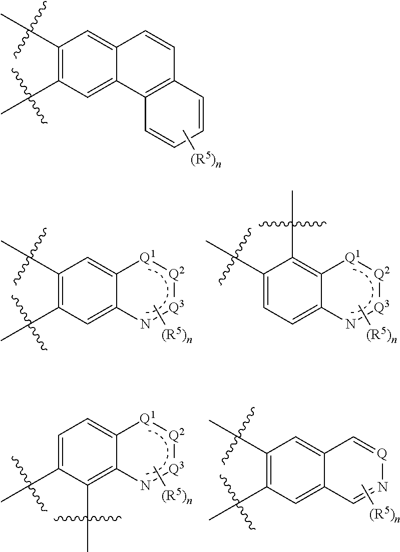

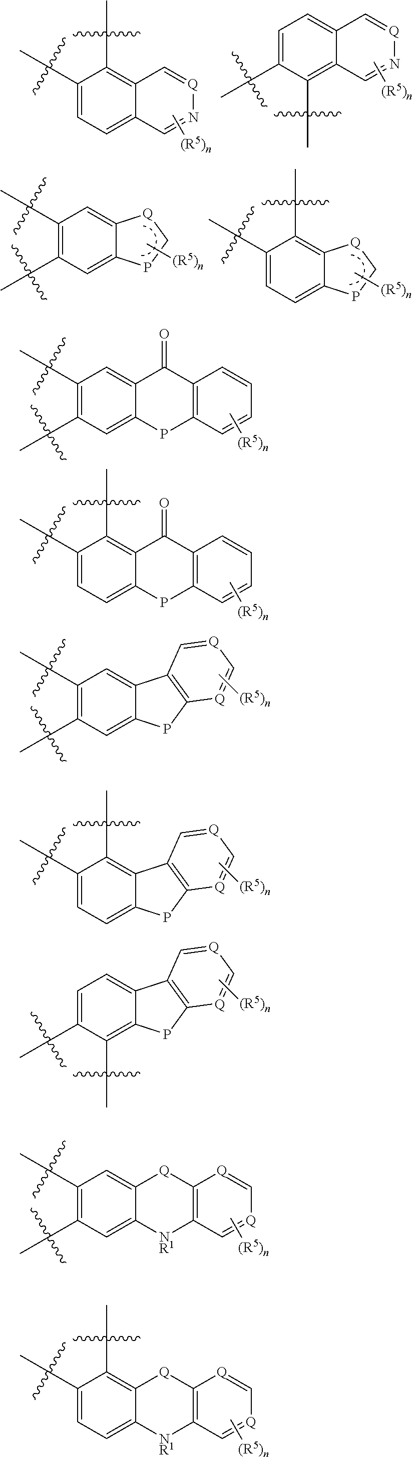

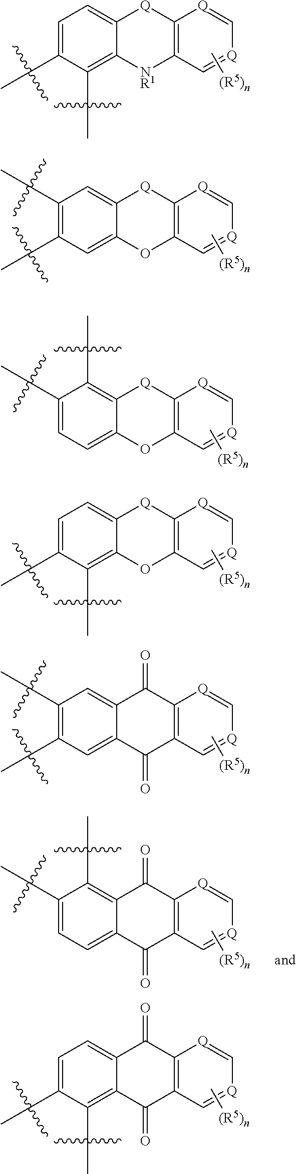

wherein each Q, Q¹, Q², and Q³ is independently CH or N;

P is independently O, CH, C═O or NR¹;

n and R⁵ are as defined above.

In other embodiments, W together with N and Z may form a group having the formula selected from the group consisting of

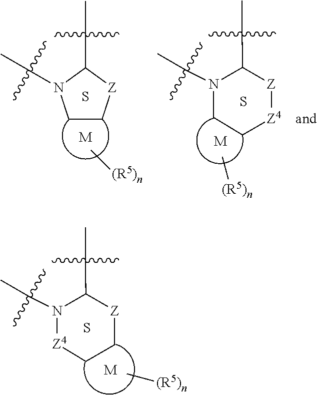

wherein Z is O, S, NR², CH₂ or C═O;

each Z⁴ is CR⁶, NR², or C═O;

R⁶ is H, or a substituent known in the art, including but not limited to hydroxyl, alkyl, alkoxy, halo, amino, or amido; and

Ring S and M may be saturated or unsaturated.

In some embodiments, W together with N and Z may form a 5- or 6-membered ring that is fused to a phenyl.

In some embodiments, U is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. In other embodiments, NR¹R² of U is an acyclic group, wherein R¹ is H or an optionally substituted C₁-C₆ alkyl such as methyl or ethyl or methoxymethyl or trifluoroethyl, and R² represents a ring such as pyridine, phenyl, pyrazine, piperidine, pyrrolidine, pyrrole, morpholine, piperazine, imidazole, imidazolidine, triazine, pyrazole, and the like.

In yet another aspect, the compounds of the present invention have the general formula (X) or (XI):

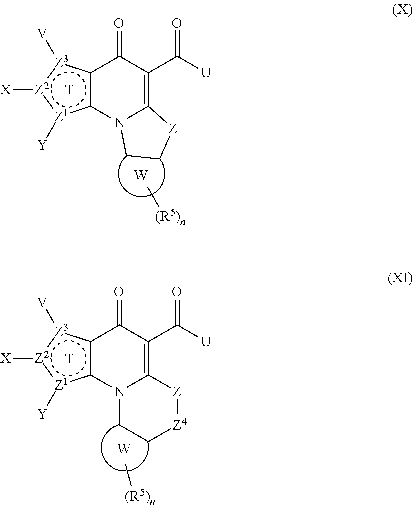

wherein U, V, W, X, Y, Z, Z¹, Z², Z³, R⁵ and n are as described above for formula (IX);

Z⁴ is CR⁶, NR², or C═O; and

Z and Z⁴ may optionally form a double bond.

In the above formula (IX), (X), and (XI), U may be NR¹OR² or NR¹NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S, wherein each ring is optionally substituted. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In other embodiments, U is NR¹NR¹—(CR¹₂)ₙ—NR³R⁴ or NR¹O—(CR¹₂)ₙ—NR³R⁴; n is 1-4; each R¹ is H or alkyl; and R³ and R⁴ in NR³R⁴ together form an optionally substituted piperidine, pyrrolidine, piperazine, morpholine, thiomorpholine, imidazole, or aminodithiazole. In some examples, U is NHNR¹—(CH₂)ₙ—NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine.

In other embodiments, U is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. In other embodiments, NR¹R² of U is an acyclic group, wherein R¹ is H or an optionally substituted C₁-C₆ alkyl such as methyl or ethyl or methoxymethyl or trifluoroethyl, and R² represents a ring such as pyridine, phenyl, pyrazine, piperidine, pyrrolidine, pyrrole, morpholine, piperazine, imidazole, imidazolidine, triazine, pyrazole, and the like.

In one embodiment, the present invention provides compounds having formula (IX), (X), or (XI), wherein:

each of V and Y if present is independently H or halogen (e.g., chloro or fluoro);

X is —NR¹R² or —CR¹R², wherein R¹ and R² together may form an optionally substituted heterocyclic, carbocyclic, aryl or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is NR¹NR¹—(CH₂)ₙ—CHR²—NR³R⁴, wherein in the —CHR²—NR³R⁴ moiety each R³ or R⁴ together with N may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In another embodiment, the present invention provides compounds having formula (IX), (X), or (XI), wherein:

V and Y if present is H or halogen (e.g., chloro or fluoro);

X if present is NR¹R², wherein, R¹ and R² together may form an optionally substituted heterocyclic or heteroaryl ring;

Z is NH or N-alkyl (e.g., N—CH₃);

W together with N and Z forms an optionally substituted 5- or 6-membered ring that is fused with an optionally substituted aryl or heteroaryl ring; and

U is —NHNR¹—(CH₂)ₙ—CHR²—NR³R⁴ or —NH—O—(CH₂)ₙ—CHR²—NR³R⁴.

In yet another aspect, the compounds of the present invention have the general formula (XII):

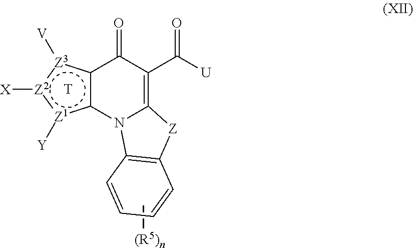

wherein U, V, X, Y, Z, Z¹, Z², Z³, R⁵ and n are as described above for formula (IX).

In yet another embodiment, the compounds of the present invention have the general formula (XIII) or (XIV):

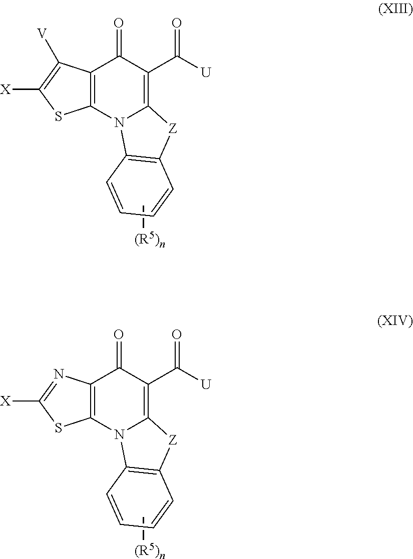

wherein U, V, X, Z, R⁵ and n are as described above for formula (IX).

In the above formula (IX)-(XIV), U may be —NHNR¹R², or —NHOR² wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S, each ring of which may be optionally substituted. For example, R² may be a C₁₋₁₀ alkyl substituted with morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodiathiazole.

In other embodiments, U is NR¹NR¹—(CR¹₂)ₙNR³R⁴; n 1-4; and R³ and R⁴ in NR³R⁴ together form an optionally substituted piperidine, pyrrolidine, piperazine, morpholine, thiomorpholine, imidazole, or aminodiathiazole. In some examples, U is NH—O—(CH₂)ₙ—NR³R⁴ wherein R³ and R⁴ together with N form an optionally substituted pyrrolidine, which may be linked to (CH₂)ₙ at any position in the pyrrolidine ring.

In other embodiments, U is NR¹NR¹R², wherein R¹ and R² cyclize together to form a ring that can be saturated or unsaturated or aromatic, and that is optionally substituted. Examples of these rings include piperidine, pyrrolidine, pyrrole, morpholine, piperazine, imidazole, imidazolidine, triazine, pyrazole, and the like. In other embodiments, NR¹R² of U is an acyclic group, wherein R¹ is H or an optionally substituted C₁-C₆ alkyl such as methyl or ethyl or methoxymethyl or trifluoroethyl, and R² represents a ring such as pyridine, phenyl, pyrazine, piperidine, pyrrolidine, pyrrole, morpholine, piperazine, imidazole, imidazolidine, triazine, pyrazole, and the like.

In the above formula (IX)-(XIV) Z may be S or NR². In some embodiments, at least one of V, X or Y when attached to C is halo.

In each of the above formula, each optionally substituted moiety may be substituted with acetyl, OR², amino, alkoxy, amido, halogen, cyano, an inorganic substituent; or a C₁₋₆ alkyl, C₂₋₆ alkenyl, C₂₋₆ alkynyl, —CONHR¹, each optionally substituted by halo, an oxo group, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or CH₂CF₃, etc.; cyano, nitro, COR², NR²COR², sulfonyl amides; NR²SOOR²; SR², SOR², COOR², CONR²₂, OCOR², OCOOR², OCONR²₂, NRCOOR², NRCONR²₂, NRC(NR)(NR²₂), NR(CO)NR²₂, and SOONR²₂, wherein each R² is as defined in formula (IX).

In one embodiment, each of Z¹, Z² and Z³ are C. In another embodiment, two of Z¹, Z² and Z³ is C, and the other is N, O or S. For example, Z² and Z³ are C, and Z¹ is S. In other examples, one of Z¹, Z² and Z³ is C and the other two are N, O, or S. For example, Z² is C, Z³ is N and Z¹ is S.

In some embodiments, each of V and X is H and the corresponding Z²-Z³ are C. In other embodiments, at least one of V and X is H and the corresponding adjacent Z²-Z³ atom is C. In yet other embodiments, one of V and X is H, and the corresponding adjacent Z²-Z³ atom is C, and the other of V and X is a bond, and the corresponding adjacent Z²-Z³ atom is N.

In certain embodiments, one of V, X and Y is a halogen (e.g., fluorine) or NR², wherein R² is a C₀₋₁₀ alkyl or C₂₋₁₀ alkenyl optionally substituted with a heteroatom, a carbocyclic ring, a heterocyclic ring, an aryl or a heteroaryl; and the corresponding adjacent Z¹-Z³ is C. In yet other examples, V, Y and X independently may be selected from alkynyls, fluorinated alkyls such as CF₃, CH₂CF₃, perfluorinated alkyls, etc.; cyano, nitro, amides, sulfonyl amides, or carbonyl compounds such as COR², and the corresponding adjacent Z¹-Z³ is C. In certain embodiments, V, X and Y are H.

In each of the above formulas (IX)-(XIV), V and X if present may independently be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. If more than one NR¹R² moiety is present in a compound within the invention, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with morpholine, thiomorpholine, imidazole, aminodithiadazole, pyrrolidine, piperazine, pyridine or piperidine. Alternatively, R¹ and R² together with N may form an optionally substituted heterocyclic ring containing one or more N, O or S. For example, R¹ and R² together with N may form piperidine, pyrrolidine, piperazine, morpholine, thiomorpholine, imidazole, or aminodithiazole.

In one embodiment, the present invention provides compounds having formula (IX), (X), or (XI), wherein:

each of V and Y are H;

X if present is H, halo or —NR¹R² or —CR¹R², wherein R⁵ is C or N and wherein in each case, R¹ and R² together may form an optionally substituted ring;

Z is S, NH or N-alkyl (e.g., N—CH₃);

W together with N and Z forms an optionally substituted 5- or 6-membered ring that is fused with an optionally substituted aryl or heteroaryl ring; and

U is —R⁵R⁶—(CH₂)ₙ—CHR²—NR³R⁴, wherein R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In certain embodiments, substituent X in a compound having a structure of Formula I, III, IV, V, VI, VII, VIII, XIII or XIV; substituent Z in a compound having a structure of Formula II; or substituent X or Y in a compound of Formula IX, X, XI or XII, can have the following structure:

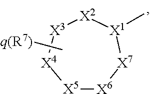

wherein X¹ is CH or N;
X², X³, X⁴, X⁵, X⁶ and X⁷ independently are NR⁶, CH₂, CHR⁷ or C(R⁷)₂, provided that: (i) zero, one or two of X², X³, X⁴, X⁵, X⁶ and X⁷ are NR⁶; (ii) when X¹ is N, both of X² and X⁷ are not NR⁶; (iii) when X¹ is N, X³ and X⁶ are not NR⁶; and (iv) when X¹ is CH and two of X², X³, X⁴, X⁵, X⁶ and X⁷ are NR⁶, the two NR⁶ are located at adjacent ring positions or are separated by two or more other ring positions;
each R⁶ is independently H, or C1-C6 alkyl; or R⁶ may be —R⁹, —R¹⁰—R⁹, —R^{H}, or —R¹⁹—N(R¹²)—R¹¹;
each R⁷ is independently halo, azido, —CN, —CF₃, —CONR¹R², —NR¹R², —SR², —OR², —R⁸, —R⁹, —R¹⁰—R⁹, —R^{H}, or —R¹⁹—N(R¹²)—R¹¹;
each R⁸ is an optionally substituted C1-C10 alkyl, C2-C10 alkenyl, C5-C10 aryl, or C6-C12 arylalkyl, or a heteroform of one of these, each of which may be optionally substituted with one or more halogens, ═O, or an optionally substituted 3-6 membered carbocyclic or heterocyclic ring;
R⁹ is an optionally substituted 4-7 membered azacyclic ring, optionally containing an additional heteroatom selected from N, O and S as a ring member;
R¹⁰ is a C1-C10 alkylene, C1-C10 heteroalkylene, C2-C10 alkenylene or C2-C10 heteroalkenylene linker, each of which may be optionally substituted with one or more substituents selected from the group consisting of halogen, oxo (═O), or C1-C6 alkyl;
R¹² is an optionally substituted 3-4 membered carbocyclic ring, or a C₁-C₆ alkyl group substituted with from 1 to 4 fluorine atoms;
R¹² is H or C1-C6 alkyl; and
q is 0, 1, 2, 3, 4, or 5.
   In certain related embodiments, X¹ is CH and two of X², X³, X⁴, X⁵, X⁶ and X⁷ are NR⁶. In some embodiments, X¹ is CH and one of X², X³, X⁴, X⁵, X⁶ and X⁷ are NR⁶. Sometimes X¹ is CH and two of X², X³, X⁴, X⁵, X⁶ and X⁷ are NR⁶. In some embodiments X¹ is CH and one of X², X³, X⁴, X⁵, X⁶ and X⁷ are NR⁶. In certain embodiments X¹ is CH and none of X², X³, X⁴, X⁵, X⁶ and X⁷ are NR⁶. Sometimes X¹ is N and none of X², X³, X⁴, X⁵, X⁶ and X⁷ are NR⁶. In certain embodiments X¹ is N and one of X⁴ or X⁵ is NR⁶, and in some embodiments X¹ is CH and none of X², X³, X⁴, X⁵, X⁶ and X⁷ are NR⁶. In some embodiments, X¹ is N and none of X², X³, X⁴, X⁵, X⁶ and X⁷ are NR⁶. In certain embodiments, X¹ is N and one of X⁴ or X⁵ is NR⁶.

The present invention also provides pharmaceutical compositions comprising at least one compound having any of the above structures, admixed with a pharmaceutically acceptable excipient. It includes individual isomers of compounds having one or more chiral centers or double bonds capable of forming stable isomers, and it includes mixtures of isomers. It also includes pharmaceutically acceptable salts of each such compound and pharmaceutical compositions of such salts.

Furthermore, the present invention provides methods for ameliorating a cell proliferative disorder, comprising administering to a subject in need thereof an effective amount of a compound having formula 1 or a pharmaceutical composition thereof, thereby ameliorating said cell-proliferative disorder. In one example, the cell proliferative disorder is cancer. In another example, cell proliferation is reduced, or cell death is induced. The subject may be human or animal.

The present invention also provides methods for reducing cell proliferation or inducing cell death, comprising contacting a system with an effective amount of a compound having formula 1 or a pharmaceutical composition thereof, thereby reducing cell proliferation or inducing cell death in said system. The system may be a cell or tissue.

The present invention further provides methods for reducing microbial titers, comprising contacting a system with an effective amount of a compound having formula 1 or a pharmaceutical composition thereof, thereby reducing microbial titers. The system may be a cell or tissue. In one example, the microbial titers are viral, bacterial or fungal titers.

Further, the present invention provides methods for ameliorating a microbial infection, comprising administering to a subject in need thereof an effective amount of a compound having formula 1 or a pharmaceutical composition thereof, thereby ameliorating said microbial infection. The subject may be human or animal. In one example, the microbial infection is viral, bacterial or fungal.

### DESCRIPTION OF THE INVENTION

The present invention relates to aroyl hydrazide and hydroxamate derivatives and related compounds that are described above, and pharmaceutically acceptable salts, esters, and prodrugs thereof. In particular embodiments, the compounds interact with regions of DNA that can form quadruplexes. The present invention also relates to methods for treating cancer, bacterial and viral infections using such compounds.

Because regions of DNA that can form quadruplexes are regulators of biological processes such as oncogene transcription and rRNA synthesis, modulators of quadruplex biological activity can be utilized as cancer therapeutics. Molecules that interact with regions of DNA that can form quadruplexes can exert a therapeutic effect on certain cell proliferative disorders and related conditions. For example, abnormally increased oncogene expression can cause cell proliferative disorders and quadruplex structures typically down-regulate oncogene expression. Examples of oncogenes include but are not limited to MYC, HIF, VEGF, ABL, TGF, PDGFA, MYB, SPARC, HUMTEL, HER, VAV, RET, H-RAS, EGF, SRC, BCL1, BCL2, and other oncogenes known to one of skill in the art. Examples of rDNA sequences that can form quadruplex structures are described in U.S. Patent Application Publication No. 20070117770, entitled “Human ribosomal DNA (rDNA) and ribosomal RNA (rRNA) nucleic acids and uses thereof,” published on May 24, 2007.

Molecules that bind to regions of DNA that can form quadruplexes can exert a biological effect according to different mechanisms, which include for example, stabilizing a native quadruplex structure, inhibiting conversion of a native quadruplex to duplex DNA by blocking strand cleavage, and stabilizing a native quadruplex structure having a quadruplex-destabilizing nucleotide substitution and other sequence specific interactions. Thus, compounds that bind to regions of DNA that can form quadruplexes described herein may be administered to cells, tissues, or organisms for the purpose of down-regulating oncogene transcription and thereby treating cell proliferative disorders. The terms “treatment” and “therapeutic effect” as used herein refer to reducing or stopping a cell proliferation rate (e.g., slowing or halting tumor growth) or reducing the number of proliferating cancer cells (e.g., removing part or all of a tumor).

Compounds described herein can be characterized in vitro or in vivo using several techniques known to the person of ordinary skill in the art. For example, a decrease or increase of RNA synthesis (e.g., rRNA synthesis) can be identified in response to contacting isolated DNA, cells or cell extracts with one or more compounds described herein. RNA synthesis products can be detected by directly observing RNA products or observing polypeptides translated by RNA products, which are methods well known in the art.

Molecules described herein can be utilized to treat many cell proliferative disorders. Cell proliferative disorders include, for example, colorectal cancers and hematopoietic neoplastic disorders (i.e., diseases involving hyperplastic/neoplastic cells of hematopoietic origin such as those arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (Vaickus, Crit. Rev. in Oncol./Hemotol. 11:267-297 (1991)). Lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease. Cell proliferative disorders also include cancers of the colorectum, breast, lung, liver, pancreas, lymph node, colon, prostate, brain, head and neck, skin, liver, kidney, and heart. Compounds that interact with regions of DNA that can form quadruplexes also can be utilized to target cancer related processes and conditions, such as increased angiogenesis, by inhibiting angiogenesis in a subject.

The present invention provides a method for reducing cell proliferation or for treating or alleviating cell proliferative disorders, comprising contacting a system having a DNA capable of forming a quadruplex with a compound of the invention. The system may be a group of cells or one or more tissues. In one embodiment, the system is a subject in need of a treatment of a cell proliferative disorder (e.g., a mammal such as a mouse, rat, monkey, or human). The present invention also provides a method for treating colorectal cancer by administering a compound that interacts with a c-MYC quadruplex forming region to a subject in need thereof, thereby reducing the colorectal cancer cell proliferation. Furthermore, the present invention provides a method for inhibiting angiogenesis and optionally treating a cancer associated with angiogenesis, comprising administering a compound that interacts with a vascular endothelial growth factor (VEGF) quadruplex forming region to a subject in need thereof, thereby reducing angiogenesis and optionally treating a cancer associated with angiogenesis.

As used herein, the term “alkyl” refers to a carbon-containing compound, and encompasses compounds containing one or more heteroatoms. The term “alkyl” also encompasses compounds substituted with one or more non-interfering substituents. Examples of non-interfering substituents include but are not limited to OR¹, amino, amido, halo, ═O, aryl, heterocyclic groups, or inorganic substituents, and other substituents that do not interfere with the activity of the compound.

As used herein, the term “carbocycle” refers to a cyclic compound containing only carbon atoms in the ring.

As used herein, the term “heterocycle” refers to a cyclic compound comprising a heteroatom, including monocyclic or bicyclic heterocycles. As used herein, the term “heteroatom” refers to any atom that is not carbon or hydrogen, such as nitrogen, oxygen or sulfur. Examples of heterocycles include but are not limited to oxirane, oxetane, pyran, tetrahydropyran, dioxane, lactones, aziridine, azetidine, pyrrolidine, piperidine, morpholine, lactams, and tetrahydrofuran.

As used herein, the term “bicyclic compound” refers to a compound having two rings which share a pair of bridgehead carbon atoms. Examples of bicyclic compounds include but are not limited to decalin, norbornane, camphor, and diazabicyclo[2.2.1]heptane.

As used herein, the terms “heteroaryl” or “heteroaromatic” refer to an aromatic heterocycle. Examples of heteroaryls include but are not limited to furan, pyrrole, pyridine, pyrimidine, imidazole, or triazole.

The terms “treat,” “treatment” and “therapeutic effect” as used herein refer to reducing or stopping a cell proliferation rate (e.g., slowing or halting tumor growth) or reducing the number of proliferating cancer cells (e.g., removing part or all of a tumor). These terms also are applicable to reducing a titre of a microorganism in a system (i.e., cell, tissue, or subject) infected with a microorganism, reducing the rate of microbial propagation, reducing the number of symptoms or an effect of a symptom associated with the microbial infection, and/or removing detectable amounts of the microbe from the system. Examples of microorganism include but are not limited to virus, bacterium and fungus.

Compounds that interact with quadruplex forming regions of DNA can also be used to reduce a microbial infection, such as a viral infection. Retroviruses offer a wealth of potential targets for G-quadruplex targeted therapeutics. G-quadruplex structures have been implicated as functional elements in at least two secondary structures formed by either viral RNA or DNA in HIV, the dimer linker structure (DLS) and the central DNA flap (CDF). Additionally, DNA aptamers which are able to adopt either inter- or intramolecular quadruplex structures are able to inhibit viral replication. In one example, DNA aptamers are able to inhibit viral replication by targeting the envelope glycoprotein (putatively). In another example, DNA aptamers inhibit viral replication by targeting the HIV-integrase respectively, suggesting the involvement of native quadruplex structures in interaction with the integrase enzyme.

Dimer linker structures, which are common to all retroviruses, serve to bind two copies of the viral genome together by a non-covalent interaction between the two 5′ ends of the two viral RNA sequences. The genomic dimer is stably associated with the gag protein in the mature virus particle. In the case of HIV, the origin of this non-covalent binding may be traced to a 98 base-pair sequence containing several runs of at least two consecutive guanines (e.g., the 3′ for the formation of RNA dimers in vitro). An observed cation (potassium) dependence for the formation and stability of the dimer in vitro, in addition to the failure of an antisense sequence to effectively dimerize, has revealed the most likely binding structure to be an intermolecular G-quadruplex.

Prior to integration into the host genome, reverse transcribed viral DNA forms a pre-integration complex (PIC) with at least two major viral proteins, integrase and reverse transcriptase, which is subsequently transported into the nucleus. The Central DNA Flap (CDF) refers to 99-base length single-stranded tail of the +strand, occurring near the center of the viral duplex DNA, which is known to a play a role in the nuclear import of the PIC. Oligonucleotide mimics of the CDF have been shown to form intermolecular G-quadruplex structures in cell-free systems.

Thus, compounds that recognize quadruplex forming regions can be used to stabilize the dimer linker structure and thus prevent de-coupling of the two RNA strands. Also, by binding to the quadruplex structure formed by the CDF, protein recognition and/or binding events for nuclear transport of the PIC may be disrupted. In either case, a substantial advantage can exist over other anti-viral therapeutics. Current Highly Active Anti-Retroviral Therapeutic (HAART) regimes rely on the use of combinations of drugs targeted towards the HIV protease and HIV integrase. The requirement for multi-drug regimes is to minimize the emergence of resistance, which will usually develop rapidly when agents are used in isolation. The source of such rapid resistance is the infidelity of the reverse transcriptase enzyme which makes a mutation approximately once in every 10,000 base pairs. An advantage of targeting viral quadruplex structures over protein targets, is that the development of resistance is slow or is impossible. A point mutation of the target quadruplex can compromise the integrity of the quadruplex structure and lead to a non-functional copy of the virus. A single therapeutic agent based on this concept may replace the multiple drug regimes currently employed, with the concomitant benefits of reduced costs and the elimination of harmful drug/drug interactions.

The present invention provides a method for reducing a microbial titer in a system, comprising contacting a system having a native DNA quadruplex forming region with a compound of the invention. The system may be one or more cells or tissues. Examples of microbial titers include but are not limited to viral, bacterial or fungal titers. In a particular embodiment, the system is a subject in need of a treatment for a viral infection (e.g., a mammal such as a mouse, rat, monkey, or human). Examples of viral infections include infections by a hepatitis virus (e.g., hepatitis B or C), human immunodeficiency virus (HIV), rhinovirus, herpes-zoster virus (VZV), herpes simplex virus (e.g., HSV-1 or HSV-2), cytomegalovirus (CMV), vaccinia virus, influenza virus, encephalitis virus, hantavirus, arbovirus, West Nile virus, human papilloma virus (HPV), Epstein-Barr virus, and respiratory syncytial virus. The present invention also provides a method for treating HIV infection by administering a compound of the invention to a subject in need thereof, thereby reducing the HIV infection.

Identifying Compounds that can Bind to Quadruplex Forming Regions of DNA

Compounds described herein are identified as compounds that can bind to quadruplex forming regions of DNA where a biological activity of this region, often expressed as a “signal,” produced in a system containing the compound is different than the signal produced in a system not containing the compound. While background signals may be assessed each time a new molecule is probed by the assay, detecting the background signal is not required each time a new molecule is assayed.

Examples of quadruplex forming regions in rDNA that can be targeted by compounds of the invention are described in U.S. Patent Application Publication No. 20070117770, entitled “Human ribosomal DNA (rDNA) and ribosomal RNA (rRNA) nucleic acids and uses thereof,” published on May 24, 2007. Examples of oncogene quadruplex forming nucleic acid sequences are set forth in the following Table A:

TABLE A

| | SEQ | |
|---|---|---|
| SEQUENCE | ID NO | ORIGIN |
| TG₄AG₃TG₄AG₃TG₄AAGG | 1 | CMYC |
| GGGGGGGGGGGGGCGGGGGCGGGGGCGGGGGAG | 2 | PDGFA |
| GGGC | | |
| G₈ACGCG₃AGCTG₅AG₃CTTG₄CCAG₃CG₄CGC | 3 | PDGFB/c- |
| TTAG₅ | | sis |
| AGGAAGGGGAGGGCCGGGGGGAGGTGGC | 4 | CABL |
| AGGGGCGGGGCGGGGCGGGGGC | 5 | RET |
| GGGAGGAAGGGGGCGGGAGCGGGGC | 6 | BCL-2 |
| GGGGGGCGGGGGCGGGCGCAGGGGGAGGGGGC | 7 | Cyclin |
| | | D1/BCL-1 |
| CGGGGCGGGGCGGGGGCGGGGGC | 8 | H-RAS |
| AGAGGAGGAGGAGGTCACGGAGGAGGAGGAGAA | 9 | CMYB |
| GGAGGAGGAGGAA | | |
| (GGA)₄ | 10 | VAV |
| AGAGAAGAGGGGAGGAGGAGGAGGAGAGGAGGA | 11 | HMGA2 |
| GGCGC | | |
| GGAGGGGGAGGGG | 12 | CPIM |
| AGGAGAAGGAGGAGGTGGAGGAGGAGG | 13 | HER2/neu |
| AGGAGGAGGAGAATGCGAGGAGGAGGGAGGAGA | 14 | EGFR |
| GGGGCGGGCCGGGGGCGGGGTCCCGGCGGGGCG | 15 | VEGF |
| GAG | | |
| CGGGAGGAGGAGGAAGGAGGAAGCGCG | 16 | CSRC |

In addition to determining whether a test molecule or test nucleic acid gives rise to a different signal, the affinity of the interaction between the nucleic acid and the compound may be quantified. IC₅₀, K_{d}, or Kᵢ threshold values may be compared to the measured IC₅₀ or K_{d} values for each interaction, and thereby identify a test molecule as a quadruplex interacting molecule or a test nucleic acid as a quadruplex forming nucleic acid. For example, IC₅₀ or K_{d} threshold values of 10 μM or less, 1 μM or less, and 100 nM or less are often utilized. In another example, threshold values of 10 nM or less, 1 nM or less, 100 pM or less, and 10 pM or less may be utilized to identify quadruplex interacting molecules and quadruplex forming nucleic acids.

Many assays are available for identifying compounds that have affinity for quadruplex forming regions of DNA. In some of these assays, the biological activity is the quadruplex nucleic acid binding to a compound and binding is measured as a signal. In other assays, the biological activity is a polymerase arresting function of a quadruplex and the degree of arrest is measured as a decrease in a signal. In certain assays, the biological activity is transcription and transcription levels can be quantified as a signal. In another assay, the biological activity is cell death and the number of cells undergoing cell death is quantified. Another assay monitors proliferation rates of cancer cells. Examples of assays are fluorescence binding assays, gel mobility shift assays (see, e.g., Jin & Pike, Mol. Endocrinol. (1996) 10:196-205), polymerase arrest assays, transcription reporter assays, cancer cell proliferation assays, and apoptosis assays (see, e.g., Amersham Biosciences (Piscataway, N.J.)), and embodiments of such assays are described hereafter. Also, topoisomerase assays can be utilized to determine whether the quadruplex interacting molecules have a topoisomerase pathway activity (see, e.g., TopoGEN, Inc. (Columbus, Ohio)).

Gel Electrophoretic Mobility Shift Assay (EMSA)

An EMSA is useful for determining whether a nucleic acid forms a quadruplex and whether a nucleotide sequence is quadruplex-destabilizing. EMSA is conducted as described previously (Jin & Pike, Mol. Endocrinol. 10: 196-205 (1996)) with minor modifications. Generally, synthetic single-stranded oligonucleotides are labeled in the 5′-terminus with T4-kinase in the presence of [γ-³²P] ATP (1,000 mCi/mmol, Amersham Life Science) and purified through a sephadex column ³²P-labeled oligonucleotides (30,000 cpm) are then incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25×Tris borate-EDTA buffer (0.25×TBE, 1×TBE is 89 mM Tris-borate, pH 8.0, 1 mM EDTA). The gel is dried and each band is quantified using a phosphoimager.

DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphoimager.

Polymerase Arrest Assay

An arrest assay includes a template nucleic acid, which may comprise a quadruplex forming sequence, and a primer nucleic acid which hybridizes to the template nucleic acid 5′ of the quadruplex-forming sequence. The primer is extended by a polymerase (e.g., Taq polymerase), which advances from the primer along the template nucleic acid. In this assay, a quadruplex structure can block or arrest the advance of the enzyme, leading to shorter transcription fragments. Also, the arrest assay may be conducted at a variety of temperatures, including 45° C. and 60° C., and at a variety of ion concentrations.

An example of the Taq polymerase stop assay is described in Han, et al., Nucl. Acids Res. (1999) 27:537-542, which is a modification of that used by Weitzmann, et al., J. Biol. Chem. (1996) 271:20958-20964. Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl, and 10 mg bromophenol blue), the reactions are separated on a preparative gel (12%) and visualized on a phosphoimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA, where “INSERT” refers to a nucleic acid sequence comprising a quadruplex forming sequence (See e.g., Table A). Bands on the gel that exhibit slower mobility are indicative of quadruplex formation.

High Throughput Polymerase Arrest Assay

A high throughput polymerase arrest assay has been developed. The assay comprises contacting a template nucleic acid, often DNA, with a primer, which also is often DNA; contacting the primer/template complex with a compound described herein (also referred to as a “test compound”); contacting the primer/template complex with a polymerase; and separating reaction products. The assay often includes the step of denaturing the primer/template complex mixture and then renaturing the complex, which often is carried out before a test molecule is added to the system. Multiple assays often are carried out using varying concentrations of a test compound, such that an IC₅₀ value can be obtained, for example. The reaction products often include extended primers of different lengths. Where a test compound does not significantly interact with a quadruplex structure in the template, the primer often is extended to the end of the template.

Where a test compound significantly interacts with a quadruplex structure in the template, the primer often is extended only to the quadruplex structure in the template and no further. Thus, the reaction mixture often includes at least two reaction products when a test compound interacts with a quadruplex structure in the template, one having a completely extended primer and one having an incompletely extended primer, and these two reaction products are separated. The products may be separated using any convenient separation method, such as mass spectrometry and in one embodiment, capillary electrophoresis.

The reaction products often are identified by detecting a detectable label linked to the primer. The detectable label may be non-covalently linked to the 5′ end of the primer (e.g., a biotin molecule covalently linked to the 5′ end of the primer which is non-covalently linked to an avidin molecule joined to a detectable label). The detectable label may be joined to the primer at any stage of the assay, sometimes before the primer is added to the system, after the primer is extended, or after the products are separated. The detectable label often is covalently linked to the primer using a procedure selected based upon the nature of the chemical groups in the detectable label.

Many methods for covalently linking detectable labels to nucleic acids are available, such as chemically coupling an allylamine-derivatized nucleotide to a succinimidyl-ester derivative of a detectable label, and then generating a primer using the labeled nucleotide. (See, e.g., Nature Biotech (2000) 18:345-348 and http address info.med.yale.edu/genetics/ward/tavi/n_coupling.html). A spacer (often between 5-16 carbon atoms long) sometimes is incorporated between the detectable label and the nucleotide. Any convenient detectable label may be utilized, including but not limited to a radioactive isotope (e.g., ¹²⁵I, ¹³¹I, ³⁵S, ³²P, ¹⁴C or ³H); a light scattering label (e.g., a spherical gold or silver label; Genicon Sciences Corporation, San Diego, Calif. and U.S. Pat. No. 6,214,560); an enzymic or protein label (e.g., GFP or peroxidase); or another chromogenic label or dye sometimes is utilized. Often, a fluorescent label is utilized (e.g., amino-methyl coumarin (AMCA); diethyl aminomethyl coumarin (DEAC); cascade blue (CB); fluorescein isothiocyanate (FITC); Oregon green (OG); Alexa 488 (A488); rhodamine green (RGr); lanthanide chelate (e.g., europium), carboxy-rhodamine 6G (R6G); tetramethyl rhodamine (TAMRA); Texas Red (TxR); Cy3; Cy3.5; Cy5, Cy5.5 and carboxynaphtofluorescein (CNF), digoxigenin (DIG); and 2,4-dinitrophenyl (DNP)). Other fluorophores and attendant excitation and emission wavelengths are described in Anantha, et al., Biochemistry (1998) 37:2709-2714 and Qu & Chaires, Methods Enzymol (2000) 321:353-369).

In an embodiment, a primer oligonucleotide covalently linked to a fluorescent label is contacted with template DNA. The resulting complex is contacted with a test molecule and then contacted with a polymerase capable of extending the primer. The reaction products then are separated and detected by capillary electrophoresis. A longer primer sequence was used for practicing this embodiment as compared to embodiments where the primer includes no covalently-linked fluorophore or where capillary electrophoresis is not utilized for separation. Deoxynucleotides are added at any stage of the assay before the separation, often when the primer is contacted with the template DNA. The template DNA/primer complex often is denatured (e.g., by increasing the temperature of the system) and then renatured (e.g., by cooling the system) before a test compound is added).

The following is a specific example of the assay embodiment. A 5′-fluorescent-labeled (FAM) primer (P45, 15 nM) was mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). The FAM-P45 primer (5′-6FAM-AGTCTGAC TGACTGTACGTAGCTAATACGACTCACTATAGCAATT-3′) and the template DNA (5′-TCCAACTATGTATACTGGGGAGGGTGGGGAGGGTGGGGAAGGTTAGCGACACG CAATTGCTATAGTGAGTCGTATTAGCTACGTACAGTCAGTCAGACT-3′) were synthesized and HPLC purified by Applied Biosystems. The mixture was denatured at 95° C. for 5 minutes and, after cooling down to room temperature, was incubated at 37° C. for 15 minutes.

After cooling down to room temperature, 1 mM KCl₂ and the test compound (various concentrations) were added and the mixture incubated for 15 minutes at room temperature. The primer extension was performed by adding 10 mM KCl and Taq DNA Polymerase (2.5 U/reaction, Promega) and incubating at 70° C. for 30 minutes. The reaction was stopped by adding 1 μl of the reaction mixture to 10 μl Hi-Di Formamide mixed and 0.25 μl LIZ120 size standard. Hi-Di Formamide and LIZ120 size standard were purchased from Applied Biosystems. The partially extended quadruplex arrest product was between 61 or 62 bases long and the full-length extended product was 99 bases long. The products were separated and analyzed using capillary electrophoresis. Capillary electrophoresis was performed using an ABI PRISM 3100-Avant Genetic Analyzer. The assay was performed using compounds described above and results are shown in Table 1. μM concentrations reported in Table 1 are concentrations at which 50% of the DNA was arrested in the assay (i.e., the ratio of shorter partially extended DNA (arrested DNA) to full-length extended DNA is 1:1).

Transcription Reporter Assay

In a transcription reporter assay, test quadruplex DNA is coupled to a reporter system, such that a formation or stabilization of a quadruplex structure can modulate a reporter signal. An example of such a system is a reporter expression system in which a polypeptide, such as luciferase or green fluorescent protein (GFP), is expressed by a gene operably linked to the potential quadruplex forming nucleic acid and expression of the polypeptide can be detected. As used herein, the term “operably linked” refers to a nucleotide sequence which is regulated by a sequence comprising the potential quadruplex forming nucleic acid. A sequence may be operably linked when it is on the same nucleic acid as the quadruplex DNA, or on a different nucleic acid. An exemplary luciferase reporter system is described herein.

A luciferase promoter assay described in He, et al., Science (1998) 281:1509-1512 often is utilized for the study of quadruplex formation. Specifically, a vector utilized for the assay is set forth in reference 11 of the He, et al., document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 minutes at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta, et al., JAGS (2001) 123:9612-9619). Spectra are arranged to represent the average of eight scans recorded at 100 nm/min

Fluorescence Binding Assay

An example of a fluorescence binding assay is a system that includes a quadruplex nucleic acid, a signal molecule, and a test molecule. The signal molecule generates a fluorescent signal when bound to the quadruplex nucleic acid (e.g., N-methylmesoporphyrin IX (NMM)), and the signal is altered when a test compound competes with the signal molecule for binding to the quadruplex nucleic acid. An alteration in the signal when test molecule is present as compared to when test compound is not present identifies the test compound as a quadruplex interacting compound.

50 μl of quadruplex nucleic acid or a nucleic acid not capable of forming a quadruplex is added in 96-well plate. A test compound also is added in varying concentrations. A typical assay is carried out in 100 μl of 20 mM HEPES buffer, pH 7.0, 140 mM NaCl, and 100 mM KCl. 50 μl of the signal molecule NMM then is added for a final concentration of 3 nM. NMM is obtained from Frontier Scientific Inc, Logan, Utah. Fluorescence is measured at an excitation wavelength of 420 nm and an emission wavelength of 660 nm using a FluoroStar 2000 fluorometer (BMG Labtechnologies, Durham, N.C.). Fluorescence often is plotted as a function of concentration of the test compound or quadruplex-targeted nucleic acid and maximum fluorescent signals for NMM are assessed in the absence of these molecules.

Cell Proliferation Assay

In a cancer cell proliferation assay, cell proliferation rates are assessed as a function of different concentrations of test compounds added to the cell culture medium. Any cancer cell type can be utilized in the assay. In one embodiment, colon cancer cells are cultured in vitro and test compounds are added to the culture medium at varying concentrations. A useful colon cancer cell line is co1o320, which is a colon adenocarcinoma cell line deposited with the National Institutes of Health as accession number JCRB0225. Parameters for using such cells are available at the http address cellbank.nihs.go.jp/cell/data/jcrb0225.htm.

Formulation of Compounds

As used herein, the term “pharmaceutically acceptable salts, esters and amides” includes but are not limited to carboxylate salts, amino acid addition salts, esters and amides of the compounds, as well as the zwitterionic forms thereof, which are known to those skilled in the art as suitable for use with humans and animals. (See, e.g., Gerge, S. M., et al., “Pharmaceutical Salts,” J. Pharm. Sci. (1977) 66:1-19, which is incorporated herein by reference.)

Any suitable formulation of the compounds described herein can be prepared. In cases where compounds are sufficiently basic or acidic to form stable nontoxic acid or base salts, administration of the compounds as salts may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids also are made.

A compound may be formulated as a pharmaceutical composition and administered to a mammalian host in need of such treatment. In one embodiment, the mammalian host is human. Any suitable route of administration may be used, including but not limited to oral, parenteral, intravenous, intramuscular, topical and subcutaneous routes.

In one embodiment, a compound is administered systemically (e.g., orally) in combination with a pharmaceutically acceptable vehicle such as an inert diluent or an assimilable edible carrier. They may be enclosed in hard or soft shell gelatin capsules, compressed into tablets, or incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may be varied and may conveniently be between about 2 to about 60% of the weight of a given unit dosage form. The amount of active compound in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active compound may be incorporated into sustained-release preparations and devices.

The active compound also may be administered intravenously or intraperitoneally by infusion or injection. Solutions of the active compound or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The compound is sometimes prepared as a polymatrix-containing formulation for such administration (e.g., a liposome or microsome). Liposomes are described for example in U.S. Pat. No. 5,703,055 (Felgner, et al.) and Gregoriadis, Liposome Technology vols. I to III (2nd ed. 1993).

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in liquid form. Compounds often are administered as compositions or formulations, in combination with a dermatologically acceptable carrier, which may be a solid or a liquid. Examples of useful dermatological compositions used to deliver compounds to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Compounds may be formulated with a solid carrier, which include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Generally, the concentration of the compound in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A compound composition may be prepared as a unit dosage form, which is prepared according to conventional techniques known in the pharmaceutical industry. In general terms, such techniques include bringing a compound into association with pharmaceutical carrier(s) and/or excipient(s) in liquid form or finely divided solid form, or both, and then shaping the product if required. The compound composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

A useful compound dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD₅₀ (the dose lethal to 50% of the population) and the ED₅₀ (the dose therapeutically effective in 50% of the population) of a compound. The dose ratio between a toxic and therapeutic effect is the therapeutic index and it can be expressed as the ratio ED₅₀/LD₅₀. The compound dosage often lies within a range of circulating concentrations for which the ED₅₀ is associated with little or no toxicity. The dosage may vary within this range depending upon the dosage form employed and the route of administration utilized. For any compounds used in the methods described herein, the therapeutically effective dose can be estimated initially from cell culture assays. A dose sometimes is formulated to achieve a circulating plasma concentration range covering the IC₅₀ (i.e., the concentration of the test compound which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” compound in the serum of the test subject. Such assays may utilize antibody mimics and/or “biosensors” generated by molecular imprinting techniques. The compound is used as a template, or “imprinting molecule”, to spatially organize polymerizable monomers prior to their polymerization with catalytic reagents. Subsequent removal of the imprinted molecule leaves a polymer matrix which contains a repeated “negative image” of the compound and is able to selectively rebind the molecule under biological assay conditions (see, e.g., Ansell, et al., Current Opinion in Biotechnology (1996) 7:89-94 and in Shea, Trends in Polymer Science (1994) 2:166-173). Such “imprinted” affinity matrixes are amenable to ligand-binding assays, whereby the immobilized monoclonal antibody component is replaced by an appropriately imprinted matrix (see, e.g., Vlatakis, et al., Nature (1993) 361:645-647). Through the use of isotope-labeling, “free” concentration of compound can be readily monitored and used in calculations of IC₅₀. Such “imprinted” affinity matrixes can also be designed to include fluorescent groups whose photon-emitting properties measurably change upon local and selective binding of compound. These changes can be readily assayed in real time using appropriate fiberoptic devices, in turn allowing the dose in a test subject to be quickly optimized based on its individual IC₅₀. An example of such a “biosensor” is discussed in Kriz, et al., Analytical Chemistry (1995) 67:2142-2144.

Exemplary doses include milligram or microgram amounts of the compound per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

A compound described herein also may be used in combination with another therapeutic agent. A therapeutic agent and compound described herein may be administered together, as separate pharmaceutical compositions, or admixed in a single pharmaceutical composition. When administered separately, the therapeutic agent and compound described herein may be administered at different times and with different frequencies, as long as the drugs are administered in a manner that yields a therapeutic effect enhanced over that of each drug alone. The therapeutic agent may be administered by any known route (e.g., orally, intravenously, intramuscularly, nasally, and the like) and may be administered by any conventional route. In certain embodiments, a therapeutic agent and compound described herein may be administered orally. The therapeutic agent in certain embodiments is a chemotherapeutic agent. Examples of chemotherapeutic agents include, but are not limited to, anti infective agents, antihelmintics, antiprotozoal agents, antimalarial agents, antiamebic agents, sulfonamides, antimycobacterial drugs, or antiviral chemotherapeutics. Chemotherapeutic agents may also be antineoplastic agents or cytotoxic drugs, such as alkylating agents, plant alkaloids, antimetabolites, antibiotics, and other anticellular proliferative agents. In other examples, the compounds and pharmaceutical formulations of the present invention may be administered with hepatic enzyme inducers or inhibitors.

The following examples are offered to illustrate but not to limit the invention.

### EXAMPLES

The following are exemplary procedures for synthesizing substituted certain quinobenzoxazine carboxylic acids that are suitable precursors for representative compounds of the invention. Methods for converting these carboxylic acids into the acyl hydrazides and hydroxamates of the invention are well known in the art. Additional compounds of the invention can similarly be prepared from the corresponding esters or acids, which are known in the art, for example, in U.S. Pat. No. 7,141,565; U.S. Pat. No. 7,163,948; U.S. patent application Ser. No. 11/149,007; U.S. patent application Ser. No. 11/404,947; U.S. Patent Application No. 60/801,550; and U.S. Patent Application No. 60/811,990, which are mentioned above.

### Example 1

### Preparation of Substituted Quinobenzoxazine Analogs

A general synthetic scheme for the preparation of substituted quinobenzoxazines analogs is shown in Scheme 1.

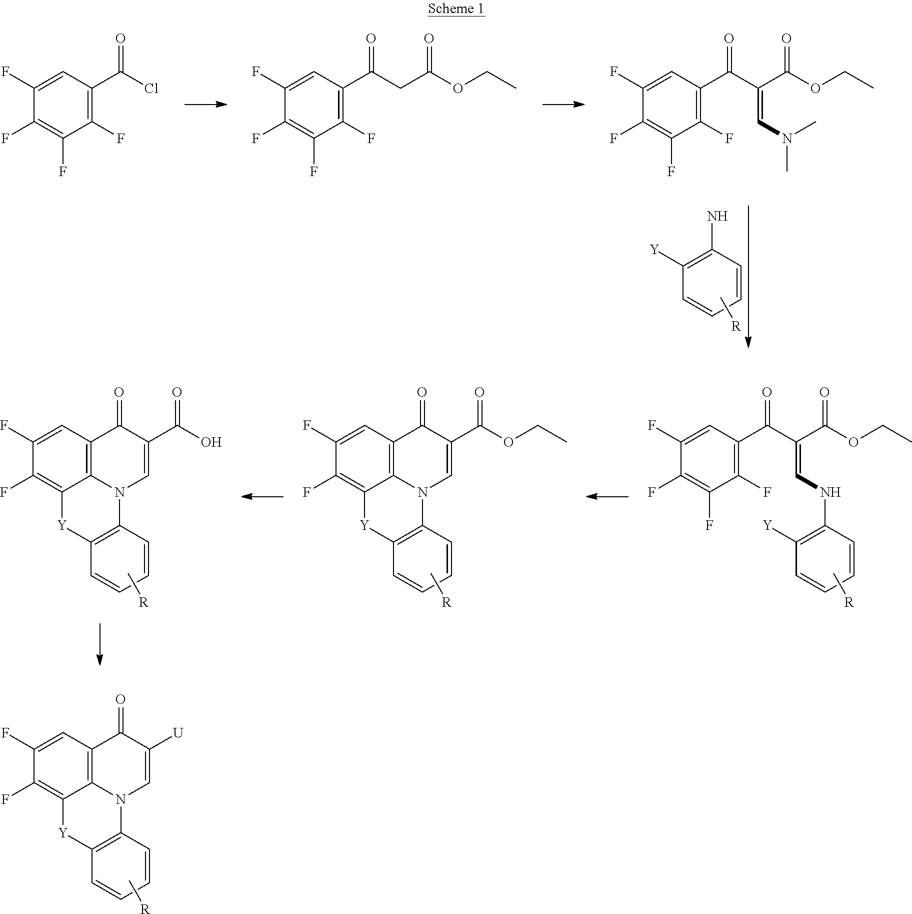

### Ethyl-(2′,3′,4′,5′-tetrafluorobenzoyl)-ethanoate

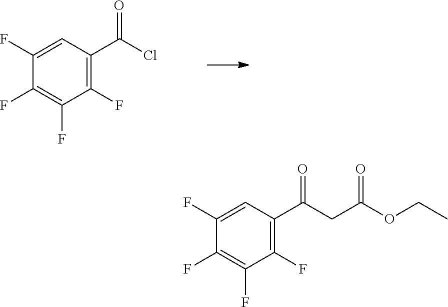

Potassium ethyl malonate (3.66 g, 21.5 mmol), MgCl₂ (2.44 g, 25.7 mmol) and TEA (2.05 g, 20.3 mmol) were mixed in acetonitrile (70 ml) at 10-15° C. for 2.5 hr. 2,3,4,5-tetrafluorobenzoyl chloride (2.00 g, 10.3 mmol) in acetonitrile (10 ml) was added at 0° C. over 15 min followed by a second addition of TEA (0.23 g, 2.3 mmol). After allowing to warm to RT, the mixture was stirred for 16 hr. After removal of volatiles in vaccuo Toluene (30 ml) was added and removed in vacuo. Following the addition of toluene (60 ml), HCl 1.5 M (40 ml) was added cautiously, ensuring the temperature did not exceed 25° C. The organic fraction was washed with HCl 1.5 M (2×25 ml) and water (2×25 ml), dried over MgSO₄ and reduced to a light orange oil in vacuo ([M+1]⁺265, 98%).

### Ethyl-2-(2′,3′,4′,5′-tetrafluorobenzoyl-)-3-(dimethylamino)-prop-2-enoate

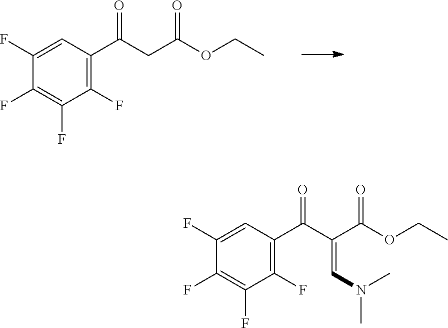

Dimethyl acetal dimethyl formamide (0.61 g, 5.1 mmol) was added dropwise to ethyl-(2′,3′,4′,5′-tetrafluorobenzoyl)-ethanoate (0.9 g, 3.41 mmol) dissolved in acetic anhydride (2 ml), under argon. After 30 min solvent was removed in vacuo to leave the product as an orange oil in a quantitative yield ([M+1⁺]320).

### Ethyl 1,2-Difluoro-4-oxo-4H-pyrido[3,2,1-kl]-8-phenyl-phenoxazine-5-carboxylate

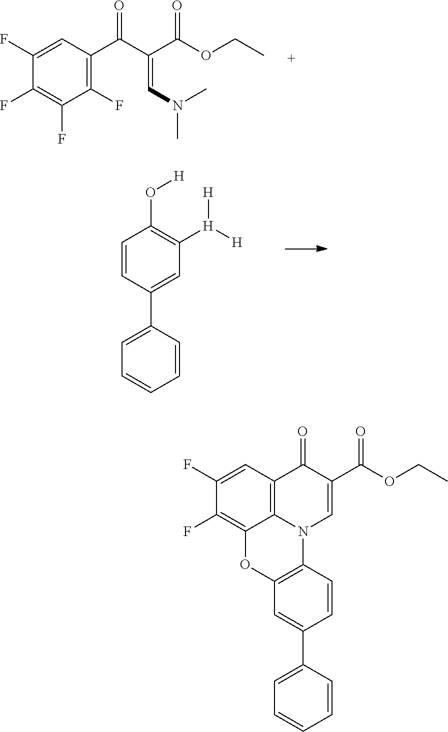

Ethyl-2-(2′,3′,4′,5′-tetrafluorobenzoyl-)-3-(dimethylamino)-prop-2-enoate (3.4 g, 8.0 mmol) and 2-amino-4-phenyl-phenol (1.5 g, 8.0 mmol) in 20 DMSO (20 ml) was stirred under vacuum at 60° C. for 30 min K2CO3 (5 g) and MeCN (20 ml) was added and the suspension was heated at 80° C. for 1 hr. After cooling to RT, the mixture was poured into a slight excess of dilute sulfuric acid and filtered. The product was recovered as a yellow-brown solid ([M+1]^{+420, 65}%).

### Example 2

### Preparation of 1,2-Difluoro-4-oxo-4H-pyrido[3,2,1-kl]-8-phenyl-phenoxazine-5-carboxylic acid

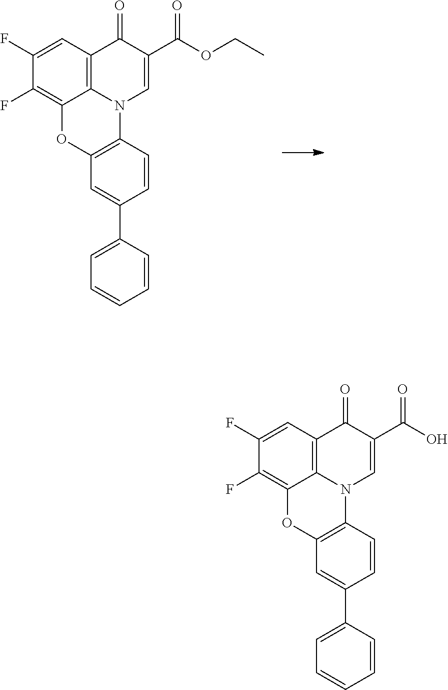

Ethyl 1,2-Difluoro-4-oxo-4H-pyrido[3,2,1-kl]-8-phenyl-phenoxazine-5-carboxylate (2.2 g, 5.3 mmol) was refluxed in a mixture of conc. HCl and acetic acid (20 ml each) for 2 hr. After cooling to room temperature cold water (40 ml) was added to the reaction mixture and the resulting precipitate filtered and washed with ether to afford the product as a yellow-brown solid 90% ([M+1]⁺392).

### Example 3

### Preparation of Ethyl-2-(2′,3′,4′,5′-tetrafluorobenzoyl-)-3-(napthyl-2″, 3″-diamino)-prop-2-enoate

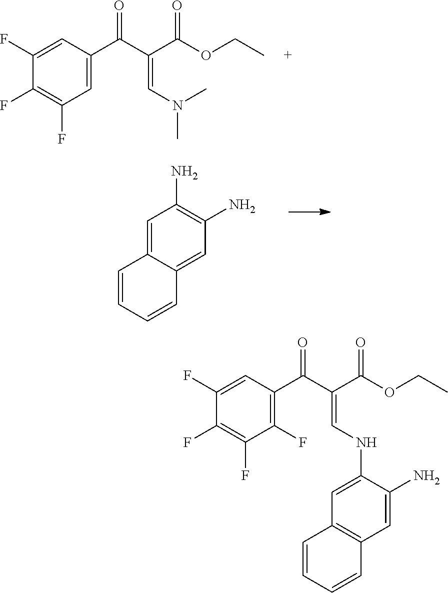

Ethyl-2-(2′,3′,4′,5′-tetrafluorobenzoyl-)-3-(dimethylamino)-prop-2-enoate (10.53 g, 33 mmol) in acetonitrile (50 ml) was added to a solution of 2,3-diaminonapthalene (5.22 g, 33 mmol) in acetonitrile (150 ml), maintained at 50° C. under argon. After 3 hours, volatiles were removed in vacuo and the residue was subjected to chromatography over silica (15% EtOAc/Hexane) to yield the product as a yellow solid ([M+1]⁺433) (55%).

### Example 4

### Preparation of Ethyl

### 1,2-Difluoro-4-oxo-4H-pyrido[3,2,1-kl]benzo[g]-phendiazine-5-carboxylate

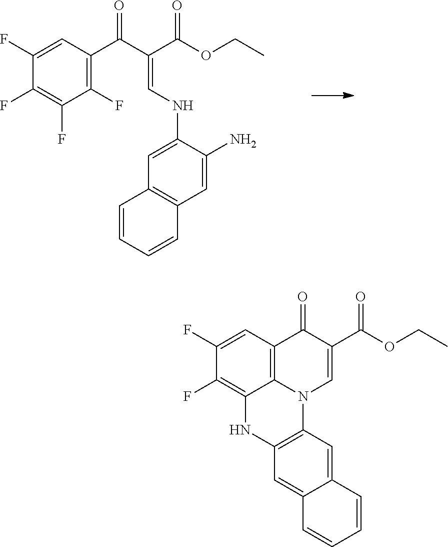

Ethyl-2-(2′,3′,4′,5′-tetrafluorobenzoyl-)-3-(napthyl-2″, 3″diamino)-prop-2-enoate (600 mg 1.4 mmol) was dissolved in a slurry of K₂CO₃ in DMF (500 ml), The mixture was stirred vigorously at 100° C. for 1 hour, then allowed to cool to RT. The K₂CO₃ was removed by filtration and the DMF removed in vacuo to leave a yellow-brown solid in quantitative yield. ([M+1]⁺393).

### Example 5

### Preparation of 1,2-Difluoro-4-oxo-4H-pyrido[3,2,1-kl]benzo[g]-phendiazine-5-carboxylic acid

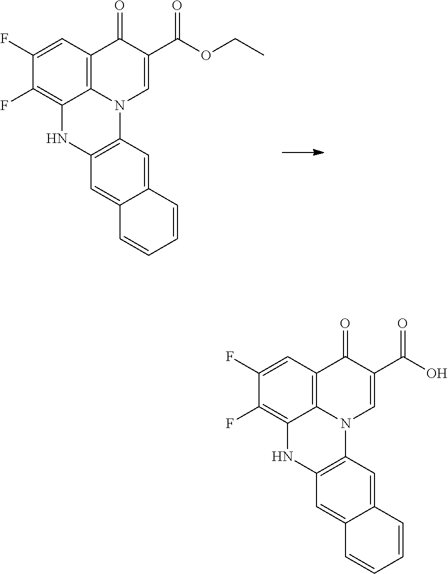

KOH solution (1N, 2.54 ml, 2.56 mmol) was added to a solution of ethyl 1,2-difluoro-4-oxo-4H-pyrido[3,2,1-kl]benzo[g]-phendiazine-5-carboxylate (500 mg, 1.28 mmol) in ethanol (400 ml), heated under reflux. After 2 hours the reaction mixture was allowed to cool to RT, then neutralized with HCl solution (1N). The product was collected by filtration as a yellow solid, 89%. ([M+1]⁺365).

### Example 6

### Preparation of Ethyl 1,2-Difluoro-4-oxo-4H-pyrido[3,2,1-kl]-phenthiazine-5-carboxylate

### Ethyl-2-(2′,3′,4′,5′-tetrafluorobenzoyl-)-3-(N-aminobenzyldisulfide)-prop-2-enoate

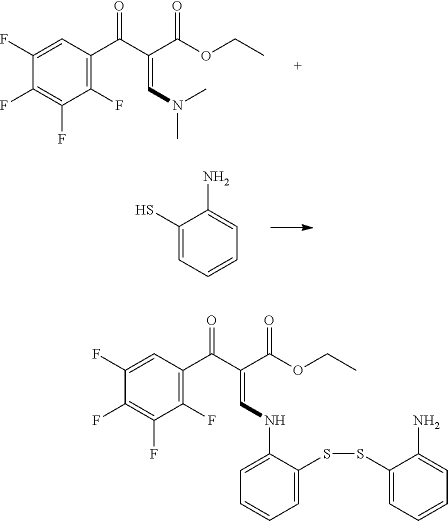

Ethyl-2-(2′,3′,4′,5′-tetrafluorobenzoyl-)-3-(dimethylamino)-prop-2-enoate (17.7 g, 55.3 mmol) in acetonitrile (10 ml) was added to a solution of 1,2-aminothiophenol dimer (5.22 g, 33 mmol) in acetonitrile (100 ml). After 3 hours, volatiles were removed in vacuo and the residue was subjected to chromatography over silica (1% MeOH/DCM) to yield the product as a yellow solid ([M+1]⁺523) (50%).

### Ethyl 1,2-Difluoro-4-oxo-4H-pyrido[3,2,1-kl]-phenthiazine-5-carboxylate

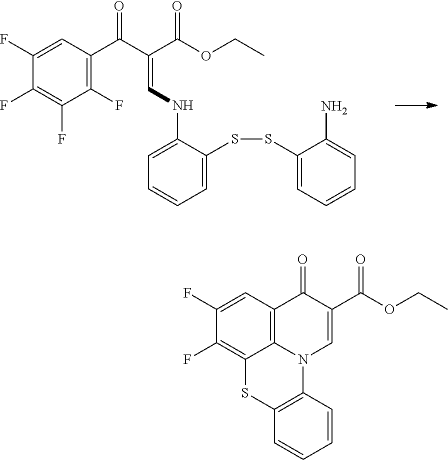

Ethyl-2-(2′,3′,4′,5′-tetrafluorobenzoyl-)-3-(N-aminobenzyldisulfide)-prop-2-enoate (2.5 g 3.2 mmol) was dissolved in DMF (120 ml) and heated under reflux for six hours. Removal of DMF in vacuo gave the product as a yellow solid 90% ([M+1]⁺360).

### Example 7

### Preparation of 1,2-Difluoro-4-oxo-4H-pyrido[3,2,1-kl]-phenthiazine-5-carboxylic acid

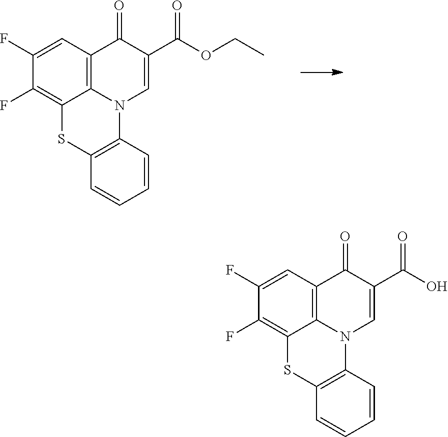

KOH solution (1N, 3.0, 3.0 mmol) was added to a solution of ethyl 1,2-difluoro-4-oxo-4H-pyrido[3,2,1-kl]-phenthiazine-5-carboxylate (1000 mg, 2.5 mmol) in ethanol (400 ml), heated under reflux. After 2 hours the reaction mixture was allowed to cool to RT, then neutralized with HCl solution (1N). The product was collected by filtration as a yellow solid, ([M+1]⁺332, 95%)

### Example 8

### Preparation of 5,6-Difluoro-9-hydroxy-3-oxo-3H-pyrido[3,2,1-kl]pyrimido[g]phenoxazine-2-carboxylic acid 7-nitroquinazoline-4,6-diol

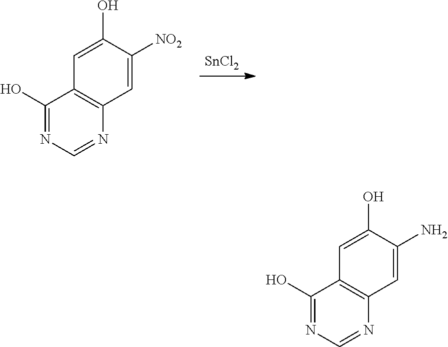

To a solution containing 20 ml of 48% aqueous HBr and 20 ml of AcOH was added 6-methoxy-7-nitro-3,4-dihydroquinazolin-4-one (1.4 g, 6.3 mmol) and the mixture was refluxed overnight. The resulting solution was evaporated to afford the crude phenol as a residue and was used without further purification (1.2 g, 5.8 mmol) (M+1, 208).

### 7-aminoquinazoline-4,6-diol

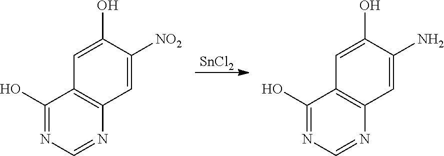

The crude product from above (1.0 g, 5.8 mmol) was diluted with 40 ml water and 3 g of Tin II chloride dihydrate was added and the reaction was stirred at room temperature. After 1 h the reaction was neutralized with K₂CO₃, and extracted with EtOAc (3×50 mL). The combined organic extracts were dried over sodium sulfate and the solvent was removed in vacuo to afford the crude amino alcohol (1.0 g, 5.6 mmol) (M+1, 178).

### Ethyl; 5,6-difluoro-9-hydroxy-3-oxo-3H-pyrido[3,2,1-kl]pyrimido[g]phenoxazine-2-carboxylate

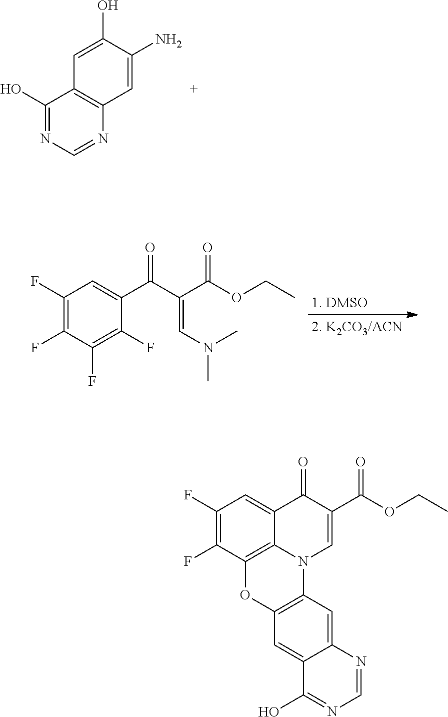

To a solution of the tetrafluoroenamine (2.2 g, 6.9 mmol) in DMSO (3 mL) was added the aminophenol (1.0 g, 5.6 mmol) and the reaction mixture was stirred under vacuum (rotary evaporator) at 60° C. for 20 minutes. The reaction mixture was allowed to cool to room temperature and was diluted with acetonitrile (200 mL) and potassium carbonate was added. The mixture was heated to reflux for 5 hours and poured into dilute HOAc/water. The solid product was collected by vacuum filtration and dried to afford the difluoroester as a tan solid (1.3 g, 3.2 mmol) (M+1, 412).

### 5,6-Difluoro-9-hydroxy-3-oxo-3H-pyrido[3,2,1-kl]pyrimido[g]phenoxazine-2-carboxylic acid

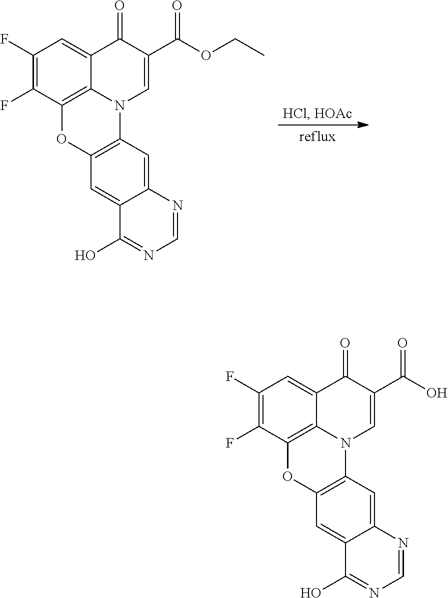

The difluoroester (1.3 g, 3.2 mmol) was dissolved in a 1:1 mixture of glacial acetic acid and 12 M HCl (20 mL) and refluxed for 30 min. The mixture was then cooled to room temperature and poured into water. The solid product was then collected by vacuum filtration and dried to afford the difluoroacid as a tan solid (0.98 g, 2.5 mmol) ([M+1]⁺392).

### Example 9

### Preparation of 2-(2-(Ethoxycarbonyl)-5,6-difluoro-3-oxo-3H-pyrido[3,2,1-kl]phenoxazin-9-yloxy)acetic acid

### Ethyl 5,6-difluoro-9-hydroxy-3-oxo-3H-pyrido[3,2,1-kl]phenoxazine-2-carboxylate

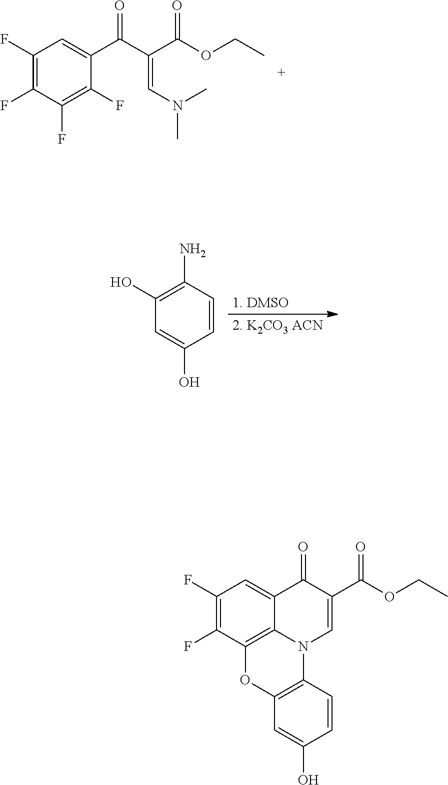

To a solution of the tetrafluoroenamine (5.8 g, 18.2 mmol), dissolved in DMSO (12 mL), was added 2,4-dihydroxyaniline hydrochloride (2.5 g, 15.5 mmol) and the mixture was heated to 60° C. under vacuum (rotary evaporator) for 20 minutes. The reaction mixture was then diluted with acetonitrile (100 mL) and potassium carbonate (3 g) was added and the mixture was refluxed overnight. The mixture was allowed to cool to room temperature and the solvent was removed in vacuo. A slight excess of 2 M HCl was added to rapidly dissolve the carbonate, and the solid precipitate was filtered and dried to afford the difluoroester as a tan solid (5.0 g, 13.9 mmol) (M+1, 360).

### 2-(2-(Ethoxycarbonyl)-5,6-difluoro-3-oxo-3H-pyrido[3,2,1-kl]phenoxazin-9-yloxy)acetic acid

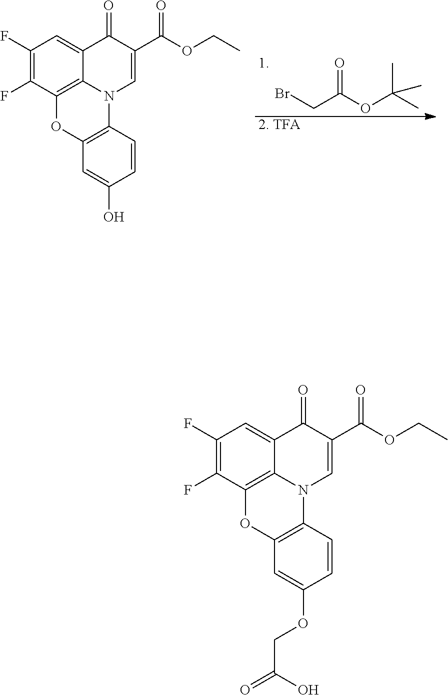

To a solution of the difluoroester (2.1 g, 5.8 mmol) and tert-butylbromoacetate (2.0 g, 10.3 mmol) in DMF (30 mL) was added potassium carbonate (2.0 g) and the mixture was heated to 60° C. for 1 hour. The reaction was allowed to cool and poured into water (500 mL) and extracted with ethyl acetate (3×100 mL), washed with brine, dried over magnesium sulfate and filtered over a pad of silica gel (30×50 mm), eluting with ethyl acetate. The solvent was removed in vacuo and the resulting material was triturated with hexanes and dried to afford the tert-butyl ester as a tan solid (2.8 g, 5.8 mmol). This material was dissolved in trifluoroacetic acid (40 mL) and stirred at room temperature for 30 minutes. The solvent was removed in vacuo to afford the acid as a tan solid (2.4 g, 5.7 mmol) (M+1, 418).

### Example 10

### Preparation of 9-(Carboxymethoxy)-5,6-difluoro-3-oxo-3H-pyrido[3,2,1-kl]phenoxazine-2-carboxylic acid

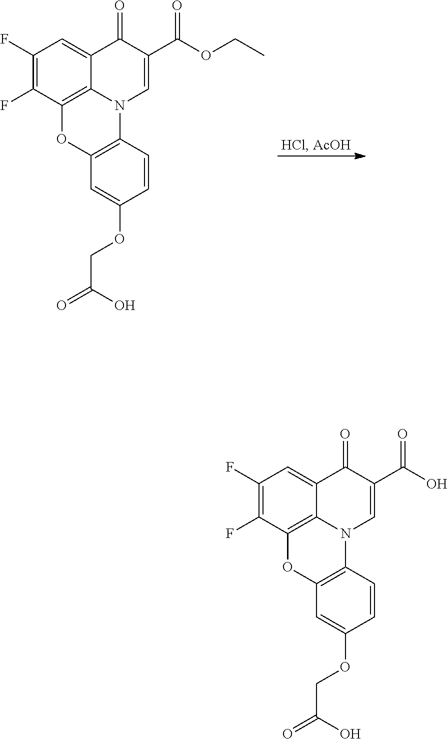

The difluoroester (2.4 g, 5.7 mmol) was dissolved in a 1:1 mixture of glacial acetic acid and 12 M HCl (40 mL) and refluxed for 1 hour. The mixture was then cooled to room temperature and poured into water. The solid product was then collected by vacuum filtration and dried to afford the difluoroacid as a tan solid (2.0 g, 5.1 mmol) (M+1, 390).

### Example 11

### Preparation of Ethyl 1,2,3-trifluoro-4-oxo-4H-pyrido[3,2,1-kl]benzo[g]-phenoxazine-5-carboxylate

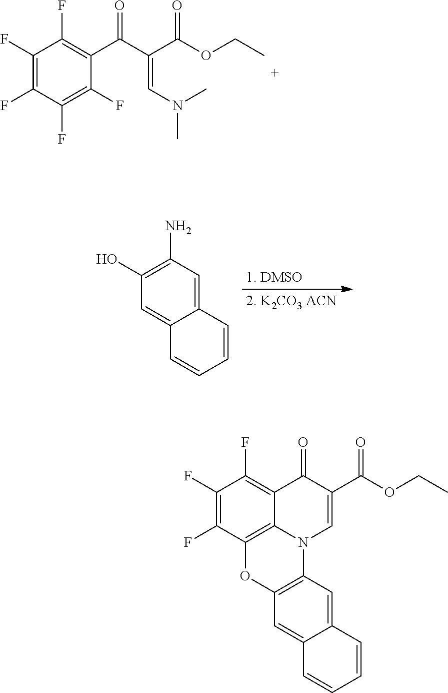

To a solution of pentafluoroenamine (8 g, 23.7 mmol), prepared by a similar procedure as for the tetrafluoroenamine dissolved in DMSO (12 mL) was added 3-amino-2-naphthol (3.5 g, 21.9 mmol) and the mixture was heated to 60° C. under vacuum (rotary evaporator) for 2 hours. The reaction mixture was then diluted with acetonitrile (200 mL) and potassium carbonate (8.0 g) was added and the mixture was refluxed overnight. The mixture was allowed to cool to room temperature and the solvent was removed in vacuo. A slight excess of 2 M HCl was added to rapidly dissolve the carbonate, and the solid precipitate was filtered and dried to afford the difluoroester as a tan solid (1.3 g, 3.2 mmol) (M+1, 412).

### Example 12

### Preparation of 1,2,3-Trifluoro-4-oxo-4H-pyrido[3,2,1-kl]benzo[g]-phenoxazine-5-carboxylic acid

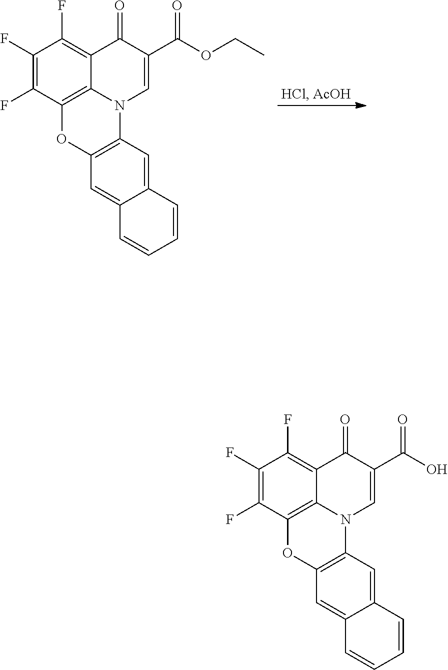

The trifluoroester (1.3 g, 3.2 mmol) was dissolved in acetic acid (5 mL) and 12 M HCl was added (5 mL) and the reaction mixture was heated to reflux for 2 hours. The mixture was then cooled to room temperature, poured into water and the solid product was collected by vacuum filtration and dried to afford the trifluoroacid as a pale solid (1.0 g, 2.6 mmol) (M+1, 384).

### Example 13

### Preparation of Ethyl 1,2-difluoro-4-oxo-4H-pyrido[3,2,1-kl]benzo[g]-phenoxazine-5-carboxylate

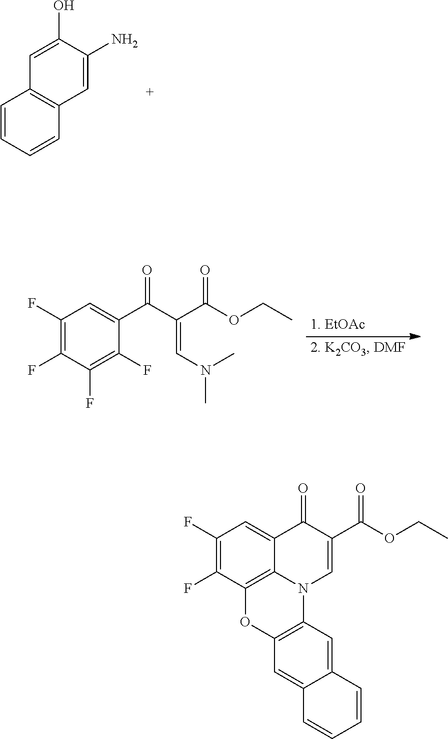

To a solution of the enamine (30 g, 94 mmol) in ethyl acetate (100 mL) was added 3-amino-2-naphthol (10 g, 63 mmol) at room temperature and the mixture was immediately placed on a rotary evaporator and the solvent was removed over 2 hours at a temperature below 0° C. (ice formed on the flask) to produce a yellow solid. To this solid was added ether (200 mL) and the slurry was filtered to afford a yellow solid. This solid was then dissolved in DMF (200 mL) and potassium carbonate was added (16.5 g, 120 mmol) and the mixture was heated to 90° C. for 1 hour. The mixture was allowed to cool to room temperature and water was added (500 mL) and the resulting solid was filtered, washed with water and dried to afford the difluoroester as a tan solid (12.2 g, 30.8 mmol) (M+1, 394).

### Example 14

### Preparation of 1,2-Difluoro-4-oxo-4H-pyrido[3,2,1-kl]benzo[g]-phenoxazine-5-carboxylic acid

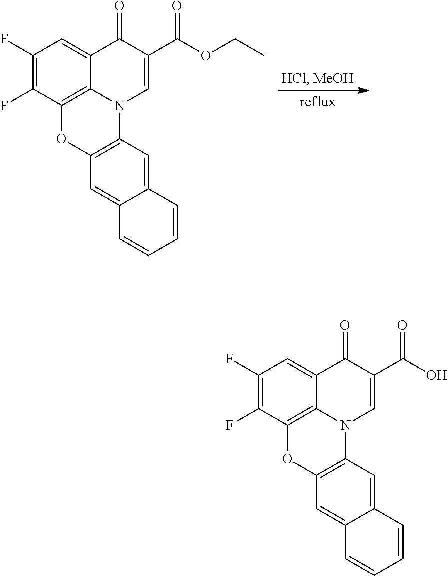

The difluoroester (5 g, 12.7 mmol) was dissolved in methanol (50 mL) and conc HCl was added (20 mL) and the mixture was refluxed for 12 hours. The mixture was allowed to cool to room temperature and the solid was collected by vacuum filtration, washing with methanol to afford the difluoroacid as a light tan powder (3.6 g, 9.9 mmol) (M+1, 366).

### Example 15

### Preparation of Ethyl 1-fluoro-4-oxo-4H-pyrido[3,2,1-kl]benzo[g]-phenoxazine-5-carboxylate

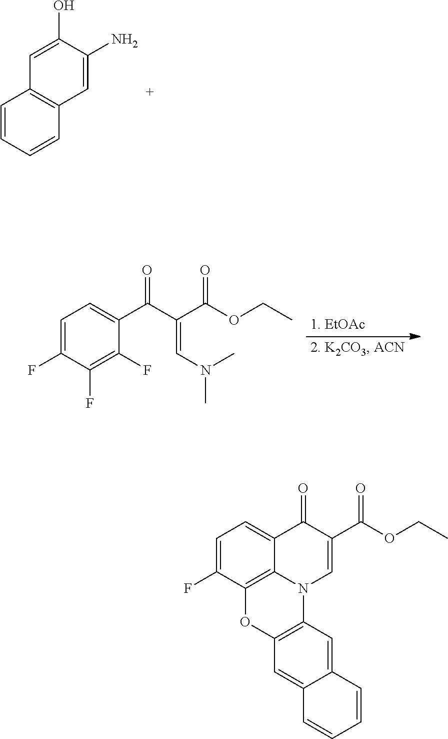

To a solution of the enamine, similarly prepared as the tetrafluoroenamine (14 g, 46.3 mmol) in ethyl acetate (100 mL) was added 3-Amino-2-naphthol (5.0 g, 31.2 mmol) at room temperature and the mixture was immediately placed on a rotary evaporator and the solvent was removed over 2 hours at a temperature below 0° C. (ice formed on the flask) to produce a yellow solid. To this solid was added methanol (200 mL) and the slurry was filtered to afford a yellow solid. This solid was then dissolved in acetonitrile (200 mL) and potassium carbonate was added (10.0 g, 72.5 mmol) and the mixture was heated to 80° C. for 1 hour. The mixture was allowed to cool to room temperature and water was added (500 mL) and the resulting solid was filtered, washed with water and dried to afford the fluoroester as a tan solid (6.0 g, 16.0 mmol) (M+1, 376).

### Example 16

### Preparation of 1-Fluoro-4-oxo-4H-pyrido[3,2,1-kl]benzo[g]-phenoxazine-5-carboxylic acid

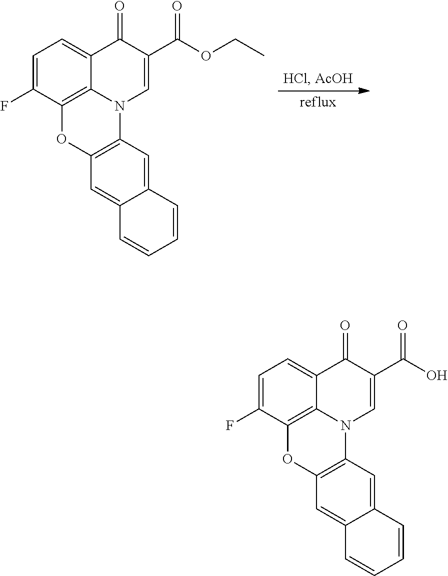

The fluoroester (6.0 g, 16.0 mmol) was dissolved in acetic acid (10 mL) and 12 M HCl was added (10 mL) and the reaction mixture was heated to reflux for 2 hours. The mixture was then cooled to room temperature, poured into water and the solid product was collected by vacuum filtration and dried to afford the fluoroacid as a pale solid (4.8 g, 13.8 mmol) (M+1, 348).

### Example 17

### Preparation of Ethyl 9-chloro-5,6-difluoro-3-oxo-3H-pyrido[3,2,1-kl]phenoxazine-2-carboxylate

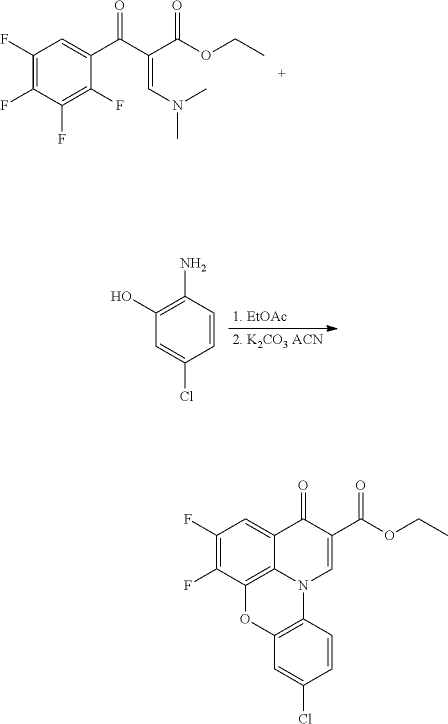

To a solution of the enamine (14.4 g, 45.3 mmol) in ethyl acetate (200 mL) was added 5-chloro-2-aminophenol (5.0 g, 34.8 mmol) and the solvent was removed in vacuo with a rotary evaporator over 2 hours without heating. Methanol was added and the resulting phenolic enamine was isolated by vacuum filtration. The resulting solid (7.0 g) was dissolved in acetonitrile and potassium carbonate was added and the resulting mixture was heated to reflux for 2 hours. The mixture was then allowed to cool to room temperature and poured into Dilute HCl. The resulting solid was collected by vacuum filtration and dried to afford the difluoroester as a pale yellow solid (5.0 g, 13.3 mmol) (M+1, 378).

### Example 18

### Preparation of Ethyl 9-chloro-5-fluoro-6-(4-methyl-1,4-diazepan-1-yl)-3-oxo-3H-pyrido[3,2,1-kl]phenoxazine-2-carboxylate

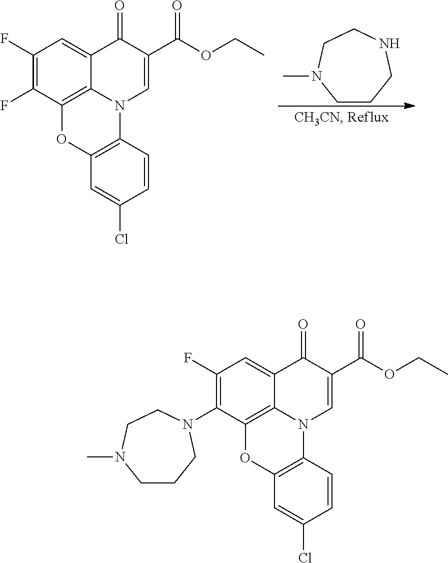

To a solution of the difluoroester (1.16 g, 3.07 mmol) dissolved in acetonitrile (15 mL) an amine was added (1.3 ml, 10.36 mmol) and the reaction mixture was heated at reflux overnight. The mixture was then cooled to room temperature and acetonitrile was removed under vacuum. The residue obtained was partitioned between water and ethyl acetate. The organic layer was isolated, dried with sodium sulfate and concentrated under vacuum. The residue obtained was passed through silica plug eluting with 1% methanol in dichloromethane.

The combined fractions were concentrated under vacuum to give the product as yellow solid (M+1, 472).

### Example 19

### Preparation of 9-Chloro-5,6-difluoro-3-oxo-3H-pyrido[3,2,1-kl]phenoxazine-2-carboxylic acid

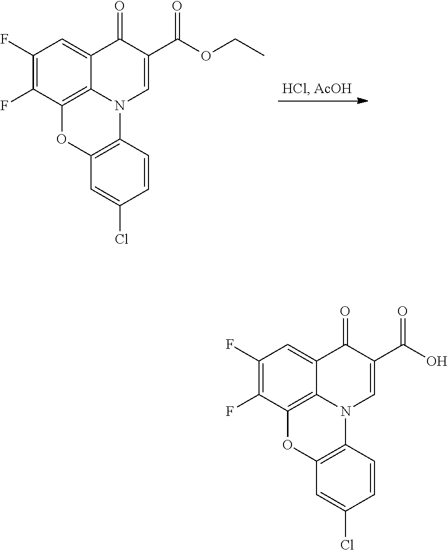

The difluoroester (5.0 g, 13.3 mmol) was dissolved in acetic acid (45 mL) and 12 M HCl was added (30 mL) and the reaction mixture was heated to reflux for 2 hours. The mixture was then cooled to room temperature, poured into water and the solid product was collected by vacuum filtration and dried to afford the difluoroacid as a pale solid (4.0 g, 10.6 mmol) (M+1, 350).

### Example 20

### Preparation of Ethyl b 10-chloro-5,6-difluoro-3-oxo-3H-pyrido[3,2,1-kl]phenoxazine-2-carboxylate

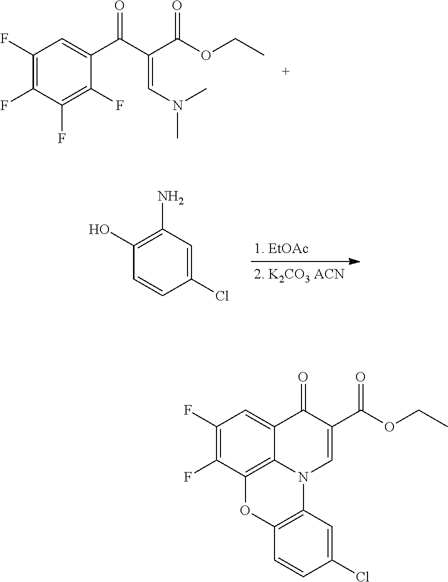

To a solution of the enamine (14.4 g, 45.3 mmol) in ethyl acetate (200 mL) was added 4-chloro-2-aminophenol (5.0 g, 34.8 mmol) and the solvent was removed in vacuo with a rotary evaporator over 2 hours without heating. Methanol was added and the resulting phenolic enamine was isolated by vacuum filtration. The resulting solid (7.5 g) was dissolved in acetonitrile and potassium carbonate was added and the resulting mixture was heated to reflux for 2 hours. The mixture was then allowed to cool to room temperature and poured into Dilute HCl. The resulting solid was collected by vacuum filtration and dried to afford the difluoroester as a pale yellow solid (5.0 g, 13.3 mmol) (M+1, 378).

### Example 21

### Preparation of 10-Chloro-5,6-difluoro-3-oxo-3H-pyrido[3,2,1-kl]phenoxazine-2-carboxylic acid

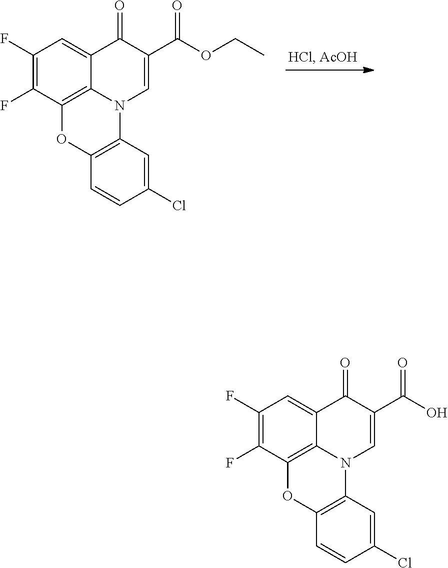

The difluoroester (2.5 g) was dissolved in acetic acid (25 mL) and 12 M HCl was added (20 mL) and the reaction mixture was heated to reflux for 2 hours. The mixture was then cooled to room temperature, poured into water and the solid product was collected by vacuum filtration and dried to afford the difluoroacid as a pale solid (2.0 g, 5.3 mmol) (M+1, 350).

### Example 22

### Preparation of Ethyl 5,6-Difluoro-3-oxo-3H-pyrido[3,2,1-kl]phenoxazine-2-carboxylate

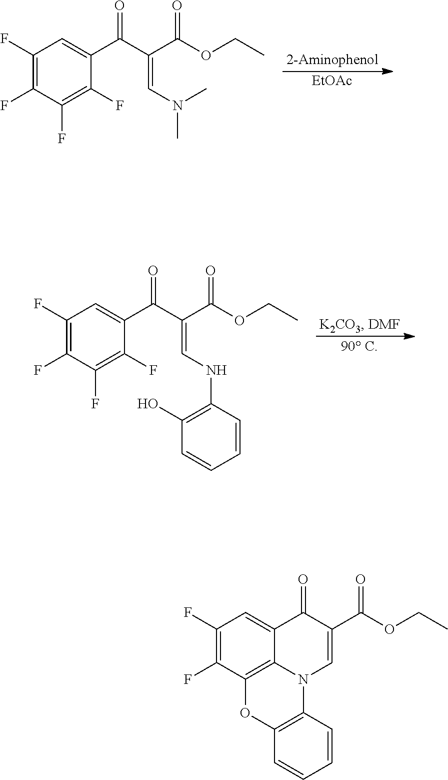

To a solution of the enamine (5.7 g, 17.9 mmol) in ethyl acetate (50 mL) was added 2-aminophenol (1.9 g, 17.43 mmol) at room temperature and the mixture was immediately placed on a rotary evaporator and the solvent was removed over 2 hours at a temperature below 0° C. (ice formed on the flask) to produce a yellow solid. To this solid was added ether (25 mL) and the slurry was filtered to afford a yellow solid. This solid was then dissolved in DMF (20 mL) and potassium carbonate was added (2.9 g, 21 mmol) and the mixture was heated to 90° C. for 1 hour. The mixture was allowed to cool to room temperature and water was added (200 mL) and the resulting solid was filtered, washed with water and dried to afford the phenoxazine as a tan solid (2.9 g, 8.45 mmol) (M+1, 344).

### Example 23

### Preparation of 5,6-Difluoro-3-oxo-3H-pyrido[3,2,1-kl]phenoxazine-2-carboxylic acid

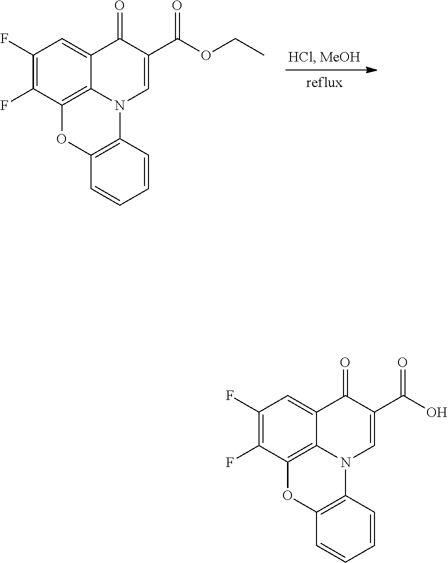

The difluoroester (5.0 g, 14 mmol) was dissolved in methanol (50 mL) and conc HCl was added (20 mL) and the mixture was refluxed for 2 hours. The mixture was allowed to cool to room temperature and the solid was collected by vacuum filtration, washing with methanol to afford the difluoroacid as a light tan powder (4.2 g, 13.3 mmol, 91%) (M+1, 316).

### Example 24

### Preparation of Ethyl 1,2-Difluoro-4-oxo-4H-pyrido[3,2,1-kl]benzo[h]-phenoxazine-5-carboxylate

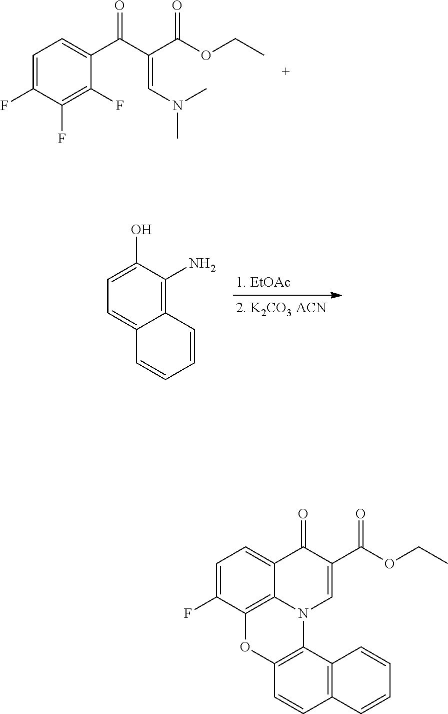

To a solution of the enamine (14.0 g, 45.3 mmol) in ethyl acetate (200 mL) was added 1-amino-2-naphthol (5.0 g, 31.3 mmol) and the solvent was removed in vacuo with a rotary evaporator over 2 hours without heating. Methanol was added and the resulting phenolic enamine was isolated by vacuum filtration. The solid was dissolved in acetonitrile and potassium carbonate (10 g) was added and the mixture was heated to reflux for 2 hours. The mixture was then allowed to cool to room temperature and poured into Dilute HCl. The resulting solid was collected by vacuum filtration and dried to afford the difluoroester as a pale yellow solid (5.0 g, 13.3 mmol) (M+1, 376).

### Example 25

### Preparation of 1,2-Difluoro-4-oxo-4H-pyrido[3,2,1-kl]benzo[h]-phenoxazine-5-carboxylic acid

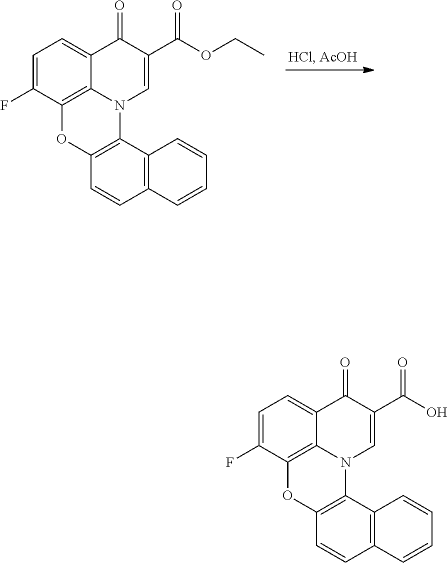

The difluoroester (5.5 g) was dissolved in acetic acid (25 mL) and 12 M HCl was added (20 mL) and the reaction mixture was heated to reflux for 2 hours. The mixture was then cooled to room temperature, poured into water and the solid product was collected by vacuum filtration and dried to afford the difluoroacid as a pale solid (5.0 g, 14.4 mmol) (M+1, 348).

### Example 26

### Preparation of Ethyl 1,2-Difluoro-4-oxo-4H-pyrido[3,2,1-kl]benzo[f]-phenoxazine-5-carboxylate

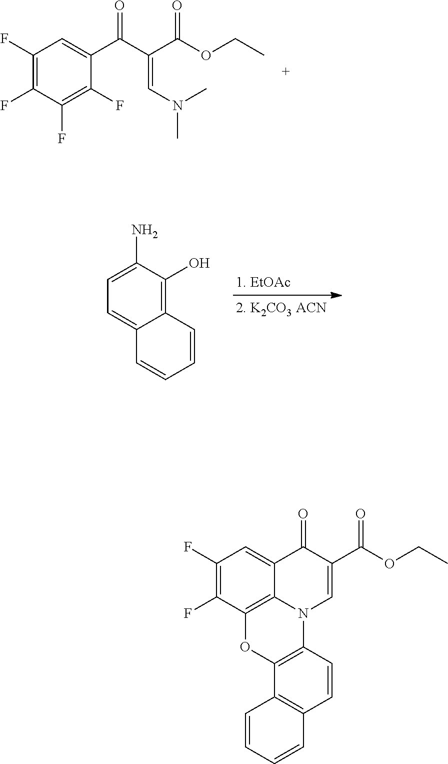

To a solution of the enamine (45 g, 141 mmol) in ethyl acetate (500 mL) was added 2-amino-1-naphthol (15.0 g, 93.8 mmol) and the solvent was removed in vacuo with a rotary evaporator over 2 hours without heating. Methanol was added and the phenolic enamine was isolated by vacuum filtration. The resulting solid was dissolved in acetonitrile (400 mL) and potassium carbonate (25 g) was added and the mixture was heated to reflux for 2.5 hours. The mixture was then allowed to cool to room temperature and poured into Dilute HCl. The resulting solid was collected by vacuum filtration and dried to afford the difluoroester as a pale yellow solid (19.69 g, 50.1 mmol) (M+1, 394).

### Example 27

### Preparation of 1,2-Difluoro-4-oxo-4H-pyrido[3,2,1-kl]benzo[f]-phenoxazine-5-carboxylic acid

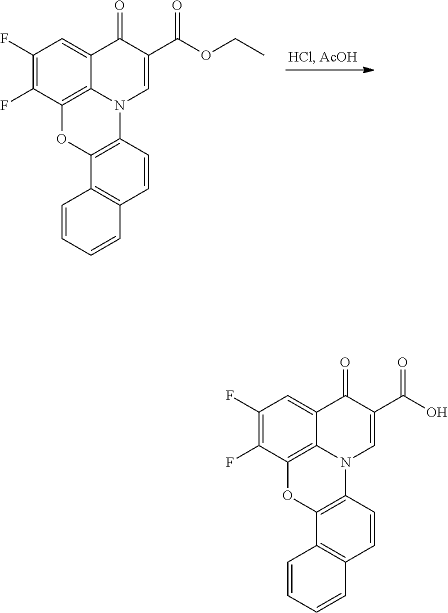

The difluoroester (15.0 g, 38.1 mmol) was dissolved in acetic acid (60 mL) and 12M HCl was added (60 mL) and the reaction mixture was heated to reflux for 2 hours. The mixture was then cooled to room temperature, poured into water and the solid product was collected by vacuum filtration and dried to afford the difluoroacid as a pale solid (11.7 g, 32 mmol) (M+1, 366).

### Example 28

### Preparation of Ethyl 1,2-Difluoro-4-oxo-4H-pyrido[3,2,1-kl]benzo[b]furan[2,3-g]-phenoxazine-5-carboxylate

### 3-Aminodibenzofuran-2-ol

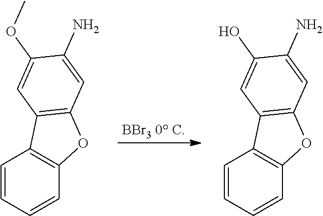

To a solution of the dibenzofuran (15 g, 70.4 mmol) dissolved in methylene chloride (500 mL) at 0° C. was added BBr₃ (200 mL, 1 M in CH₂Cl₂) via addition funnel. After the addition was complete, the mixture was allowed to come to room temperature over 1 hour and then quenched with water followed by potassium carbonate (40 g). The resulting solid was recovered by vacuum filtration and dried to afford the hydroxyl dibenzofuran as a white solid (13.2 g, 199 mmol) (M+1, 200).

### Ethyl 1,2-Difluoro-4-oxo-4H-pyrido[3,2,1-kl]benzo[b]furan[2,3-g]-phenoxazine-5-carboxylate

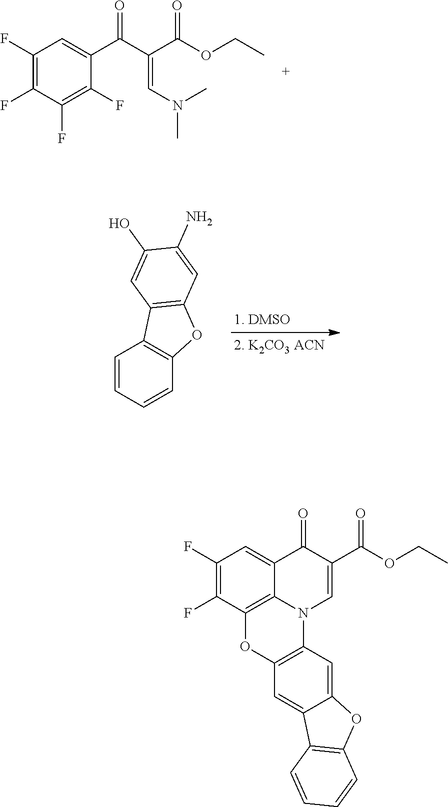

To a solution of the tetrafluoroenamine (15.0 g, 47 mmol) dissolved in DMSO (30 mL) was added the hydroxyl dibenzofuran (12.0 g, 60 mmol) and the mixture was heated to 60° C. under vacuum (rotary evaporator) for 20 minutes. The reaction mixture was then diluted with acetonitrile (200 mL) and potassium carbonate (17 g) was added and the mixture was refluxed for 2.5 hours. The mixture was allowed to cool to room temperature and the solvent was removed in vacuo. A slight excess of 2 M HCl was added to rapidly dissolve the carbonate, and the solid precipitate was filtered and dried to afford the difluoroester as a tan solid (15.0 g, 34.6 mmol) (M+1, 434).

### Example 29

### Preparation of 1,2-Difluoro-4-oxo-4H-pyrido[3,2,1-kl]benzo[b]furan[2,3-g]-phenoxazine-5-carboxylic acid

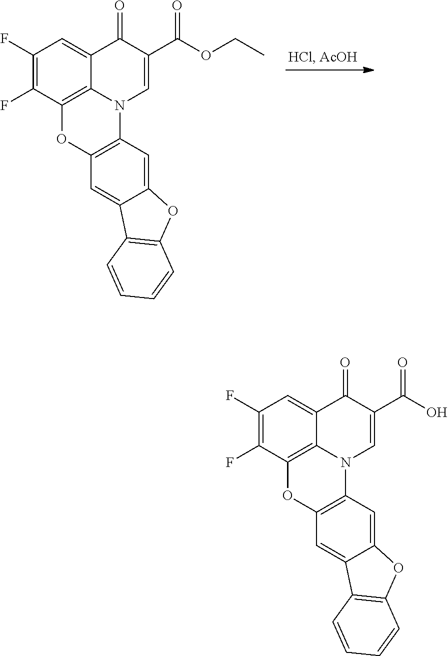

The difluoroester (15.0 g, 34.6 mmol) was dissolved in acetic acid (60 mL) and 12 M HCl was added (60 mL) and the reaction mixture was heated to reflux for 2 hours. The mixture was then cooled to room temperature, poured into water and the solid product was collected by vacuum filtration and dried to afford the difluoroacid as a pale solid (13.7 g, 34 mmol) (M+1, 406).

### Example 30

### Preparation of Ethyl 2-(ethoxycarbonyl)-5,6-difluoro-3-oxo-3H-pyrido[3,2,1-kl]phenoxazine-10-carboxylate

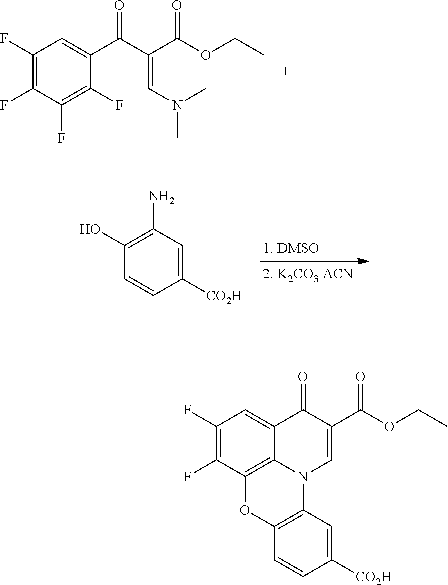

To a solution of the tetrafluoroenamine (7.0 g, 21.9 mmol) dissolved in DMSO (25 mL) was added 4-hydroxy-3-amino benzoic acid (3.0 g, 19.6 mmol) and the mixture was heated to 60° C. under vacuum (rotary evaporator) for 2 hours. The reaction mixture was then diluted with acetonitrile (200 mL) and potassium carbonate (8.0 g) was added and the mixture was refluxed overnight. The mixture was allowed to cool to room temperature and the solvent was removed in vacuo. A slight excess of 2 M HCl was added to rapidly dissolve the carbonate, and the solid precipitate was filtered and dried to afford the difluoroester as a tan solid (6.2 g, 16.0 mmol) (M+1, 388).

### Example 31

### Preparation of 2-(Ethoxycarbonyl)-5,6-difluoro-3-oxo-3H-pyrido[3,2,1-kl]phenoxazine-10-carboxylic acid

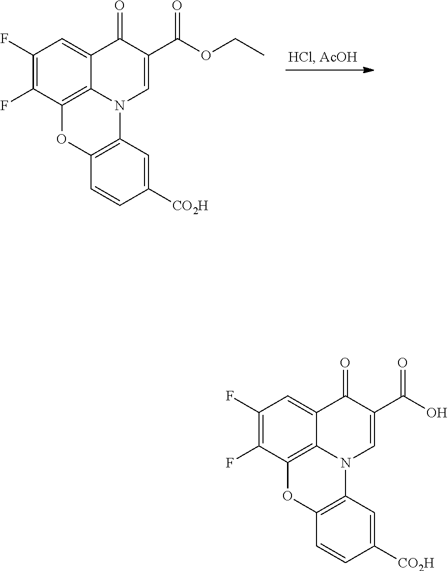

The difluoroester (6.2, 16.0 mmol g) was dissolved in acetic acid (25 mL) and 12 M HCl was added (20 mL) and the reaction mixture was heated to reflux for 2 hours. The mixture was then cooled to room temperature, poured into water and the solid product was collected by vacuum filtration and dried to afford the difluorodi-acid as a pale solid (5.3 g, 14.8 mmol) (M+1, 360).

### Example 32

### Preparation of Ethyl 5,6-difluoro-10-nitro-3-oxo-3H-pyrido[3,2,1-kl]phenoxazine-2-carboxylate

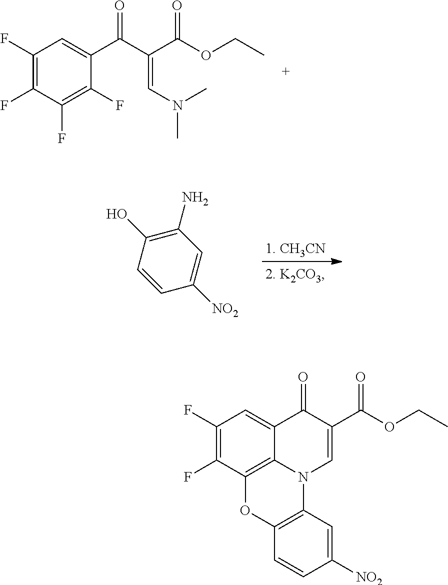

A solution of the enamine (6.0 g, 18.7 mmol) and 2-amino-4-nitrophenol (3.5 g, 23.3 mmol) in acetonitrile was heated to 80° C. for 15 minutes. Potassium carbonate was then added (8.0 g) and the mixture was heated to reflux overnight. The reaction mixture was then filtered hot and the solvent was removed in vacuo to afford the crude nitroester (5.0 g, 12.8 mmol) (M+1, 389).

### Example 33

### Preparation of 5,6-Difluoro-10-nitro-3-oxo-3H-pyrido[3,2,1-kl]phenoxazine-2-carboxylic acid

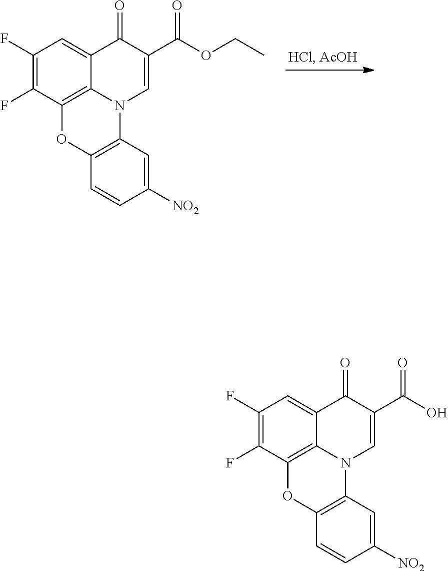

The crude difluoroester (5.0 g, 12.8 mmol) was dissolved in acetic acid (25 mL) and 12 M HCl was added (20 mL) and the reaction mixture was heated to reflux for 2 hours. The mixture was then cooled to room temperature, poured into water and the solid product was collected by vacuum filtration and dried to afford the difluoroacid as a pale solid (2.0 g, 5.5 mmol) (M+1, 361).

### Example 34

### Preparation of Ethyl 5,6-difluoro-3-oxo-10-phenyl-3H-pyrido[3,2,1-kl]phenoxazine-2-carboxylate

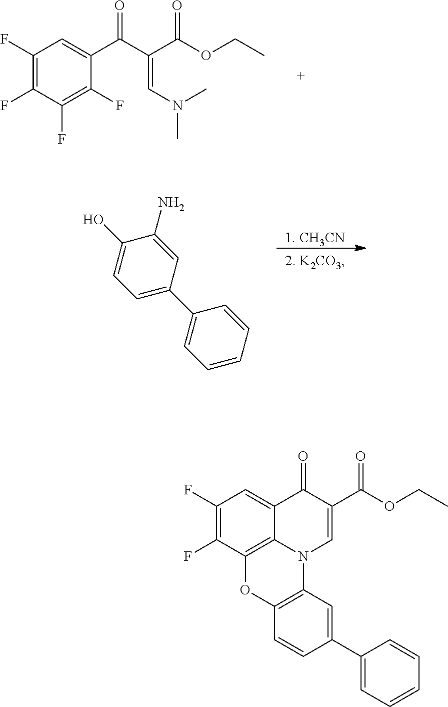

A solution of the enamine (5.4 g, 16.9 mmol) and 3-amino-4-hydroxybiphenyl (3.5 g, 18.9 mmol) in acetonitrile was heated to 80° C. for 90 minutes. Potassium carbonate was then added (8.0 g) and the mixture was heated to reflux overnight. The reaction mixture was then filtered hot and the solvent was removed in vacuo to afford the crude difluoroester (3.9 g, 9.3 mmol) (M+1, 420).

### Example 35

### Preparation of 5,6-Difluoro-3-oxo-10-phenyl-3H-pyrido[3,2,1-kl]phenoxazine-2-carboxylic acid

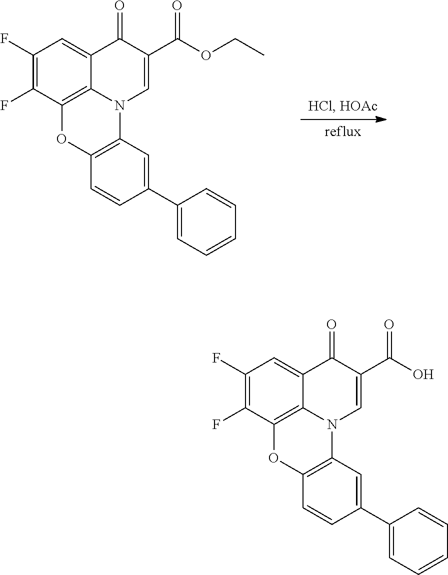

The crude difluoroester (3.6 g, 8.6 mmol) was dissolved in acetic acid (10 mL) and 12 M HCl was added (10 mL) and the reaction mixture was heated to reflux for 2 hours. The mixture was then cooled to room temperature, poured into water and the solid product was collected by vacuum filtration and dried to afford the difluoroacid as a pale solid (2.6 g, 6.6 mmol) (M+1, 392).

### Example 36

### Preparation of Ethyl 1,2-Difluoro-4-oxo-4H-11-sulfonic-acid-pyrido[3,2,1-kl]benzo[h]-phenoxazine-5-carboxylate

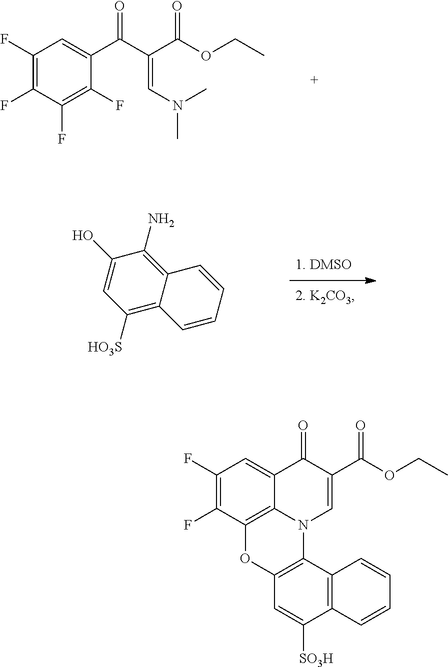

To a solution of the tetrafluoroenamine (5.4 g, 16.9 mmol) dissolved in DMSO (30 mL) was added 1-amino-2-hydroxy-4-naphthalenesulfonic acid (4.8 g, 20 mmol) and the mixture was heated to 60° C. under vacuum (rotary evaporator) for 2 hours. To the reaction mixture was added potassium carbonate (10.0 g) and the mixture was heated to 60° C. for 1 hour. The mixture was allowed to cool to room temperature and a slight excess of 2 M HCl was added to rapidly dissolve the carbonate. The aqueous layer was decanted and the remaining organic residue was dissolved in methanol (100 mL) and precipitated with ethyl acetate (200 mL) and the solid precipitate was filtered and dried to afford the sulfonic acid as a brown solid (3.1 g, 6.5 mmol) (M+1, 474).

### Example 37

### Preparation of 1,2-Difluoro-4-oxo-4H-11-sulfonic-pyrido[3,2,1-kl]benzo[h]-phenoxazine-5-carboxylic acids

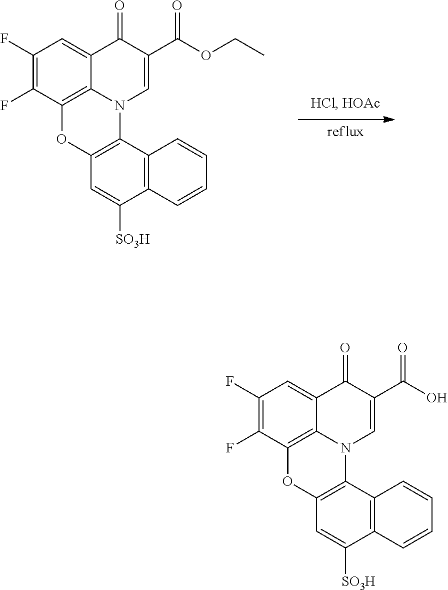

The crude difluoroester (1.5 g, 3.2 mmol) was dissolved in acetic acid (10 mL) and 12 M HCl was added (10 mL) and the reaction mixture was heated to reflux for 30 minutes. The solvent was removed in vacuo to afford the sulfonic acid as a brown solid (1.1 g, 2.5 mmol) (M+1, 446)

### Example 38

### Preparation of 2-(Ethoxycarbonyl)-5,6-difluoro-3-oxo-3H-pyrido[3,2,1-kl]phenoxazine-9-carboxylic acid

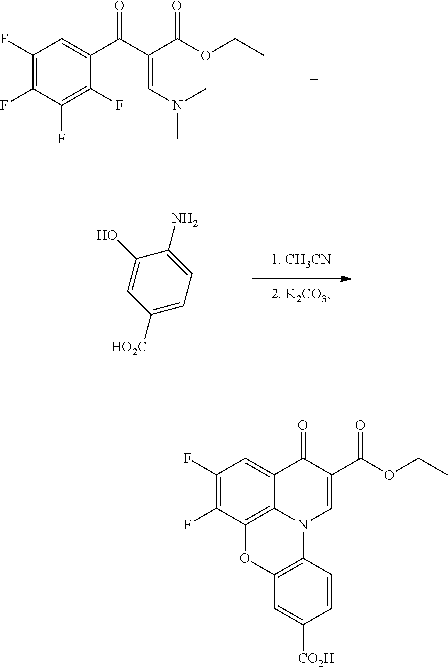

A solution of the difluoroenamine (5.2 g, 16.3 mmol) and 4-amino-3-hydroxybenzoic acid (4.0 g, 26.1 mmol) in DMSO was stirred at room temperature for 1.5 hours. Potassium carbonate (8 g) was then added and the reaction mixture was stirred under vacuum (rotary evaporator) for 1 hour. The mixture was then heated to 100° C. for 1 hour and then allowed to cool to room temperature. The reaction mixture was then poured into 1 M H₂SO₄ (500 mL) and the solids were recovered by vacuum filtration. The resulting solid was dried to afford the crude difluoroacid as a tan solid (5.0 g, 12.9 mmol) (M+1, 388).

### Example 39

### Preparation of 5,6-Difluoro-3-oxo-3H-pyrido[3,2,1-kl]phenoxazine-2,9-dicarboxylic acid

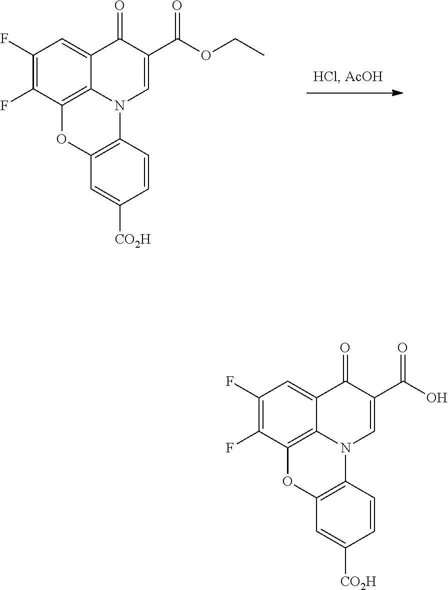

The crude difluoroester (5.0 g, 12.9 mmol) was dissolved in acetic acid (20 mL) and 12 M HCl was added (20 mL) and the reaction mixture was heated to reflux for 1 hour. The reaction was allowed to cool to room temperature and water was added. The resulting solid was collected by vacuum filtration and dried overnight to afford the di-acid as a tan solid (1.9 g, 5.3 mmol) (M+1, 360).

### Example 40

### Preparation of Ethyl 1,2-Difluoro-4-oxo-4H-pyrido[3,2,1-kl]-8-fluorenone-5-carboxylate

### 3-Nitro-2-hydroxyfluorenone

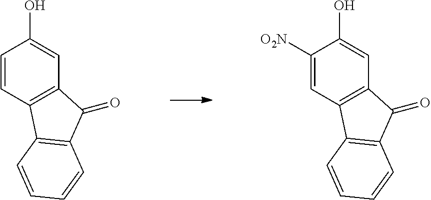

A solution of NO₂BF₄ (3.52 g, 25.5 mmol) in acetonitrile (100 ml) was added dropwise to a solution of 2-hydroxyfluorenone (5 g, 25.5 mmol) in acetonitrile (400 ml) at ambient temperature. The reaction mixture was then cooled to 0° C. and water (100 ml) was added to precipitate impurities. After filtration, water (200 ml) was added and the precipitate filtered off as a red solid (68%) (M+1, 242).

### 3-amino-2-hydroxyfluorenone

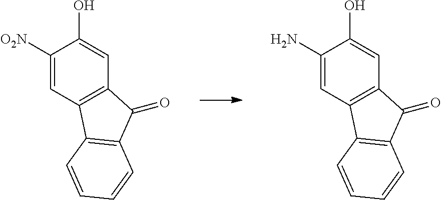

A mixture of 3-nitro-2-hydroxyfluorenone (1.6 g, 6.6 mmol) and SnCl₂ (3 g, 6.6 mmol) was refluxed in 100 ml acetic acid:conc. HCl (1:1) for 1 hour. The mixture was allowed to cool to room temperature and neutralized with ammonium hydroxide. After extracting with EtOAc (3×100 ml), combined organic fractions were dried over magnesium sulfate and evaporated to leave the product as a brown solid (65%) (M+1, 212).

### Ethyl 1,2-Difluoro-4-oxo-4H-pyrido[3,2,1-kl]-8-fluorenone-5-carboxylate

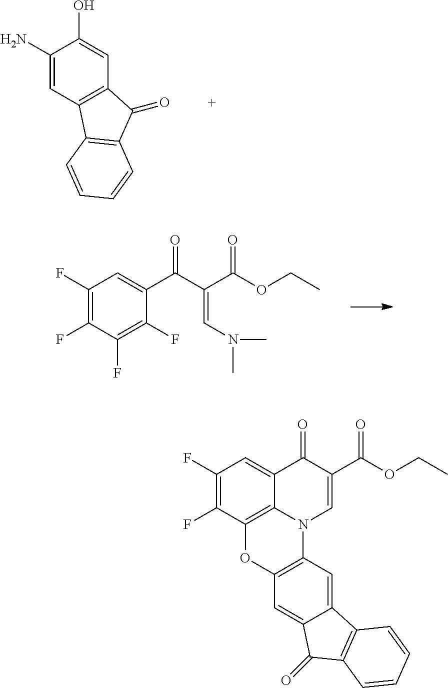

A mixture of 3-amino-2-hydroxyfluorenone (0.9 g, 4.26 mmol) and ethyl-2-(2′,3′,4′,5′-tetrafluorobenzoyl-)-3-(dimethylamino)-prop-2-enoate (1.36 g, 4.26 mmol) were heated in DMSO (50 ml) under vacuum for 18 hr. The product was extracted using EtOAc/Brine and the organic layers combined and dried to give the product as a red solid. The solid was dissolved in DMSO (40 ml) containing a large excess of K₂CO₃ and heated at 100° C. for 30 min. After cooling to room temperature, brine (30 ml) was added and the precipitated product collected as a yellow solid (60% over two steps) (M+1, 446).

### Example 41

### Preparation of 1,2-Difluoro-4-oxo-4H-pyrido[3,2,1-kl]-8-fluorenone-5-carboxylic acid

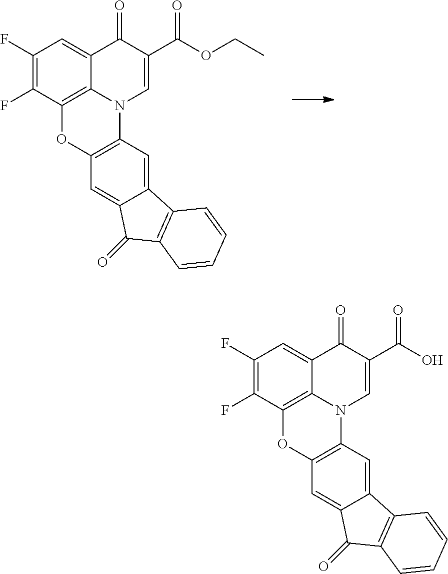

A mixture of ethyl 1,2-difluoro-4-oxo-4H-pyrido[3,2,1-kl]-8-fluorenone-5-carboxylate in acetic acid:conc. HCl (1:1) (50 ml each) was heated at reflux for 2 hr. After cooling to room temperature, water (50 ml) was added and the product collected as yellow solid (94%) (M+1, 418).

### Example 42

### Preparation of Ethyl 1,2-Difluoro-4-oxo-4H-pyrido[3,2,1-kl]-8-anthraquinone-5-carboxylate

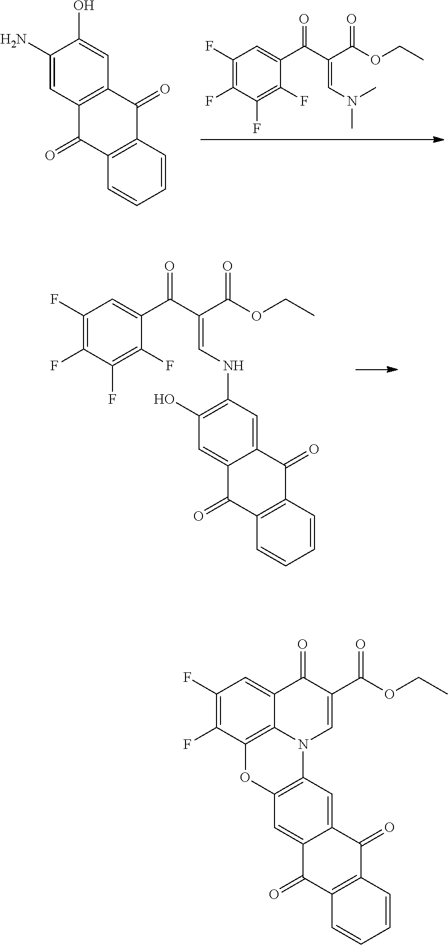

A mixture of 3-amino-2-hydroxyanthraquinone (5.54 g, 23.2 mmol) and ethyl-2-(2′,3′,4′,5′-tetrafluorobenzoyl-)-3-(dimethylamino)-prop-2-enoate (8.7 g, 34.8 mmol) were heated in a minimum of DMSO (˜10 ml) under vacuum for 24 hr. The product was precipitated by the addition of water (50 ml). The solid was dried overnight in a vacuum oven and dissolved in DMSO (40 ml) containing a large excess of K₂CO₃ and heated at 100° C. for 30 min. After cooling to room temperature, brine (30 ml) was added and the precipitated product collected as a yellow solid (60% over two steps) (M+1, 474).

### Example 43

### Preparation of 1,2-Difluoro-4-oxo-4H-pyrido[3,2,1-kl]-8-anthraquinone-5-carboxylic acid

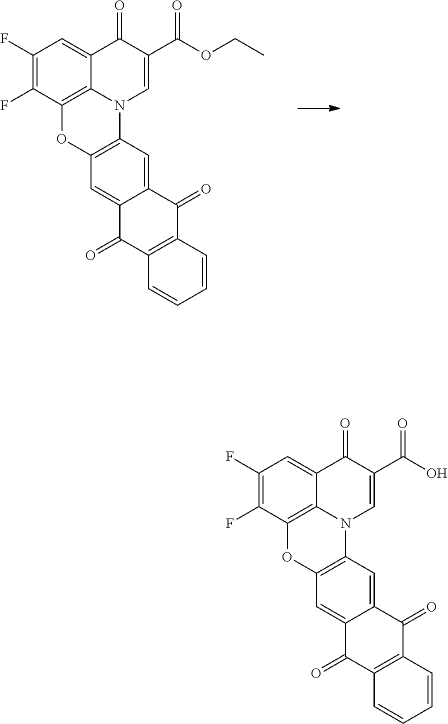

A mixture of ethyl 1,2-difluoro-4-oxo-4H-pyrido[3,2,1-kl]-8-anthraquinonenone-5-carboxylate (3.5 g, 6.8 mmol) in acetic acid:conc. HCl (1:1) (50 ml each) was heated at reflux for 2 hr. After cooling to room temperature, water (50 ml) was added and the product collected as yellow solid (94%) (M+1, 446).

### Example 44

### Preparation of Ethyl 1,2-Difluoro-4-oxo-4H-pyrido[3,2,1-kl]-8-phenyl-phenoxazole-5-carboxylate

### 2-amino(t-butoxy carbonyl)-5-amino hydroquinone

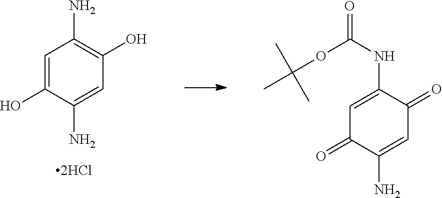

A solution of Boc anhydride (7.17 g, 33 mmol) and DIEA (17 ml, 99 mmol) in DMSO (20 ml) was added dropwise at room temperature to stirred solution of 1,4-dihydroxy-2,5-diaminobenzene (7 g, 33 mmol). After stirring for 18 hr, the product was separated between EtOAc and brine and the organic layers combined and dried over MgSO₄. After removal of solvent the residue was subjected to column chromatography on silica eluting with 25-50% EtOAc in hexane to give pure product (45%) (M+1, 239).

### 4-hydroxy-3-amino(t-butoxy carbonyl)-phenoxazole

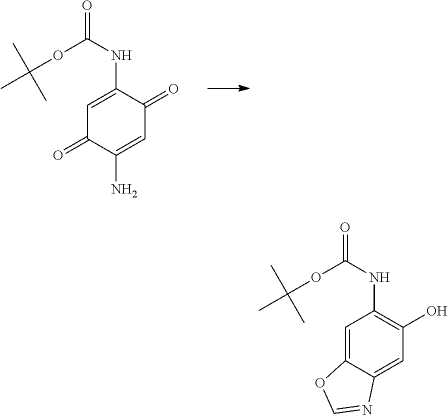

To solution of 2-amino(t-butoxy carbonyl)-5-amino hydroquinone (4.69 g, 23.3 mmol) dissolved in acetonitrile/water (1:1; 20 ml) was added Na hydrosulfite (large excess) and the mixture stirred at room temp. for 15 min The acetonitrile was removed in vacuo and the aqueous mixture extracted with EtOAc (3×20 ml). Combined organic layers were dried over MgSO₄ and solvent removed in vacuo. The residue was taken up in neat triethyl orthoformate (100 ml), left to stir for 16 hr then heated to reflux for 10 min. The product was precipitated following cooling to room temp. by the addition of water (83%) (M+1, 251).

### 4-hydroxy-2-amino phenoxazole

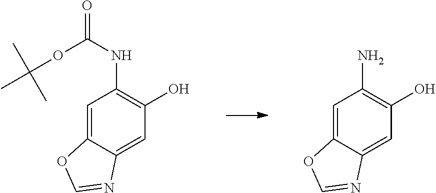

4-Hydroxy-3-amino(t-butoxy carbonyl)-phenoxazole (3 g, 12 mmol) was dissolved in neat TFA (100 ml) and allowed to stir at room temperature for 1 hour. TFA was removed in vacuo to leave the final product as a TFA salt (quant.) (M−1, 149)

### Ethyl-2-(2′,3′,4′,5′-tetrafluorobenzoyl-)-3-(N-(4-hydroxy-2-amino phenoxazole))-prop-2-enoate

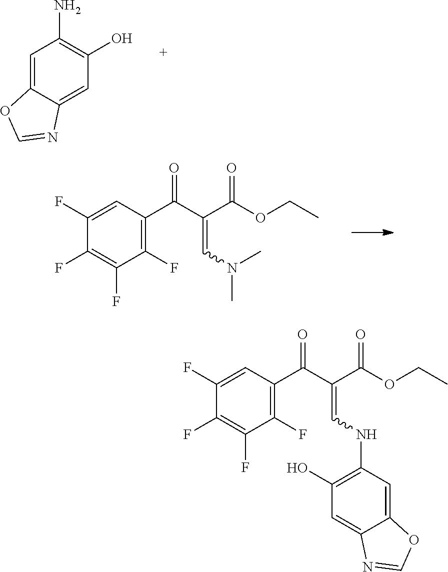

A solution of ethyl-2-(2′,3′,4′,5′-tetrafluorobenzoyl-)-3-(dimethylamino)-prop-2-enoate (7.34 g, 23 mmol) and 2-amino-4-phenyl-phenol (3.45 g, 23 mmol) in EtOAc (20 ml) containing triethylamine (10 ml) was stirred under vacuum on the rotary evaporator for 3 hours. The EtOAc was removed in vacuo and the residue subjected to column chromatography on silica eluting with 50% EtOAc in hexane to give pure product (72%) (M+1, 425).

### Ethyl 1,2-Difluoro-4-oxo-4H-pyrido[3,2,1-kl]-8-phenyl-phenoxazole-5-carboxylate

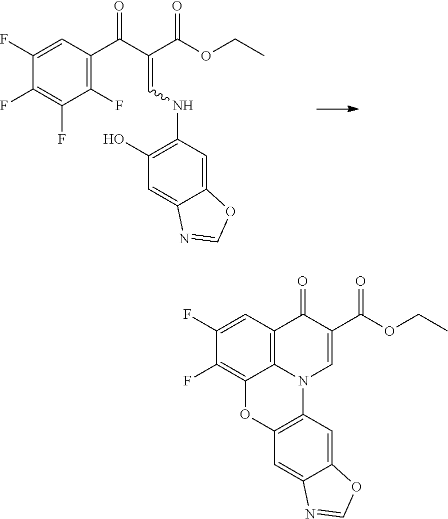

A solution of ethyl-2-(2′,3′,4′,5′-tetrafluorobenzoyl-)-3-(N-(4-hydroxy-2-amino phenoxazole))-prop-2-enoate (3.5 g, 8.25 mmol) in DMSO (50 m) containing K₂CO₃ (large excess) was heated at 80° C. for 10 min. After cooling to room temperature, water was added to precipitate the product as a yellow sold (82%) (M+1, 385).

### Example 45

### Preparation of 1,2-Difluoro-4-oxo-4H-pyrido[3,2,1-kl]-8-(3′-hydroxy-4′-amino phenyl)-5-carboxylic acid

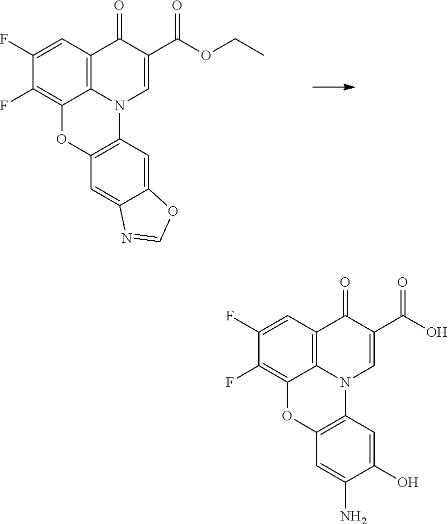

A mixture of ethyl 1,2-difluoro-4-oxo-4H-pyrido[3,2,1-kl]-8-phenyl-phenoxazole-5-carboxylate (2.3 g, 6 mmol) in acetic acid:conc. HCl (1:1; 100 ml) was heated to reflux for 30 min. After cooling to room temp., volatiles were removed in vacuo to leave the product as a brown solid (82%) (M+1, 347).

### Example 46

### Preparation of 1,2-Difluoro-4-oxo-4H-pyrido[3,2,1-kl]-8-(nitro-phenoxazine)-5-carboxylic acid

### 1,2-Difluoro-4-oxo-4H-pyrido[3,2,1-kl]-8-(3′-hydroxy-4′-amino-(N-2″-fluoro-4″-nitro phenyl)-phenyl))-5-carboxylic acid

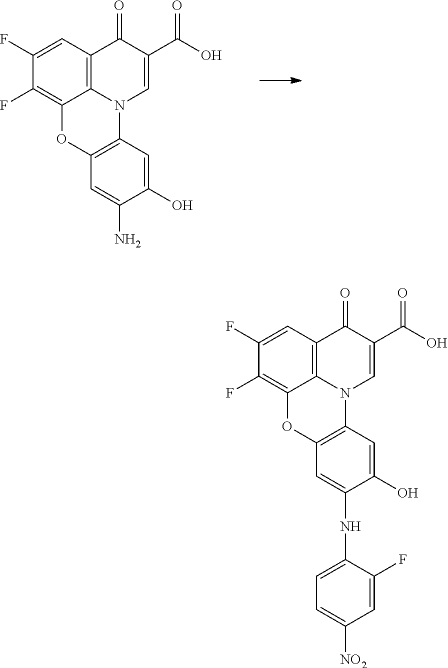

A solution of 1,2-difluoro-4-oxo-4H-pyrido[3,2,1-kl]-8-(3′-hydroxy-4′-amino phenyl)-5-carboxylic acid (0.5 g, 1.44 mmol), 3,4-difluoro-nitro benzene (0.5 ml, 4.3 mmol) and DIEA (1 ml) was heated to 90° C. in NMP (50 ml) for 30 min After cooling to room temp. the product was precipitated by the addition of water and filtered (63%) (M+1, 486).

### 1,2-Difluoro-4-oxo-4H-pyrido[3,2,1-kl]-8-(nitro-phenoxazine)-5-carboxylic acid

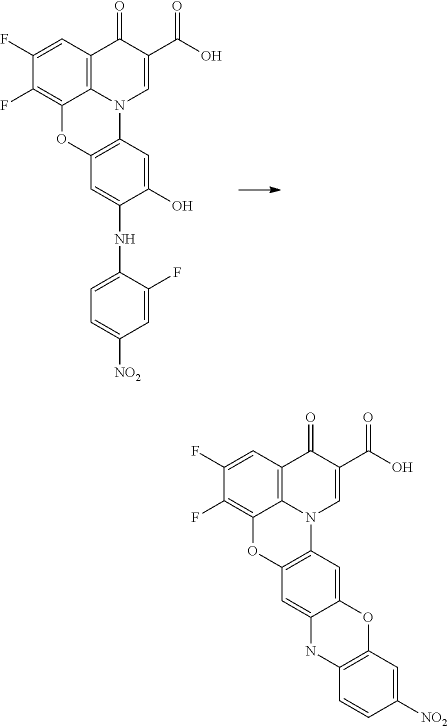

A solution of 2-difluoro-4-oxo-4H-pyrido[3,2,1-kl]-8-(3′-hydroxy-4′-amino phenyl)-5-carboxylic acid (0.3 g, 0.6 mmol) in DMSO (50 ml) containing an excess of K₂CO₃ was stirred and heated to 110° C. for 1 hr. After cooling to room temp. the product was precipitated by the addition of 3M HCl and filtered (71%) (M+1, 465).

### Example 47

### Preparation of 1,2-Difluoro-4-oxo-4H-pyrido[3,2,1-kl]-8-(amino-phenoxazine)-5-carboxylic acid

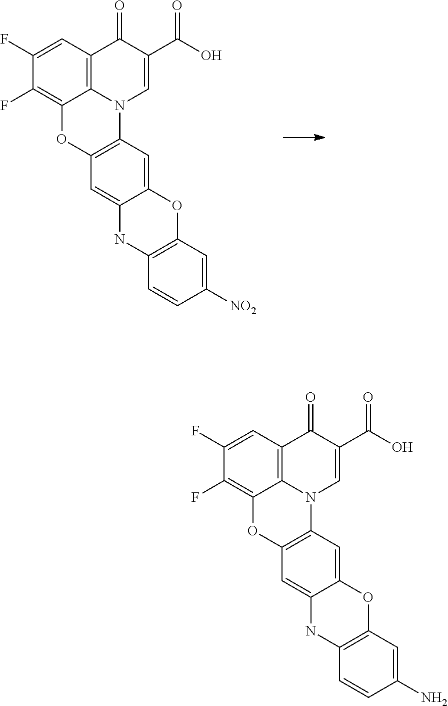

A mixture of 1,2-difluoro-4-oxo-4H-pyrido[3,2,1-kl]-8-(nitro-phenoxazine)-5-carboxylic acid (0.1 g, 0.2 mmol) and Tin (II) chloride (0.15 g, 0.6 mmol) in acetic acid:conc. HCl (1:1; 50 ml) was heated to reflux for four hr. After cooling to room temp. the product was precipitated by the addition of water and filtered (72%) (M+1, 435).

### Example 48

### Preparation of Hydrazide Analogs from Corresponding Esters

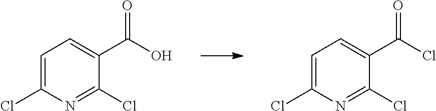

2,6-dichloronicotinic acid (2.70 g, 11 mmol) was suspended in dichloromethane (10 mL) and treated with oxalyl chloride (1.74 g, 14 mmol). The mixture was cooled in an ice bath and 2 drops of dimethylformamide was added. After an initial vigorous outgassing, the ice bath was removed and the solution was stirred for 18 hours at room temperature. An aliquot was quenched with methanol and analyzed by LCMS indicating that all the acid had been converted to the acid chloride. The solution was concentrated on a rotary evaporator to give the acid chloride as a light brown crystalline solid which was used in the subsequent step without further purification. LCMS: 206.2 (methyl ester M+H).

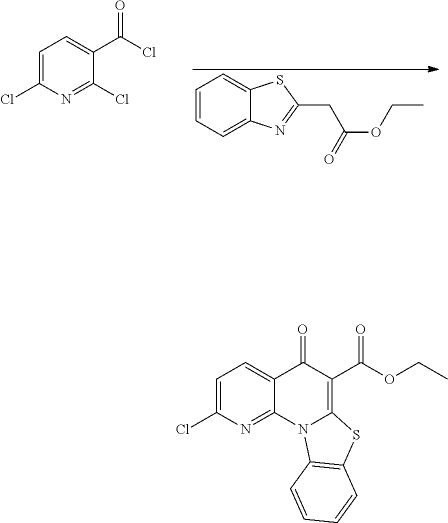

Tetrahydrofuran (25 mL) was added to a mixture of ethyl 2-(benzothiazol-2-yl)acetate, magnesium chloride (2.21 g, 10 mmol) and 2,6-dichloronicotinyl chloride (11 mmol). The resulting suspension was cooled in an ice bath and triethylamine (2.02 g, 20 mmol) was added dropwise at such a rate that the internal temperature did not go over 10° C. as measured with an internal thermocouple probe. Once the addition was complete, the ice bath was removed and the mixture was stirred at room temperature for 5 hours. The desired chloroester was isolated by diluting the reaction mixture with water, extraction with dichloromethane (2×150 ml) and drying the resulting organic phase with sodium sulfate. Purification by trituration with diethyl ether yielded 2.71 g (76% based on ethyI2-(benzothiazol-2-yl)acetate) as fluffy beige crystals. IHNMR (CDC13, 400 MHz) 9.55 (1H, d, 8.4 Hz), 8.86 (1H, d, 8.4 Hz), 7.77 (1H, dd, 7.6, 1.2 Hz), 7.61 (1H, m), 7.56 (1H, d, 8.4 Hz), 7.49 (1H, m), 4.53 (2H, q, 7.2 Hz), 1.50 (3H, t, 7.2 Hz) 13CNMR (CDC13, 100 MHz) 171.1, 167.4, 163.1, 152.9, 148.4, 140.5, 137.7, 128.5, 127.8, 126.6, 123.1, 122.1, 121.7, 120.5, 106.3, 62.0, 14.7 LCMS: 359.3 (M+H).

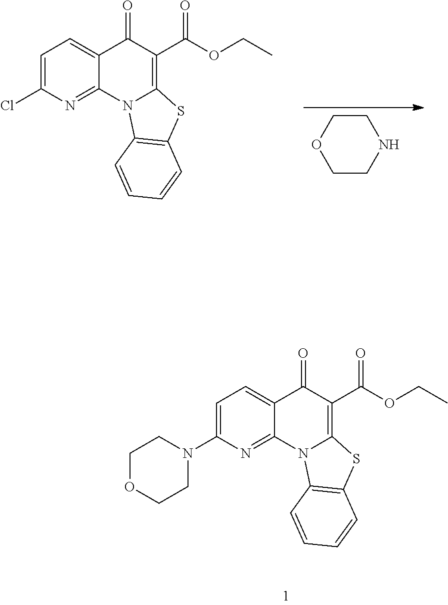

To a slurry of the chloroester (15.00 g, 41.81 mmol) in ACN (150 mL) was added morpholine (7.5 mL, 85.74 mmol) and the mixture was heated at reflux over night. The reaction was cooled to rt and the ppt was collected by filtration. The solid was then dissolved in CHCl₃ (600 mL) and passed through a pad of celite. The solvent was removed under reduced pressure and the resulting solid was triturated in ACN to give the desired product as an off-white solid (14.00 g, 82%). ¹H NMR (CDCl₃) δ: 9.36 (d, 1H), 8.60 (d, 1H), 7.70 (dd, 1H), 7.47 (m, 1H), 7.37 (m, 1H), 6.84 (d, 1H), 4.51 (q, 2H), 3.92 (t, 4H), 3.77 (t, 4H), 1.49 (t, 3H). LCMS (ES): m/z 410 [M+1]⁺.

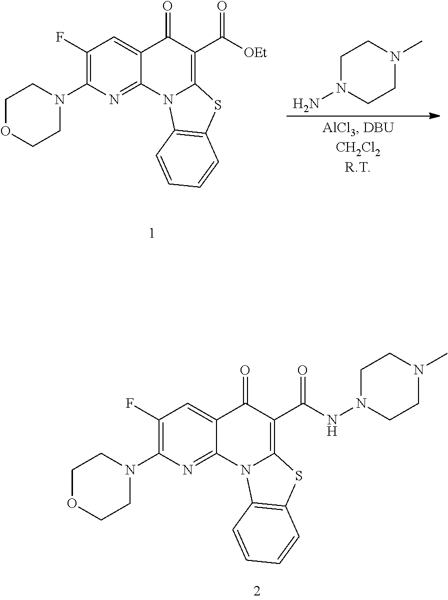

To compound 1 (131 mg, 0.306 mmole) in dichloromethane (10 mL) was added 1-methyl-4-aminopiperazine (0.13 mL, 1.649 mmole), aluminum chloride (112 mg, 0.839 mmole) and DBU (0.13 mL). The mixture was stirred for 1 hr. at room temperature. The mixture was diluted with 100 mL of dichloromethane and 32 mL of sodium potassium tartrate was added followed by 32 mL of 6N solution of sodium hydroxide. The mixture was stirred for 10 min and the organic layer was separated and washed with water (2×20 mL), brine (50 mL), dried over sodium sulfate and concentrated under vacuum to give compound 2 as white solid. The compounds in the Table herein were similarly prepared from corresponding esters.

Alternatively, the hydrazides and hydroxamates of the invention can be prepared from free carboxylic acids that are known in the art, using conventional coupling conditions.

### Example 49

### Antitumor Data

Two xenograft models for inoculation are harvested and diluted to a concentration of 50×10⁶ cells/ml or 100×10⁶ cells/ml. Four to six week old nude mice were injected with 0.1 ml of the cell suspension which contains between 5×10⁶ and 10×10⁶ cells. When tumors are of a suitable size compound dosing is commenced. Tumor sizes are measured throughout the treatment period with calipers and body weights also measured.

### Example 50

### Cell Proliferation and/or Cytotoxicity Assay

Cell Culture

Human cervical epithelial cells (HeLa cells) were obtained from American Type Culture Collection (Manassas, Va.). Cells were grown in Eagle's minimum essential medium (MEM, Hyclone, Utah) supplemented with 2 mM Glutamine, 0.1 mM nonessential amino acid, 1 mM Na Pyruvate, 1.5 g/L NaHCO₃, 50 mg/L gentamicin, and 10% fetal bovine serum (Hyclone, USA) in a humidified atmosphere of 5% CO₂ at 37° C.

MTS Assay

Antiproliferative effects of anticancer drugs may be tested by the CellTiter 96 AQᵤₑₒᵤₛ assay (Promega, WI), which is a colorimetric assay for determining the number of viable cells. (See, e.g., Wang, L., et al., Methods Cell Sci (1996) 18:249-255).

Cells (4,500 cells/well) were seeded on 96 well flat bottom plates (Corning, N.Y.) in 100 μl of culture medium without any anticancer drug on day 0, and the culture medium exchanged for that containing anticancer drug candidates at various concentrations on day 1. After incubation for 3 days under normal growth conditions (on day 4), the monolayers were washed once in PBS, and the medium was switched to 100 n1 of PBS in each of the 96 well plate. After mixing MTS and PMS at the ratio of 20:1, 20 n1 of MTS/PMS solution was added to each of the 96 well plate and incubated for 4 hours in a humidified atmosphere of 5% CO₂ at 37° C. The absorbance was read at 490 nm using FLUOstar Galaxy 96 well plate reader (BMG Labtechnologies, Germany). Micromolar concentrations (MTS data) are concentrations at which 50% of antiproliferative cell response is seen.

### Example 51

### Measurement of mRNA Levels in Cells

Real-time quantitative PCR (QPCR) method was used to detect accurately the changes of the target c-myc and the endogenous reference GAPDH gene copies in the same tube. Cells (15,000 cells/well) were seeded on 96 well flat bottom plates (Corning, N.Y.) and incubated under normal growth conditions for overnight. The next day, the culture medium was exchanged for that contained anticancer drugs at various concentrations and incubate for 4 hrs in a humidified atmosphere of 5% CO₂ at 37° C. Total RNA (tRNA) was extracted using the RNeasy 96 Kit (QIAGEN, CA). The concentration of the tRNA was determined by the RiboGreen RNA Quantitation Reagent (Molecular Probes, OR).

Reverse-transcription (RT) reaction was occurred using 50 ng of tRNA from each well in a 25 n1 reaction containing 1×TaqMan RT buffer, 2.5 uM random hexamers, 5.5 mM MgCl₂, 0.5 mM each deoxynucleoside triphosphate (dNTP), 30 U MultiScribe Reverse Transcriptase, and 10 U RNase inhibitor. RT reactions were incubated for 10 mM at 25° C., reverse-transcribed for 30 min at 48° C., inactivated for 5 min at 95° C., and placed at 4° C. All RT reagents were purchased from Applied Biosystems, CA.

Real-Time QPCR reaction was performed in a 50 μl reaction containing the 5 μl of cDNA, 1× Universal PCR Master Mix, 1×c-myc Pre-Developed Primers and Probe set, and 0.8×GAPDH Pre-Developed Primers and Probe set. Because of the relative abundance of GAPDH gene in Hela, GAPDH primers and probe concentration were adjusted to get accurate threshold cycles (C_{T}) for both genes in the same tube. The threshold cycle (C_{T}) indicates the fractional cycle number at which the amount of amplified target reaches a fixed threshold. By doing so, the GAPDH amplification was stopped before it can limit the common reactants available for amplification of the c-myc, resulted in a reduction in ΔRn value of GAPDH, but no effect on its C_{T} value, and equal amplification efficiency for both genes. The ΔRn value represents the normalized reporter signal minus the baseline signal. ΔRn increases during PCR as amplicon copy number increases until the reaction approaches a plateau.

The c-myc probe was labeled with 6FAM™ dye-MGB and the GAPDH probe was labeled with VIC™ dye-MGB. Preincubation was performed for 2 min at 50° C. to activate AmpErase UNG enzyme and then for 10 min at 95° C. to activate AmpliTaq DNA Polymerase. DNA was amplified for 40 cycles of 15 sec at 95° C. and 1 min at 60° C. Human c-myc and GAPDH cDNA were amplified, detected, and quantitated in real time using the ABI Prism 7000 Sequence Detection system (Applied Biosystems, CA), which was set to detect both 6-FAM and VIC reporter dyes simultaneously.

The data was analyzed by using the ABI PRISM Sequence Detection System and

Microsoft Excel. Relative quantitation was done using the standard curve and comparative C_{T} method at the same time, and both methods gave equivalent results. The cycle at which the amplification plot crosses the C_{T} is known to accurately reflect relative mRNA values. (See, Heid, et al., Genome Res. (1996) 6:986-994; Gibson, et al., Genome Res. (1996) 6:995-1001). QPCR reactions were set up in triplicate at each cDNA sample and the triplicate C_{T} values were averaged. All reagents including Pre-Developed Primers and probe set were purchased from Applied Biosystems, CA. Micromolar concentrations (STOP data) reported are concentrations at which 50% inhibition of c-myc mRNA levels are seen.

### Example 52

### Cell Proliferation and rRNA Modulatory Activities

A representative cell-proliferation assay protocol using Alamar Blue dye (stored at 4° C., use 20 ul per well) is described hereafter.

### 96-Well Plate Setup and Compound Treatment

a. Split and trypsinize cells.

b. Count cells using hemocytometer.

c. Plate 4,000-5,000 cells per well in 100 μl of medium and seed into a 96-well plate according to the following plate layout. Add cell culture medium only to wells B10 to B12. Wells B1 to B9 have cells but no compound added.

| | | | |
|---|---|---|---|
| 1 2 3 | 4 5 6 | 7 8 9 | 10 11 12 |

| | |
|---|---|
| A | EMPTY |

| B | NO COMPOUND ADDED | | | Medium | |
|---|---|---|---|---|---|
| | | | | Only | |
| C | 10 nM | 100 nM | 1 uM | 10 uM | Control |
| D | 10 nM | 100 nM | 1 uM | 10 uM | Comp1 |
| E | 10 nM | 100 nM | 1 uM | 10 uM | Comp2 |
| F | 10 nM | 100 nM | 1 uM | 10 uM | Comp3 |
| G | 10 nM | 100 nM | 1 uM | 10 uM | Comp4 |

| | |
|---|---|
| H | EMPTY |

d. Add 100 μl of 2× drug dilution to each well in a concentration shown in the plate layout above. At the same time, add 100 μl of media into the control wells (wells B10 to B12). Total volume is 200 μl /well.

e. Incubate four (4) days at 37° C., 5% CO2 in a humidified incubator.

f. Add 20 μl Alamar Blue reagent to each well.

g. Incubate for four (4) hours at 37° C., 5% CO2 in a humidified incubator.

h. Record fluorescence at an excitation wavelength of 544 nm and emission wavelength of 590 nm using a microplate reader.

In the assays, cells are cultured with a test compound for approximately four days, the dye then is added to the cells and fluorescence of non-reduced dye is detected after approximately four hours. Different types of cells can be utilized in the assays (e.g., HCT-116 human colorectal carcinoma cells, PC-3 human prostatic cancer cells and MiaPaca human pancreatic carcinoma cells). Anti-proliferative effects of representative compounds are provided in the Table below (i.e., “AB: HCT-116” and “AB: MiaPaCa” columns).

A representative assay for determining whether a compound modulates rRNA production is set forth hereafter. Assays were conducted to determine the effects of compounds on rRNA synthesis from 45S rDNA. Synthesized rRNA was quantified by a polymerase chain reaction (PCR) assay. A primer/probe set was designed using Primer Express software and synthesized by Applied Biosystems. The 5′ ETS Probe utilized had the following sequence (at its 3′ end): 6FAM-TTG ATC CTG CCA GTA GC-MGBNFQ. The primer sequences were as follows:

Forward Primer: CCG CGC TCT ACC TTA CCT ACC T
Reverse Primer: GCA TGG CTT AAT CTT TGA GAC AAG.

A control assay that detected effects of the compounds on C-myc transcription also was conducted using a primer/probe set purchased from ABI (TaqMan Gene Expression Assay with assay ID: Hs99999003_m1). The following assay protocol was utilized:

Step 1. Reverse transcription of RNA to DNA

Mix

1 ug RNA

2.5 ul 10×Taq Man buffer

5.5 ul 25 mM MgCl₂

5 ul of a mix of dNTP (500 uM each)

1.2 ul random hexamer primer (2.5 uM stock)

0.5 ul RNase inhibitor (0.4 units/ul)

0.6 ul Reverse Transcriptase (1.2 units/ul)

bring to 25 ul total volume with water

Incubate at 48 degrees C. for 30 minutes

Inactivate Reverse Transcriptase by incubating at 95 for 5 minutes

Step 2. PCR

Mix

5 ul Reverse Transcriptase reaction product

12.5 ul 2×PCR mix

1 uM forward primer

1 uM reverse primer

0.5 uM Taq Man probe

500 nM Rox

Adjust to 25 ul final volume with water

PCR cycles

95 degrees C. 10 minute

40 cycles of

95 degrees C. 15 seconds

60 degrees C. 1 minute.

Fluorescence of digested label was detected and quantified. The effect of the compounds on rRNA synthesis were assessed in the assay, and provided in the table hereafter are IC50 values of selected compounds pertaining to rRNA synthesis and cMYC RNA synthesis.

Using the above methods, the compounds in Table 1 were prepared and tested, and effects on cell proliferation and rRNA synthesis were assessed.

TABLE 1

| | | | QPCR: | QPCR: | AB: | |
|---|---|---|---|---|---|---|
| Cmpd | | AB: HCT- | rDNA | cMYC | MiaPaCa | MS |
| No. | Structure | 116 (uM) | (uM) | (uM) | (uM) | (M + 1) |
| 1 | 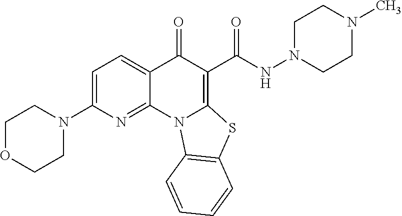 | 0.096 | 0.148 | 1.194 | | 479 |
| 2 | 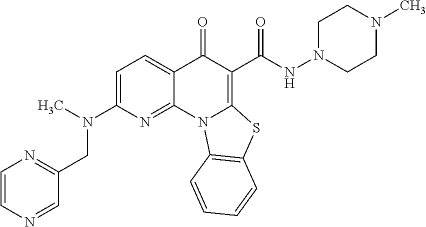 | 0.249 | 0.696 | | 1.479 | 515 |
| 3 | 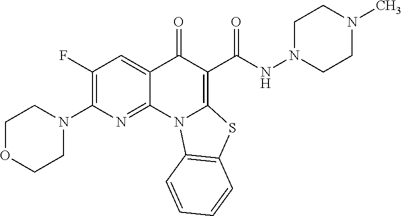 | 0.265 | 0.102 | 6.062 | 0.225 | 497 |
| 4 | 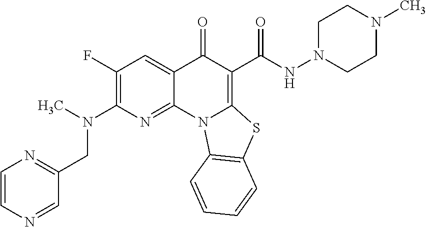 | 0.517 | 0.291 | 7.012 | 0.173 | 533 |
| 5 | 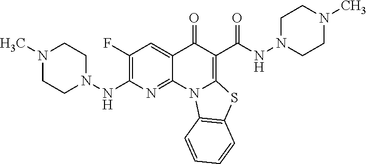 | 1.751 | 0.632 | | 0.486 | 525 |
| 6 | 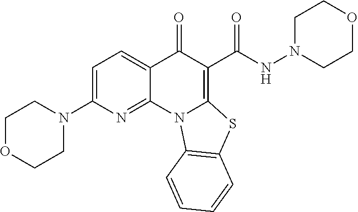 | 0.562 | 0.702 | | 0.549 | 466 |
| 7 | 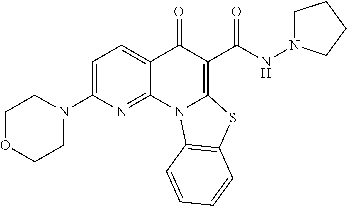 | 0.653 | 0.714 | | 0.955 | 450 |
| 8 | 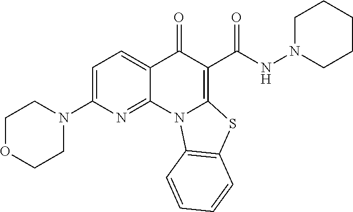 | 50 | 0.458 | 23.613 | 50 | 464 |
| 9 | 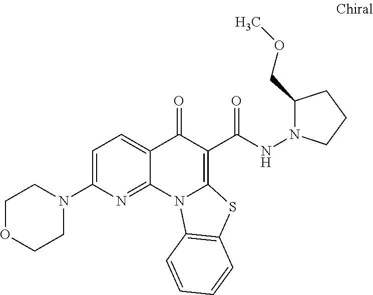 | 1.169 | 0.72 | | 0.596 | 494 |
| 10 | 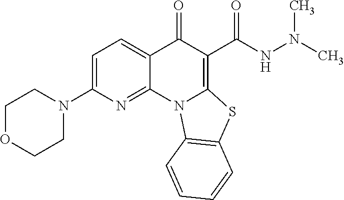 | 0.523 | 0.414 | 8.99 | 0.444 | 424 |
| 11 | 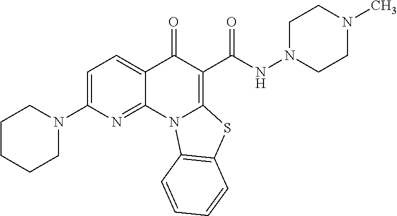 | 0.915 | 3.142 | | 0.74 | 477 |
| 12 | 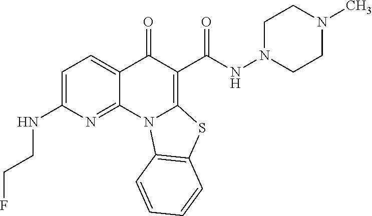 | 0.693 | 1.577 | | 1.034 | 455 |
| 13 | 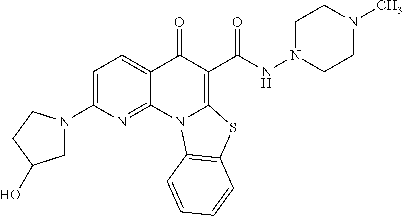 | 5.877 | 7.323 | | 3.312 | 479 |
| 14 | 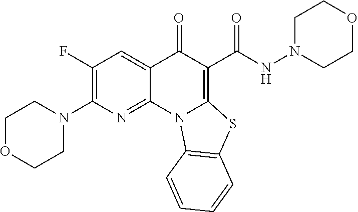 | 0.69 | 2.242 | | 8.091 | 484 |
| 15 | 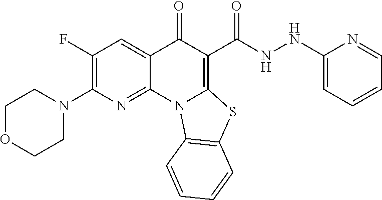 | 1.308 | 5.551 | | 5.36 | 491 |
| 16 | 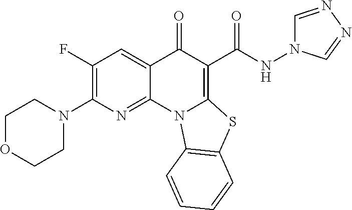 | 0.292 | 0.35 | 14.026 | 0.715 | 466 |
| 17 | 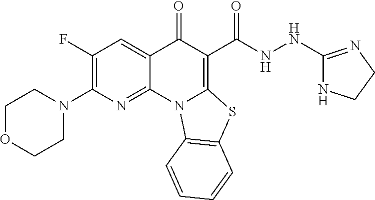 | 0.001 | 0.038 | 1.104 | 0.003 | 482 |
| 18 | 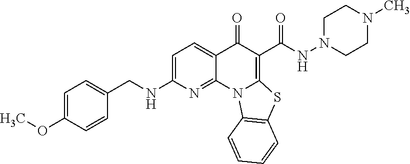 | 0.985 | 1.29 | | 0.893 | 529 |
| 19 | 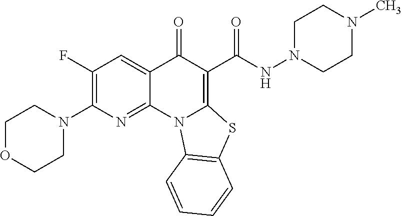 | 0.416 | 0.514 | | 0.946 | 494 |
| 20 | 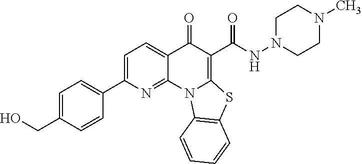 | 7.143 | 9.712 | | 50 | 500 |
| 21 | 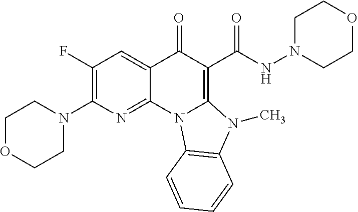 | 0.662 | | | 1.302 | 481 |
| 22 | 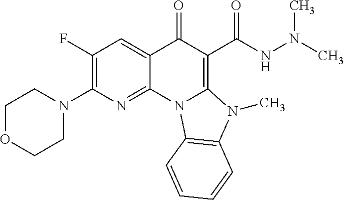 | 1.408 | | | 2.187 | 439 |
| 23 | 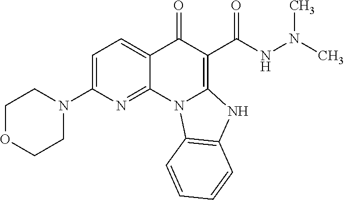 | 1.643 | | | | 407 |
| 24 | 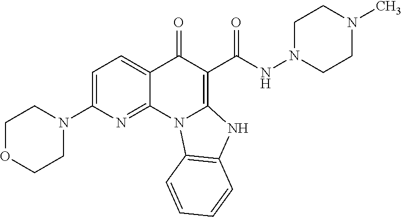 | 1.423 | | | | 462 |
| 25 | 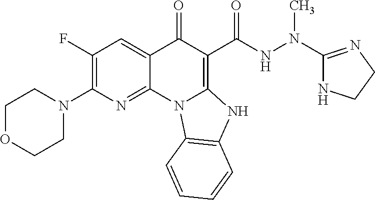 | 0.277 | | | | 496 |
| 26 | | <0.03 | 0.068 | 0.421 | | 493.2 |

It is understood that the foregoing detailed description and accompanying examples are merely illustrative, and are not to be taken as limitations upon the scope of the invention. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. U.S. patents and publications referenced herein are incorporated by reference.

1-28. (canceled)

29. A compound of formula (III): 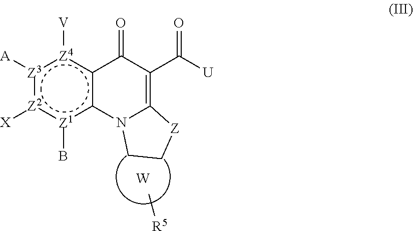
or a pharmaceutically acceptable salt thereof, wherein:
A, V, and X are independently H, halo, azido, R², CH₂R², SR², OR², —NR¹—NR¹R², —NR¹—OR², NR¹R² or an optionally substituted seven-membered carbocyclic or azacyclic ring; or
wherein A and X, or A and V may form a carbocyclic ring, heterocyclic ring, aryl or heteroaryl, each of which may be optionally substituted and/or fused with a cyclic ring; wherein in NR¹R², R¹ and R² together with N may form an optionally substituted ring;
each Z is CH₂, O, S, or NR¹;
Z¹, Z², Z³, and Z⁴ are independently C or N;
U is NR¹NR¹R² or NR¹OR²;
each R¹ is independently H or a C₁₋₆ alkyl;
each R² is independently H or a C₁₋₁₀ alkyl or C₂₋₁₀ alkenyl optionally containing one or more non-adjacent heteroatoms selected from N, O, and S, and optionally substituted with a carbocyclic or heterocyclic ring, which is itself optionally substituted; or R² is an optionally substituted carbocyclic ring, heterocyclic ring, aryl or heteroaryl;
each B is H or halo;
each W is an optionally substituted aryl or heteroaryl, which may be monocyclic, or fused with a single or multiple ring and optionally containing a heteroatom;
and each R⁵ is a substituent at any position on the fused ring; and is H, OR², NR¹R², C₁₋₆ alkyl, C₂₋₆ alkenyl, each optionally substituted by halo, C═O or one or more heteroatoms; or R⁵ is an inorganic substituent; or two adjacent R⁵ is linked to obtain a 5-6 membered substituted or unsubstituted carbocyclic or heterocyclic ring, optionally fused to an additional substituted or unsubstituted carbocyclic or heterocyclic ring.

30. The compound of claim 29, wherein Z is S.

31. The compound of claim 29, wherein Z is NR¹.

32. The compound of claim 29, wherein at least one of Z¹, Z², Z³, and Z⁴ is N.

33. The compound of claim 29, wherein Z¹ is N and Z², Z³, and Z⁴ are each C.

34. The compound of claim 29, wherein A is H or halo and V is H or halo.

35. The compound of claim 29, wherein W is phenyl or naphthyl, each of which is optionally substituted.

36. The compound of claim 29, wherein U is NR¹NR¹R².

37. The compound of claim 29, wherein U is NH—NR¹R², where NR¹R² is an optionally substituted cyclic group.

38. A compound selected from the group consisting of: 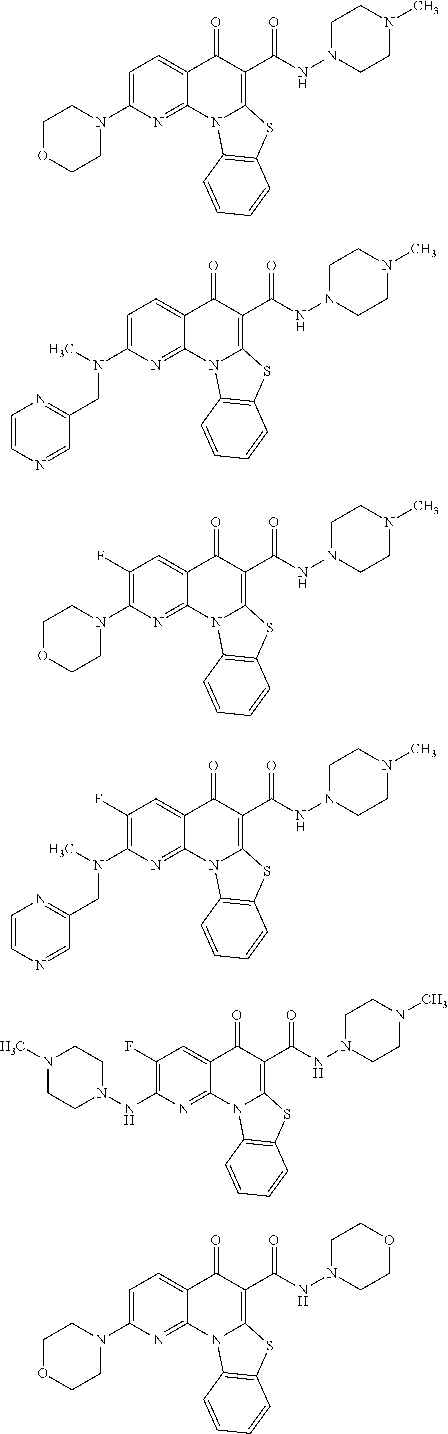 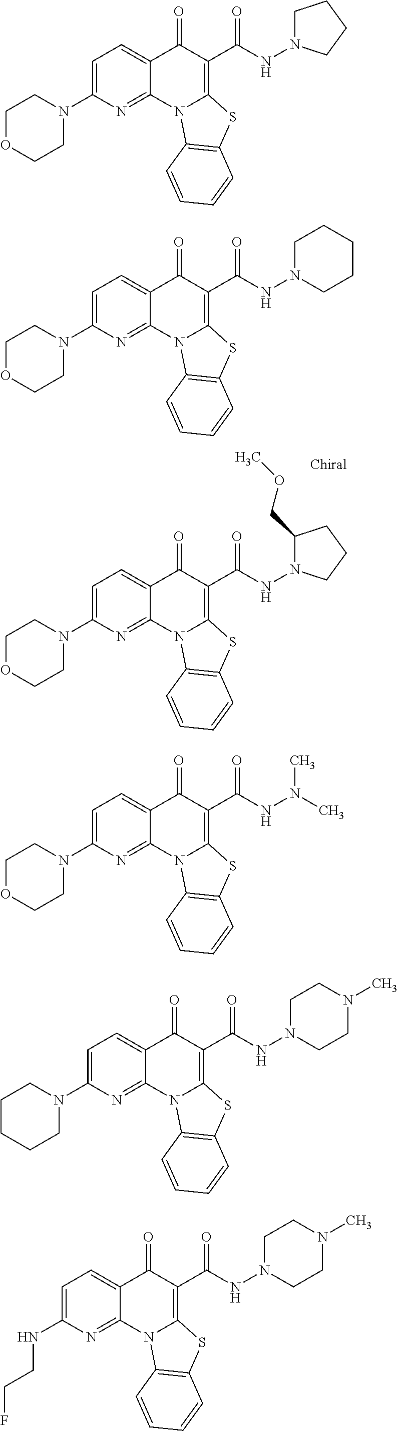 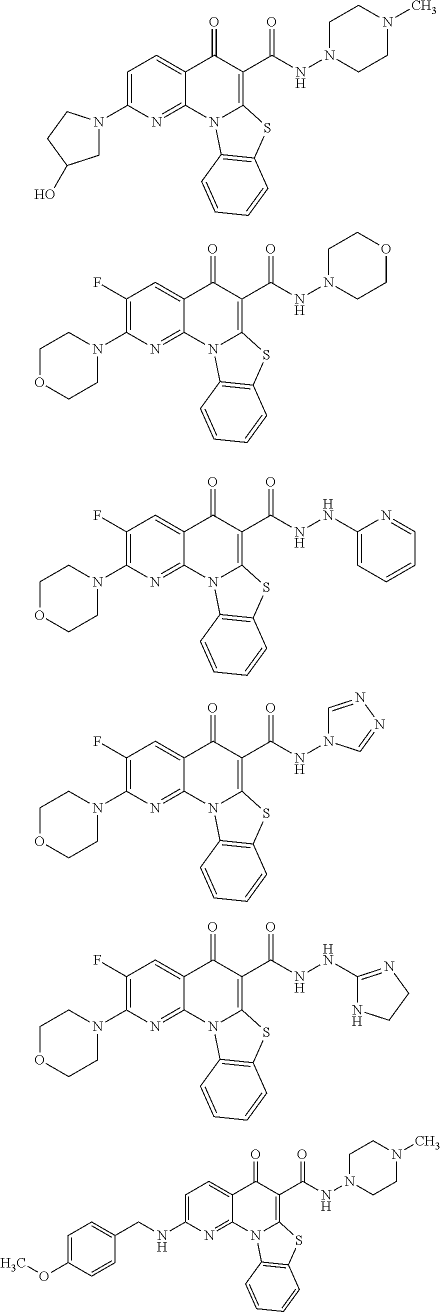 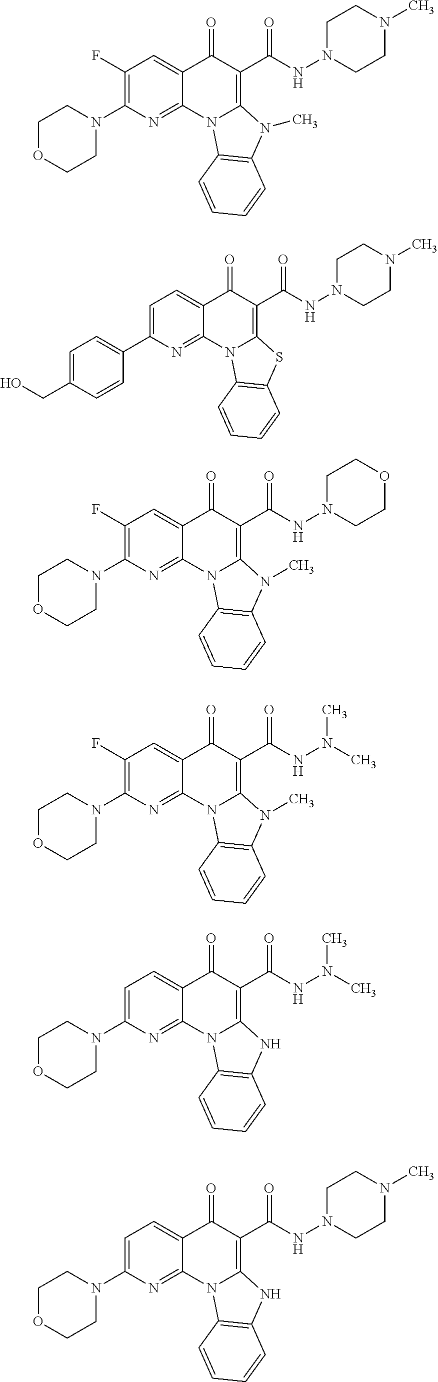 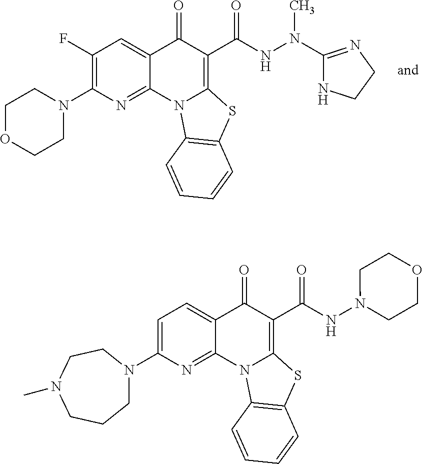
or a pharmaceutically acceptable salt thereof.

39. A pharmaceutical composition comprising a compound of claim 29 or a pharmaceutically acceptable salt thereof, admixed with a pharmaceutically acceptable excipient.

40. A method to treat cancer or an infection caused by a virus, bacterium or fungus, comprising administering to a subject in need of such treatment an effective amount of a compound of Formula (III): 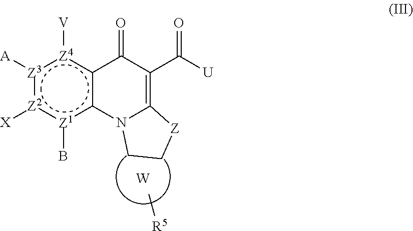
or a pharmaceutically acceptable salt thereof, wherein:
A, V, and X are independently H, halo, azido, R², CH₂R², SR², OR², —NR¹—NR¹R², —NR¹—OR², NR¹R² or an optionally substituted seven-membered carbocyclic or azacyclic ring; or
wherein A and X, or A and V may form a carbocyclic ring, heterocyclic ring, aryl or heteroaryl, each of which may be optionally substituted and/or fused with a cyclic ring; wherein in NR¹R², R¹ and R² together with N may form an optionally substituted ring;
each Z is CH₂, O, S, or NR¹;
Z¹, Z², Z³, and Z⁴ are independently C or N;
U is NR¹NR¹R² or NR¹OR²;
each R¹ is independently H or a C₁₋₆ alkyl;
each R² is independently H or a C₁₋₁₀ alkyl or C₂₋₁₀ alkenyl optionally containing one or more non-adjacent heteroatoms selected from N, O, and S, and optionally substituted with a carbocyclic or heterocyclic ring, which is itself optionally substituted; or R² is an optionally substituted carbocyclic ring, heterocyclic ring, aryl or heteroaryl;
each B is H or halo;
each W is an optionally substituted aryl or heteroaryl, which may be monocyclic, or fused with a single or multiple ring and optionally containing a heteroatom;
and each R⁵ is a substituent at any position on the fused ring; and is H, OR², NR¹R², C₁₋₆ alkyl, C₂₋₆ alkenyl, each optionally substituted by halo, C═O or one or more heteroatoms; or R⁵ is an inorganic substituent; or two adjacent R⁵ is linked to obtain a 5-6 membered substituted or unsubstituted carbocyclic or heterocyclic ring, optionally fused to an additional substituted or unsubstituted carbocyclic or heterocyclic ring.

41. A compound of Formula (I): 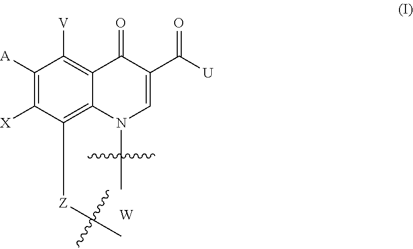
or a pharmaceutically acceptable salt thereof;
wherein V is H, halo, or NR¹R²;
A is H, fluoro, or NR¹₂;
Z is O, S, NR¹ or CH₂;
U is —NR¹NR¹R² or —NR¹OR²;
X is OR², NR¹R², —NR¹—NR¹R², —NR¹—OR², halo, azido, SR² or an optionally substituted seven-membered carbocyclic or azacyclic ring;
n is 1-3;
wherein in NR¹R², R¹ and R² may form a double bond or a ring, each of which is optionally substituted;
each R¹ is independently H or a C₁₋₆ alkyl;
each R² is independently H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring;
or R² is an optionally substituted heterocyclic ring, aryl or heteroaryl;
W is selected from the group consisting of 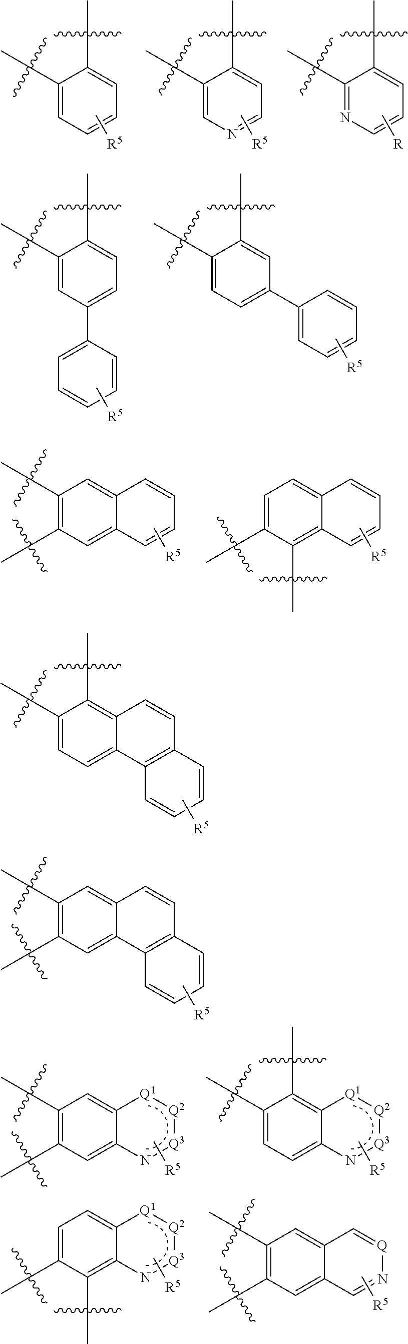 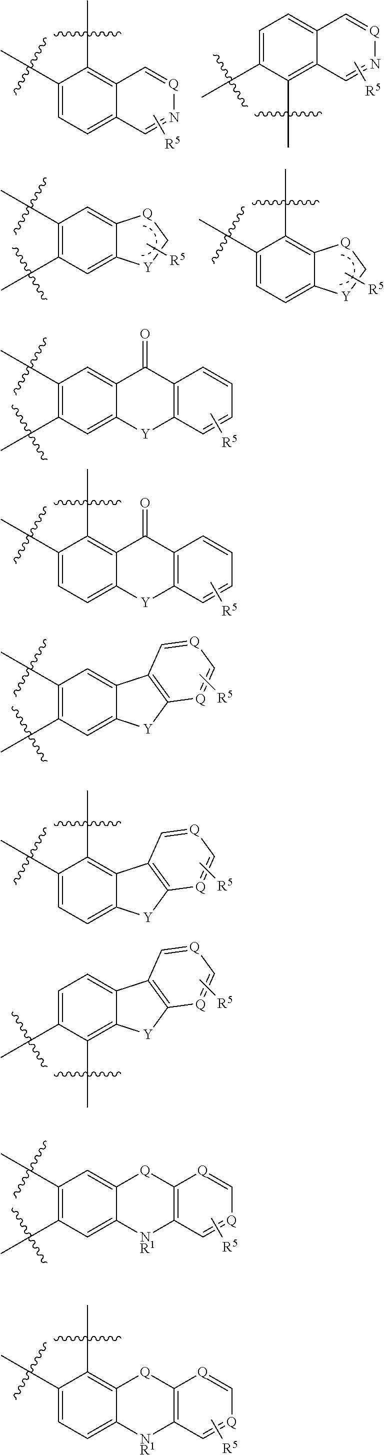 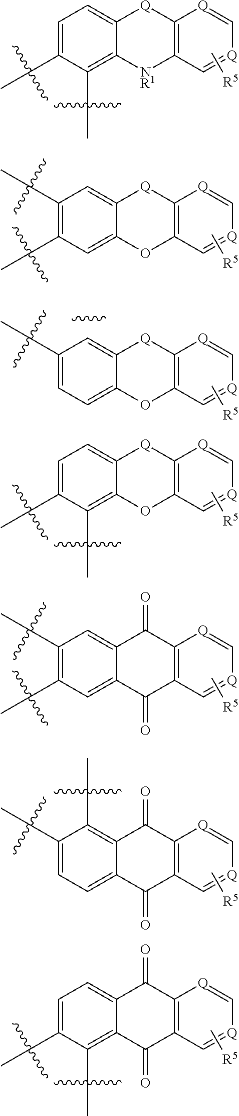
wherein Q, Q¹, Q², and Q³ are independently CH or N;
Y is independently O, CH, ═O or NR¹;
and R⁵ is a substituent at any position on the fused ring; and is H, OR², C₁₋₆ alkyl, C₂₋₆ alkenyl, each optionally substituted by halo, ═O or one or more heteroatoms; or R⁵ is an inorganic substituent; or two adjacent R⁵ groups are linked to obtain a 5-6 membered substituted or unsubstituted carbocyclic or heterocyclic ring, optionally fused to an additional substituted or unsubstituted carbocyclic or heterocyclic ring.

42. The compound of claim 41, wherein A or X is fluoro.

43. The compound of claim 41, wherein V is NH₂ or a group having the formula NR¹—(CR¹₂)ₙ—NR³R⁴;
wherein R¹ and R³ are independently H or C₁₋₆ alkyl;
n is 1-6; and
R⁴ is H, C₁₋₆ alkyl optionally substituted with a carbocyclic or heterocyclic ring, or aryl; and wherein in NR³R⁴, R³ and R⁴ may form an optionally substituted ring.

44. The compound of claim 41, wherein U is NR¹NR¹R² or NR¹—OR², wherein R¹ is H and R² is a C₁₋₁₀ alkyl optionally containing one or more heteroatoms, and optionally substituted with a substituted or unsubstituted ring selected from C₃₋₆ cycloalkyl, aryl and a 5-14 membered heterocyclic ring containing one or more N, O or S.

45. The compound of claim 41, wherein U is NR¹NR¹R² or NR¹—OR², wherein R¹ is H and R² is an aryl or a 5-14 membered heterocyclic ring containing one or more N, O or S, each optionally substituted with an amino or another heterocyclic ring.

46. The compound of claim 41, wherein U is NR¹NR¹R², where R¹ and R² in NR¹R² form an optionally substituted 5-14 membered ring containing one or more N, O or S.

47. The compound of claim 41, wherein U is a group of the formula:
NR¹NR¹—(CR¹₂)ₙ—NR³R⁴ or NR¹O—(CR¹₂)ₙ—NR³R⁴ (2)
wherein R¹ and R³ are independently H or C₁₋₆ alkyl;
n is 1-6; and
R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring;
and wherein in NR³R⁴, R³ and R⁴ may form an optionally substituted ring.

48. The compound of claim 41, wherein X is NR¹R².

49. The compound of claim 41, wherein X is a group of the formula
NR¹—(CR¹₂)ₙ—NR³R⁴ (2)
wherein R¹ and R³ are independently H or C₁₋₆ alkyl;
n is 1-6; and
R⁴ is H or a C₁₋₁₀ alkyl or C₂₋₁₀ alkenyl optionally containing one or more non-adjacent heteroatoms selected from N, O and S, and optionally substituted with a carbocyclic or heterocyclic ring; and
wherein in NR³R⁴, R³ and R⁴ may form an optionally substituted ring.

50. A pharmaceutical composition comprising a compound of claim 41 or a pharmaceutically acceptable salt thereof, and at least one pharmaceutically acceptable excipient.

51. A method to treat cancer or an infection caused by a virus, bacterium or fungus, comprising administering to a subject in need of such treatment an effective amount of a compound of Formula (I): 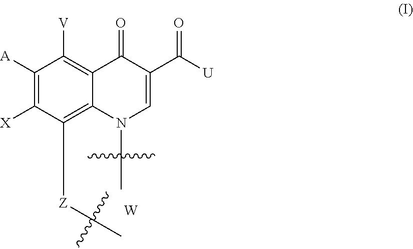
wherein V is H, halo, or NR¹R²;
A is H, fluoro, or NR¹₂;
Z is O, S, NR¹ or CH₂;
U is —NR¹NR¹R² or —NR¹OR²;
X is OR², NR¹R², —NR¹—NR¹R², —NR¹—OR², halo, azido, SR² or an optionally substituted seven-membered carbocyclic or azacyclic ring;
n is 1-3;
wherein in NR¹R², R¹ and R² may form a double bond or a ring, each of which is optionally substituted;
each R¹ is independently H or a C₁₋₆ alkyl;
each R² is independently H or a C₁₋₁₀ alkyl or C₂₋₁₀ alkenyl optionally containing one or more non-adjacent heteroatoms selected from N, O, and S, and optionally substituted with an optionally substituted carbocyclic or heterocyclic ring;
or R² is an optionally substituted heterocyclic ring, aryl or heteroaryl;
W is selected from the group consisting of 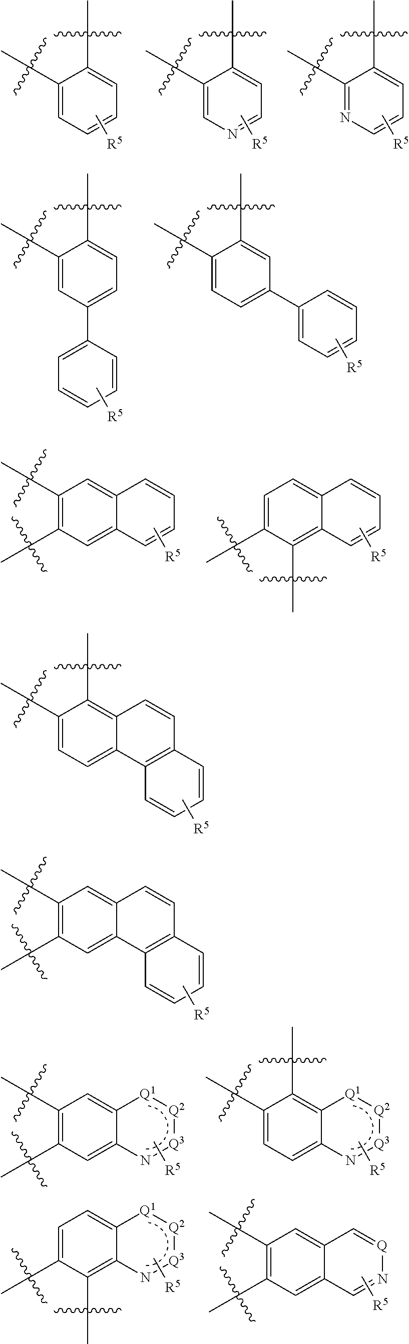 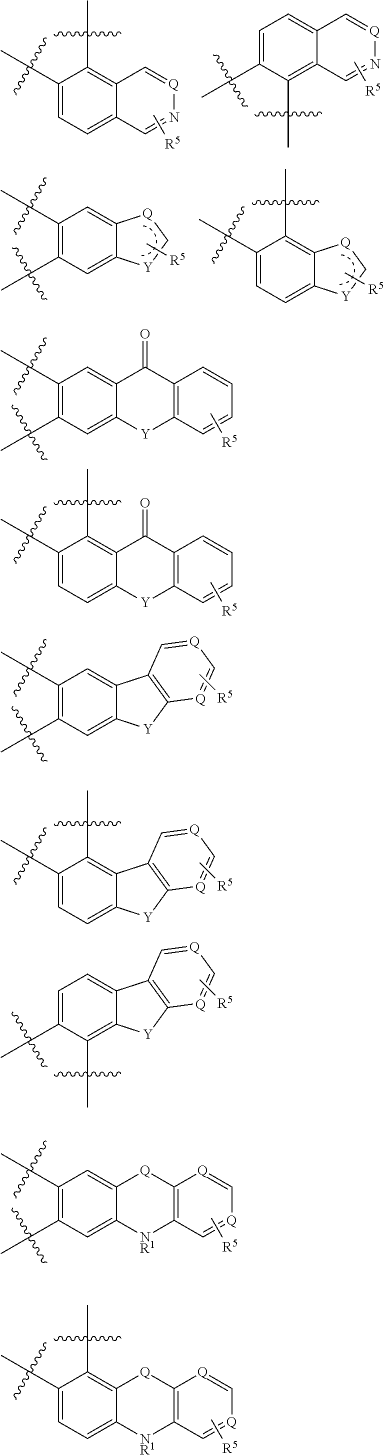 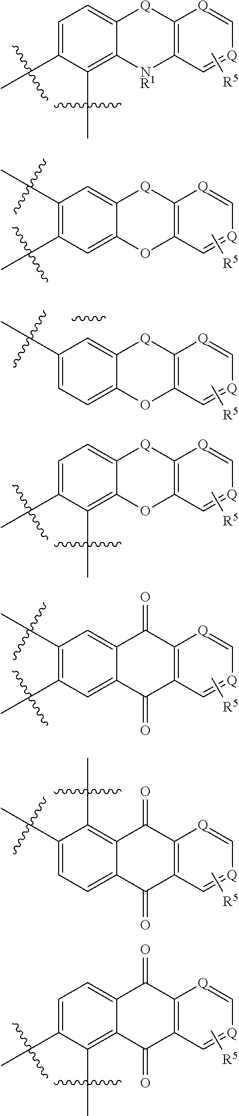
wherein Q, Q¹, Q², and Q³ are independently CH or N;
Y is independently O, CH, ═O or NR¹;
and R⁵ is a substituent at any position on the fused ring; and is H, OR², C₁₋₆ alkyl, C₂₋₆ alkenyl, each optionally substituted by halo, ═O or one or more heteroatoms; or R⁵ is an inorganic substituent; or two adjacent R⁵ groups are linked to obtain a 5-6 membered substituted or unsubstituted carbocyclic or heterocyclic ring, optionally fused to an additional substituted or unsubstituted carbocyclic or heterocyclic ring;
or a pharmaceutically acceptable salt thereof.
